(19) United States
(12) Patent Application Publication
(10) Pub. No.: US 2026/0193242 A1
(43) Pub. Date: Jul. 9, 2026

(54) USEFUL COMPOUNDS FOR MODULATING INFLAMMATION

(71) Applicant: FLAGSHIP PIONEERING INNOVATIONS VI, LLC, Cambridge, MA (US)

(72) Inventors: Alexander Vincent WEST, Cambridge, MA (US); Joseph F. KELLEHER, III, Cambridge, MA (US); Kevin D. KREUTTER, Cambridge, MA (US); Jason PARK, Cambridge, MA (US); Amanda Clare KASPER, Cambridge, MA (US); Nathaniel G. MAHIEU, Cambridge, MA (US); Sabrina Yusang YANG, Cambridge, MA (US)

(21) Appl. No.: 19/128,192

(22) PCT Filed: Nov. 9, 2023
(86) PCT No.: PCT/US2023/079216

§ 371 Date: May 8, 2025

Related U.S. Application Data
(60) Provisional application No. 63/424,061, filed on Nov. 9, 2022.

(51) Int. Cl. C07D 413/04; A61K 31/422; A61K 31/427; A61K 31/4439; A61K 31/454; A61P 1/00; A61P 29/00; C07D 413/14; C07D 417/04; C07D 495/04

(52) U.S. Cl. CPC C07D 413/04; A61K 31/422; A61K 31/427; A61K 31/4439; A61K 31/454; A61P 1/00; A61P 29/00; C07D 413/14; C07D 417/04; C07D 495/04

(57) ABSTRACT

The present disclosure provides compounds of Formula (I), or a pharmaceutically acceptable salt thereof, wherein R to R³ and X are defined herein; pharmaceutical compositions; dosage forms comprising the compounds or pharmaceutical compositions, and methods of treating inflammation, decreasing inflammation, decreasing an inflammatory marker, treating inflammatory bowel disease, such as Crohn's disease or ulcerative colitis, or treating sepsis in a subject in need thereof, comprising administering the compounds, pharmaceutical compositions or dosage forms disclosed herein to a subject in need thereof.

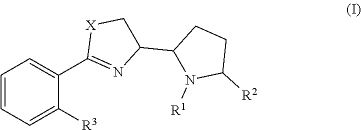

### CROSS-REFERENCE TO RELATED APPLICATIONS

This application claims the benefit of the priority of U.S. Provisional Patent Application No. 63/424,061, filed Nov. 9, 2022, which is incorporated by reference herein.

### TECHNICAL FIELD

The present disclosure provides compounds, pharmaceutical compositions, dosage forms, and methods of treating inflammation, decreasing inflammation, decreasing inflammatory markers, and treating inflammatory bowel disease, such as Crohn's disease or ulcerative colitis.

### BACKGROUND

Compounds that suppress inflammatory immune responses are needed.

### SUMMARY

In some aspects, the disclosure provides compound of Formula (I), or a pharmaceutically acceptable salt thereof:

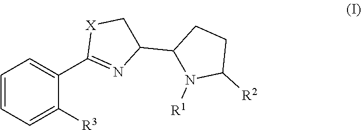

wherein, R¹ to R³ and X are defined herein; with the proviso that the compound is not (2S,5S)-5-((S)-2-(2-hydroxyphenyl)-4,5-dihydrothiazol-4-yl)-1-methylpyrrolidine-2-carboxylic acid or (2S,5S)-5-((S)-2-(2-hydroxyphenyl)-4,5-dihydrooxazol-4-yl)-1-methylpyrrolidine-2-carboxylic acid.

In further aspects, the disclosure provides compounds of Formula (IA) or a pharmaceutically acceptable salt thereof:

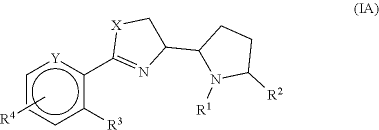

wherein, R¹ to R⁴, X, and Y are defined herein; with the proviso that the compound is not (2S,5S)-5-((S)-2-(2-hydroxyphenyl)-4,5-dihydrothiazol-4-yl)-1-methylpyrrolidine-2-carboxylic acid or (2S,5S)-5-((S)-2-(2-hydroxyphenyl)-4,5-dihydrooxazol-4-yl)-1-methylpyrrolidine-2-carboxylic acid.

In other aspects, the disclosure provides pharmaceutical compositions comprising a pharmaceutically acceptable excipient and a compound of Formula (I) or a pharmaceutically acceptable salt thereof:

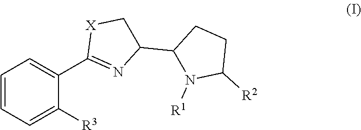

wherein, R¹ to R³ and X are defined herein.

In other aspects, the disclosure provides dosage forms comprising the compounds or pharmaceutical compositions described herein.

In other aspects, the disclosure provides methods of decreasing the level, expression, or activity of an inflammatory marker (e.g., in vitro, ex-vivo or in vivo in a subject), e.g., decreasing the level, expression, or activity of a marker selected from: (i) a cytokine, e.g., an interleukin (IL), e.g., IL-1 (including IL-1a and IL-1β), IL-4, IL-6, IL-8, IL-9, IL-10, IL-11, IL-12, IL-13, IL-17, IL-19, IL-23, IL-35, and IL-36; (ii) tumor necrosis factor alpha (TNF-α); (iii) interferon gamma (IFNγ); (iv) granulocyte-macrophage colony stimulating factor (GM-CSF), (v) a prostaglandin such as prostaglandin E₂ (PGE2); (vi) a chemokine such as GRO-α.

In further aspects, the disclosure provides methods of treating inflammation in a subject in need thereof, comprising administering the compounds, pharmaceutical compositions or dosage forms described herein to the subject in need thereof.

In yet other aspects, the disclosure provides methods of decreasing inflammation in a subject in need thereof, comprising administering the compounds, pharmaceutical compositions or dosage forms to the subject in need thereof.

In still further aspects, the disclosure provides methods of decreasing an inflammatory marker in a subject (e.g., a human) in need thereof, comprising administering the compounds, pharmaceutical compositions or dosage forms described herein to the subject in need thereof.

In other aspects, the disclosure provides methods of treating inflammatory bowel disease, such as Crohn's disease or ulcerative colitis in a subject in need thereof, comprising administering the compounds, pharmaceutical compositions, dosage forms described herein to the subject in need thereof.

In yet other aspects, the disclosure provides methods of treating sepsis in a subject in need thereof, comprising administering the compounds, pharmaceutical compositions, dosage forms described herein to the subject in need thereof.

In further aspects, the disclosure provides probes comprising one or more compounds described herein.

### BRIEF DESCRIPTION OF THE DRAWINGS

FIGS. 1A-1D are line graphs of the IL-23 dose response, IL-6 dose response, IL-12p70 dose response, and TNFa dose response, respectively, for Compound I-4.

FIG. 2 is schematic showing injection of the TLR4 agonist LPS in LPS induced septic shock model in mice.

FIG. 3 is a bar graph comparing the IFNγ levels for Compounds I-3 and I-4 against controls.

FIG. 4 is a bar graph comparing the TFNα levels for Compounds I-3 and I-4 against controls.

FIG. 5 is a bar graph comparing the IL-12p70 for Compounds I-3 and I-4 against controls.

FIG. 6 is a line graph of the change of % initial weight over 7 days for Compound I-3 dosed at day-1 compared to controls.

FIG. 7 is a line graph of the change of % initial weight over 7 days for Compound I-4 dosed at day-1 compared to controls.

FIG. 8 is a line graph of the DAI (Disease Activity Index) score over 7 days for Compound I-3 (1, 3, 10, or 30 mg/kg) compared to controls.

FIG. 9 is a line graph of the DAI score at Day 6 for Compound I-3 at varying dosages compared to controls.

FIG. 10 is a line graph of the DAI score over 7 days for Compound I-4 (1, 3, or 10 mg/kg) compared to controls.

FIG. 11 is a line graph of the DAI score at Day 6 for Compound I-4 at varying dosages compared to controls.

FIG. 12 is a bar graph of the degree of inflammatory cell infiltration for Compound I-3 at varying dosages as compared to controls.

FIG. 13 is a bar graph of the degree of inflammatory cell infiltration for Compound I-4 at varying dosages as compared to controls.

FIG. 14 is a bar graph of the total histology score for Compound I-3 at varying dosages as compared to controls.

FIG. 15 is a bar graph of the total histology score for Compound I-4 at varying dosages as compared to controls.

FIG. 16 are micrographs of H&E-stained colon tissue sections from mice treated with Compound I-3 and Compound I-4.

FIG. 17 are bar graphs of cytokine level (IL-6 or IL1β) for varying concentrations of Compound I-4 as compared to controls.

FIG. 18 is a schematic for the measurement of inflammatory cytokine release in plasma.

FIG. 19 is a line graph for the progression of % initial weight when dosing from day 2 of Compound I-3 compared to controls.

FIG. 20 is a line graph for the progression of % initial weight when dosing from day 2 of Compound I-4 compared to controls.

FIG. 21 is a line graph of the DAI score over 7 days for Compound I-3 (30 mg/kg) compared to controls.

FIG. 22 is a line graph of the DAI score over 7 days for Compound I-4 (30 mg/kg) compared to controls.

FIG. 23 is a bar graph of the pathology score for Compound I-3 and controls.

FIG. 24 is a bar graph of IL1β level for Compounds I-3 and I-4 as compared to controls.

FIG. 25 is a bar graph of IL-6 level for Compounds I-3 and I-4 as compared to controls.

FIG. 26 is a line graph of the DAI score for compound I-86 for days 1-6.

FIG. 27 is a line graph showing the TNBS DAI score for compound I-88 for days 1-8.

FIG. 28 is a bar graph of the DAI score for compound I-88 at Day 6.

### DETAILED DESCRIPTION OF ILLUSTRATIVE EMBODIMENTS

In the disclosure, the singular forms “a”, “an” and “the” include the plural reference, and reference to a particular numerical value includes at least that particular value, unless the context indicates otherwise. For example, reference to “a material” is a reference to at least one of such materials and equivalents thereof known to those skilled in the art, and so forth.

When a value is expressed as an approximation by use of the descriptor “about” it will be understood that the particular value forms another embodiment. In general, use of the term “about” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about”. In other cases, the gradations used in a series of values may be used to determine the intended range available to the term “about” for each value. Where present, all ranges are inclusive and combinable. That is, references to values stated in ranges include every value within that range.

When a list is presented, unless stated otherwise, it is to be understood that each individual element and every combination is to be interpreted as separate embodiments. For example, a list of embodiments presented as “A, B, or C” is to be interpreted as including the embodiments, “A,” “B,” “C,” “A or B,” “A or C,” “B or C,” or “A, B, or C.”

It is to be appreciated that certain features of the invention which are, for clarity, described herein in the context of separate embodiments, may also be provided in combination in a single embodiment. That is, unless obviously incompatible or excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. Finally, while an embodiment may be described as part of a series of steps or part of a more general structure, each said step may also be considered an independent embodiment in itself.

It is noted that the claims may be drafted to exclude an optional element. As such, this statement is intended to serve as antecedent basis for use of such exclusive terminology as “solely,” “only” and the like in connection with the recitation of claim elements, or use of a “negative” limitation.

The terms “subject” and “patient” are used interchangeably and include, without limitation, mammals. In some embodiments, the patient or subject is a human. In other embodiments, the patient or subject is a veterinary or farm animal, a domestic animal or pet, or animal used for clinical research. In certain embodiments, the subject or patient is an adult, i.e., ≥18 years of age. In further embodiments, the subject or patient is a pediatric subject or patient, i.e., <18 years of age.

The term “halo” represents chlorine, fluorine, bromine, or iodine.

The term “alkyl,” as used herein refers to a straight- or branched-chain alkyl groups. In some embodiments, the alkyl group has from 1 to 6 carbons atoms, i.e., C₁₋₆alkyl. Examples of alkyls include methyl (C₁alkyl), ethyl (C₂alkyl), n-propyl (C₃alkyl), isopropyl (C₃alkyl). In some embodiments, the alkyl is methyl. In other embodiments, the alkyl is ethyl. In further embodiments, the alkyl is n-propyl or isopropyl. In still further embodiments, the alkyl is n-butyl, i-butyl, s-butyl, or t-butyl. In other embodiments, the alkyl is pentyl. In further embodiments, the alkyl is hexyl. The alkyl group is optionally substituted with one, two, or three substituents selected from halo, OH, OC₁₋₆alkyl, CN, NH₂, NH(C₁₋₆alkyl), NH(C₁₋₆alkyl)₂, C₃₋₈cycloalkyl, heterocyclyl, or aryl. The alkyl group may be substituted with an optionally substituted diazirinyl group such as diazirinyl, diazirinyl substituted with alkynyl, or -diazirinyl-alkyl-alkynyl.

As used herein, “alkenyl” refers to a straight or branched chain hydrocarbon radical having 2 to 6 carbon atoms that has at least one point of unsaturation, i.e., one double bond. In some embodiments, the number of carbon atoms is designated (i.e., C₂₋₆ means two to six carbons). In some embodiments, the alkenyl is a straight chain hydrocarbon. In other embodiments, the alkenyl is a branched chain hydrocarbon. The alkenyl group has at least one carbon-carbon double bond. In some embodiments, the alkenyl has one double bond. In other embodiments, the alkenyl has two or more double bonds. The alkenyl group may be substituted with one or more group as described herein. An alkenyl is optionally substituted with one, two, or three substituents selected from halo, OH, OC₁₋₆alkyl, CN, NH₂, NH(C₁₋₆alkyl), NH(C₁₋₆alkyl)₂, C₃₋₈cycloalkyl, heterocyclyl, or aryl.

“Cycloalkyl” as used herein refers to a monocyclic, non-aromatic hydrocarbon group. In some embodiments, the cycloalkyl group has from 3 to 8 carbon atoms (“C₃₋₈”). In some embodiments, the cycloalkyl has from 3 to 6 carbon atoms. Examples of cycloalkyl groups include, e.g., cyclopropyl (C₃), cyclobutyl (C₄), cyclopentyl (C₅), 1-methylcyclopropyl (C₄), or 2-methylcyclopentyl (C₄), among others. A cycloalkyl is optionally substituted with one, two, or three substituents selected from halo, OH, OC₁₋₆alkyl, CN, NH₂, NH(C₁₋₆alkyl), NH(C₁₋₆alkyl)₂, C₃₋₈cycloalkyl, heterocyclyl, or aryl.

The term “heterocyclyl” or “heterocycloalkyl” refers to a stable 3- to 14-membered non-aromatic ring radical that comprises two to twelve carbon atoms and from 1 to 6 heteroatoms selected from nitrogen, oxygen and sulfur. In certain embodiments, the heterocycloalkyl is a 4-6 membered ring. In other embodiments, the heterocycloalkyl is a 4-membered ring. In further embodiments, the heterocycloalkyl is a 5-membered ring. In yet other embodiments, the heterocycloalkyl is a 6 membered ring. In some embodiments, the heterocyclyl contains 3 ring atoms, 4 ring atoms, etc., up to and including 14 ring atoms. The heterocyclyl is a monocyclic, bicyclic, tricyclic or tetracyclic ring system, which may include fused or bridged ring systems. The heteroatoms in the heterocyclyl radical may be optionally oxidized. One or more nitrogen atoms, if present, are optionally quaternized. The heterocyclyl radical is unsaturated, partially saturated, or fully saturated. The heterocyclyl may be attached to the rest of the molecule through any atom of the ring(s). “Heterocyclyl” also includes bicyclic ring systems wherein one non-aromatic ring contains at least 2 carbon atoms in addition to 1-3 heteroatoms independently selected from oxygen, sulfur, and nitrogen, as well as combinations comprising at least one of the foregoing heteroatoms; and the other ring optionally contains 1-3 heteroatoms independently selected from oxygen, sulfur, and nitrogen and is not aromatic. In some embodiments, the heterocyclyl is indolyl. Examples of heterocyclyl include, but are not limited to, azepanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl, azepinyl, acridinyl, benzimidazolyl, benzindolyl, 1,3-benzodioxolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzodioxolyl, benzodioxinyl, benzoxazolyl, benzopyranyl, benzopyranonyl, benzofuranyl, benzofuranonyl, benzofurazanyl, benzothiazolyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7] cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a, 7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). A heterocyclyl is optionally substituted with one, two, or three substituents selected from halo, OH, OC₁₋₆alkyl, CN, NH₂, NH(C₁₋₆alkyl), NH(C₁₋₆alkyl)₂, C₃₋₈cycloalkyl, heterocyclyl, or aryl.

The term “aryl” refers to carbocyclic aromatic groups having from 6 to 10 carbon atoms (“C₆₋₁₀”) such as phenyl, naphthyl, and the like. An aryl is optionally substituted with one, two, or three substituents selected from halo, OH, OC₁₋₆alkyl, CN, NH₂, NH(C₁₋₆alkyl), NH(C₁₋₆alkyl)₂, C₃₋₈cycloalkyl, heterocyclyl, or aryl.

The term “biotinylated” used herein refers a compound described herein comprising one or more biotinyl groups. Biotinylation may be performed using skill and techniques known in the art. The biotinyl group may bound directly to the compound or may be bound through a linker. In certain aspects, the linker is —C(O)—, —C(O)NH—, —(CH₂CH₂O)_{z}—, or —(CH₂CH₂O)_{z}(CH₂)_{y}—, or a combination thereof, wherein z is 1-10 and y is 1-5.

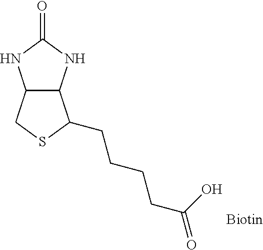 [SMILES: O=C(O)CCCCC1SCC2NC(=O)NC21]

### Compounds

The present disclosure provides compounds of Formula (I) or (IA) or a pharmaceutically acceptable salt thereof:

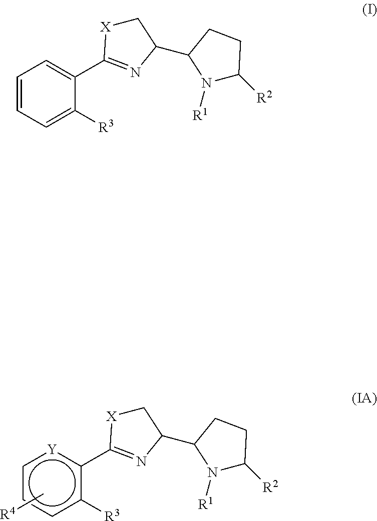

In some embodiments, the compound is not (2S,5S)-5-((S)-2-(2-hydroxyphenyl)-4,5-dihydrothiazol-4-yl)-1-methylpyrrolidine-2-carboxylic acid:

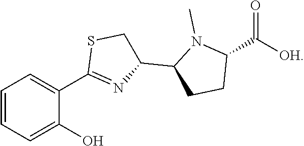 [SMILES: CN1[C@H](C(=O)O)CC[C@H]1[C@H]1CSC(c2ccccc2O)=N1]

In other embodiments, the compound is not (2S,5S)-5-((S)-2-(2-hydroxyphenyl)-4,5-dihydrooxazol-4-yl)-1-methylpyrrolidine-2-carboxylic acid:

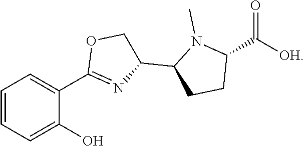 [SMILES: CN1[C@H](C(=O)O)CC[C@H]1[C@H]1COC(c2ccccc2O)=N1]

According to the disclosure, R¹ is C₁₋₆alkyl or C₂₋₆alkenyl. In some embodiments, R¹ is C₁₋₆alkyl. In further embodiments, R¹ is C₁alkyl, C₂alkyl, C₃alkyl, C₄alkyl, C₅alkyl, or C₆alkyl. In other embodiments, R¹ is CH₃. In further embodiments, R¹ is CH₂CH₃. In yet other embodiments, R¹ is propyl, such as n-propyl or i-propyl. In still further embodiments, R¹ is butyl, such as s-butyl, i-butyl, n-butyl, or t-butyl. In other embodiments, R¹ is pentyl. In further embodiments, R¹ is hexyl. In yet other embodiments, R¹ is C₂alkenyl, C₃alkenyl, C₄alkenyl, C₅alkenyl, or C₆alkenyl. In still further embodiments, R¹ is CH—CH₂. In yet other embodiments, R¹ is propenyl, such as n-propenyl or i-propenyl. In still further embodiments, R¹ is butenyl, such as s-butenyl, i-butenyl, n-butenyl, or t-butenyl. In other embodiments, R¹ is pentenyl. In further embodiments, R¹ is hexenyl.

According to the disclosure, R² is H, C(O)OH, or a masked carboxylic acid. In some embodiments, R² is H. In other embodiments, R² is C(O)OH. As used herein, the term “masked carboxylic acid” refers to any chemical moiety that can be converted to a carboxylic acid (COOH) functional group through one or more chemical transformations. Examples of masked carboxylic acids include, e.g., esters, thioesters, oxazoles, nitriles, amides, alkyl alcohols, phenyls, ethers, and orthoesters. In further embodiments, R² is a masked carboxylic acid. In still other embodiments, R² is a masked carboxylic that is —C(O)OC₁₋₆alkyl, —C(O)SC₁₋₆alkyl, oxazolyl, CN, —C(O)NH₂, —C(O)NHR^{A}, —C(O)N(R^{A})₂, —C(O)OC₁₋₆alkylOH, or —C(O)Ophenyl, wherein each R^{A} is, independently, C₁₋₆alkyl, C₂₋₆alkynyl, cycloalkyl, aryl such as phenyl, —CH₂OH, —CH₂OC₁₋₆alkyl, —C(OC₁₋₆alkyl)₃, or (C₁₋₆alkyl-O)ₙ—(C₁₋₆alkyl)-NH-biotinyl (n is 1-6). In other embodiments, two R^{A}, together with the atoms to which they are attached, are joined together to form a heterocycloalkyl, such as a 4-6 membered heterocycloalkyl, or a 4-membered heterocycloalkyl, or a 5-membered heterocycloalkyl, or a 6-membered heterocycloalkyl. In yet further embodiments, R² is —C(O)OC₁₋₆alkyl. In other embodiments, R² is —C(O)SC₁₋₆alkyl. In further embodiments, R² is oxazolyl. In yet other embodiments, R² is CN. In other embodiments, R² is —C(O)NH₂. In further embodiments, R² is —C(O)NHR^{A}, wherein R^{A} is C₁₋₆alkyl, cycloalkyl, aryl, —CH₂OH, phenyl, —CH₂OC₁₋₆alkyl, —C(OC₁₋₆alkyl)₃, or (C₁₋₆alkyl-O)ₙ—(C₁₋₆alkyl)-NH-biotinyl (n is 1-6, such as 1, or such as 2, or such as 3, or such as 4, or such as 5, or such as 6). In still other embodiments, R² is —C(O)N(R^{A})₂, wherein each R^{A} is, independently, C₁₋₆alkyl, cycloalkyl, aryl, —CH₂OH, phenyl, —CH₂OC₁₋₆alkyl, —C(OC₁₋₆alkyl)₃, or (C₁₋₆alkyl-O)ₙ—(C₁₋₆alkyl)-NH-biotinyl (n is 1-6, such as 1, or such as 2, or such as 3, or such as 4, or such as 5, or such as 6). In yet further embodiments, R² is —C(O)OC₁₋₆alkylOH. In other embodiments, R² is —C(O)Ophenyl. In further embodiments, R² is biotinylated. In yet other embodiments, R² is a biotinylated masked carboxylic acid such as —C(O)NH—(C₁₋₆alkyl-O)ₙ—(C₁₋₆alkyl)-NH-biotinyl (n is 1-6, such as 1, or such as 2, or such as 3, or such as 4, or such as 5, or such as 6).

According to the disclosure, R³ is H, OH, or OSO₂Z, wherein Z is halo. In some embodiments, R³ is H. In other embodiments, R³ is OH. In further embodiments, R³ is OSO₂Z. In some aspects, Z is Cl, F, Br, or I. In other aspects, Z is F. In further aspects, Z is Cl.

According to the disclosure, R⁴ is H or O—(C₁₋₆alkyl-O) n-C₁₋₆alkyl or —(C₁₋₆alkyl-O) n-H; and n is 1-4. In some embodiments, R⁴ is H. In other embodiments, R⁴ is —O—(CH₂CH₂)ₙ—C₁₋₆alkyl, such as —O—(CH₂CH₂)—O—(CH₂CH₂)—O—CH₃. In further embodiments, n is 1. In yet other embodiments, n is 2. In still further embodiments, n is 3. In other embodiments, n is 4.

According to the disclosure, X is O or S. In some embodiments, X is O. In other embodiments, X is S.

According to the disclosure, Y is N or CH. In some embodiments, Y is N. In other embodiments, Y is CH.

In certain embodiments, the compound is of Formula (I). In other embodiments, the compound is of Formula (IA).

In certain embodiments, the compound is of formula (II), wherein X, R¹, and R² are defined herein:

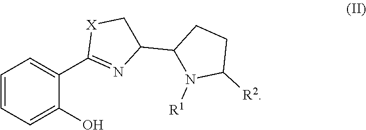

In other embodiments, the compound is of formula (III), wherein X, R¹, and R² are defined herein:

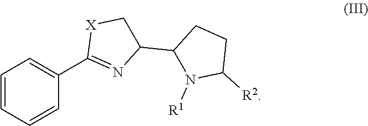

In further embodiments, the compound is of formula (IV), wherein X and R¹ are defined herein:

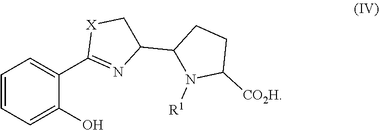

In still other embodiments, the compound is of formula (V), wherein X and R¹ are defined herein:

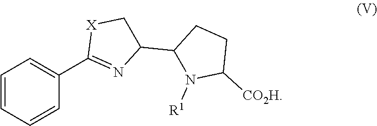

In yet further embodiments, the compound is of formula (VI), wherein X and R¹—R³ are defined herein:

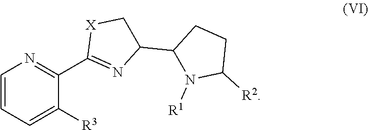

In yet further embodiments, the compound is of formula (VII), wherein X, Y, R¹, and R² are defined herein:

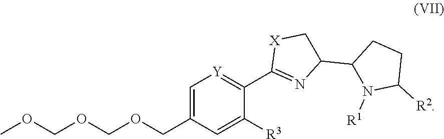

In other embodiments, the compound is:

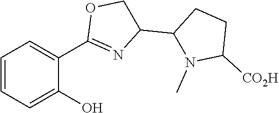 [SMILES: CN1C(C(=O)O)CCC1C1COC(c2ccccc2O)=N1]

Compound A (5-(2-(2-hydroxyphenyl)-4,5-dihydrooxazol-4-yl)-1-methylpyrrolidine-2-carboxylic acid), such as:

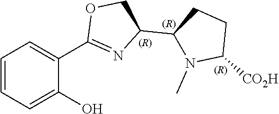

Compound I-1 ((2R,5R)-5-((R)-2-(2-hydroxyphenyl)-4,5-dihydrooxazol-4-yl)-1-methylpyrrolidine-2-carboxylic acid); or

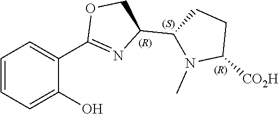

Compound I-2 ((2R,5S)-5-((R)-2-(2-hydroxyphenyl)-4,5-dihydrooxazol-4-yl)-1-methylpyrrolidine-2-carboxylic acid).

In further embodiments, the compound is:

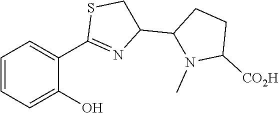 [SMILES: CN1C(C(=O)O)CCC1C1CSC(c2ccccc2O)=N1]

Compound B (5-(2-(2-hydroxyphenyl)-4,5-dihydrothiazol-4-yl)-1-methylpyrrolidine-2-carboxylic acid), such as:

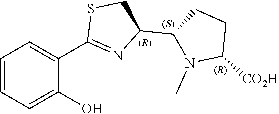

Compound I-4 ((2R,5S)-5-((R)-2-(2-hydroxyphenyl)-4,5-dihydrothiazol-4-yl)-1-methylpyrrolidine-2-carboxylic acid);

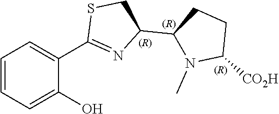

Compound I-3 ((2R,5R)-5-((R)-2-(2-hydroxyphenyl)-4,5-dihydrothiazol-4-yl)-1-methylpyrrolidine-2-carboxylic acid); or

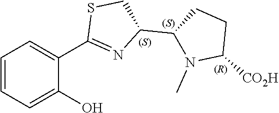

Compound I-12 ((2R,5S)-5-((S)-2-(2-hydroxyphenyl)-4,5-dihydrothiazol-4-yl)-1-methylpyrrolidine-2-carboxylic acid).

In further embodiments, the compound is:

| Compound # | Structure |
|---|---|
| C | 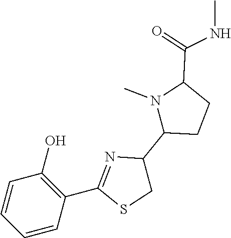 [SMILES: CNC(=O)C1CCC(C2CSC(c3ccccc3O)=N2)N1C] |
| D | 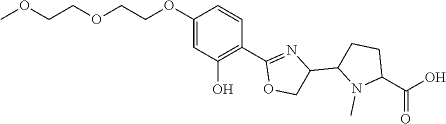 [SMILES: COCCOCCOc1ccc(C2=NC(C3CCC(C(=O)O)N3C)CO2)c(O)c1] |
| E | 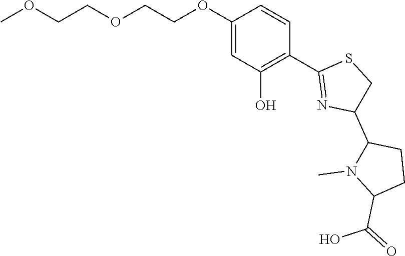 [SMILES: COCCOCCOc1ccc(C2=NC(C3CCC(C(=O)O)N3C)CS2)c(O)c1] |
| F | 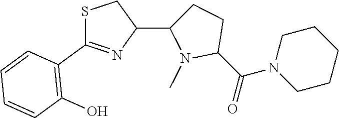 [SMILES: CN1C(C(=O)N2CCCCC2)CCC1C1CSC(c2ccccc2O)=N1] |
| G | 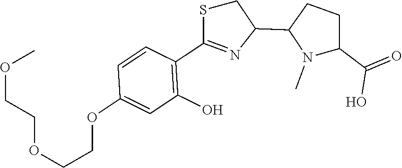 [SMILES: COCCOCCOc1ccc(C2=NC(C3CCC(C(=O)O)N3C)CS2)c(O)c1] |
| H | 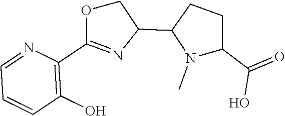 [SMILES: CN1C(C(=O)O)CCC1C1COC(c2ncccc2O)=N1] |
| J | 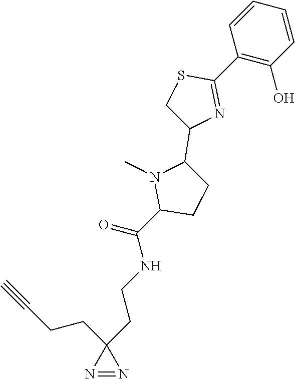 [SMILES: C#CCCC1(CCNC(=O)C2CCC(C3CSC(c4ccccc4O)=N3)N2C)N=N1] |
| K | 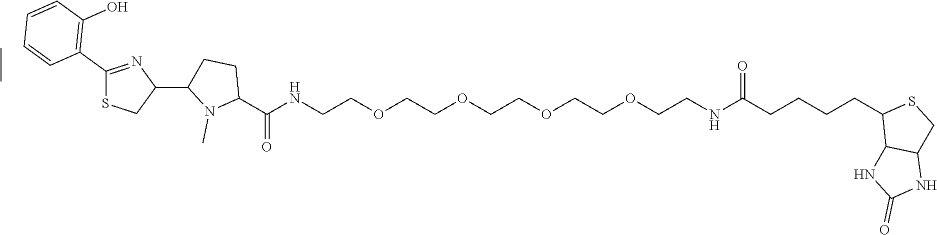 |
| L | 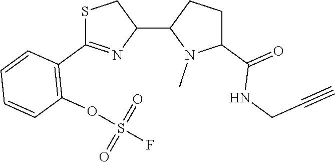 [SMILES: C#CCNC(=O)C1CCC(C2CSC(c3ccccc3OS(=O)(=O)F)=N2)N1C] |

or a pharmaceutically acceptable salt thereof.

In yet other embodiments, the compound is:

| Compound # | Structure |
|---|---|
| I-5 | 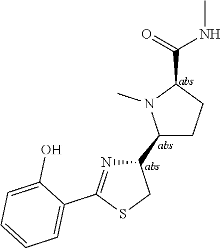 [SMILES: CNC(=O)[C@H]1CC[C@@H]([C@@H]2CSC(c3ccccc3O)=N2)N1C] |
| I-6 | 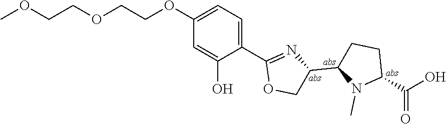 [SMILES: COCCOCCOc1ccc(C2=N[C@H]([C@H]3CC[C@H](C(=O)O)N3C)CO2)c(O)c1] |
| I-7 | 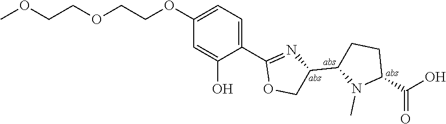 [SMILES: COCCOCCOc1ccc(C2=N[C@H]([C@@H]3CC[C@H](C(=O)O)N3C)CO2)c(O)c1] |
| I-8 | 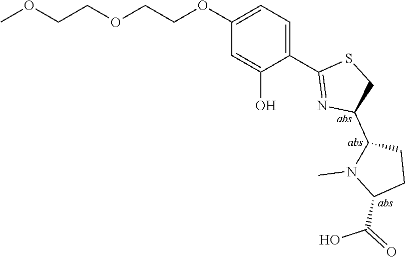 [SMILES: COCCOCCOc1ccc(C2=N[C@H]([C@@H]3CC[C@H](C(=O)O)N3C)CS2)c(O)c1] |
| I-9 | 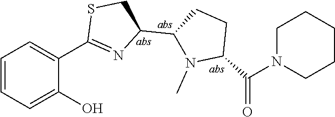 [SMILES: CN1[C@@H](C(=O)N2CCCCC2)CC[C@H]1[C@@H]1CSC(c2ccccc2O)=N1] |
| I-10 | 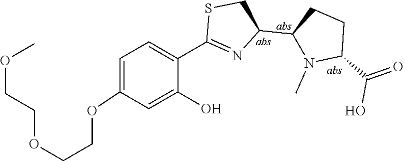 [SMILES: COCCOCCOc1ccc(C2=N[C@H]([C@H]3CC[C@H](C(=O)O)N3C)CS2)c(O)c1] |
| I-11 | 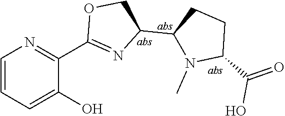 [SMILES: CN1[C@@H](C(=O)O)CC[C@@H]1[C@@H]1COC(c2ncccc2O)=N1] |
| I-13 | 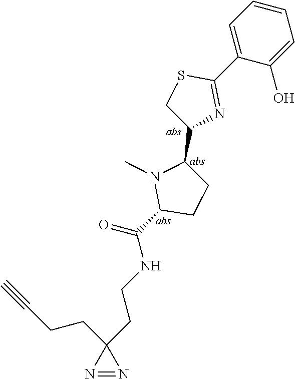 [SMILES: C#CCCC1(CCNC(=O)[C@H]2CC[C@H]([C@@H]3CSC(c4ccccc4O)=N3)N2C)N=N1] |
| I-14 | 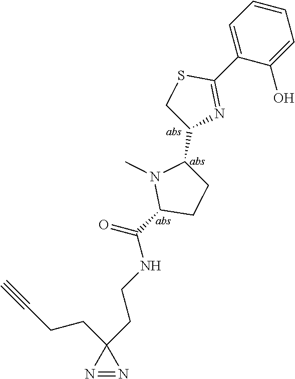 [SMILES: C#CCCC1(CCNC(=O)[C@H]2CC[C@@H]([C@@H]3CSC(c4ccccc4O)=N3)N2C)N=N1] |
| I-15 | 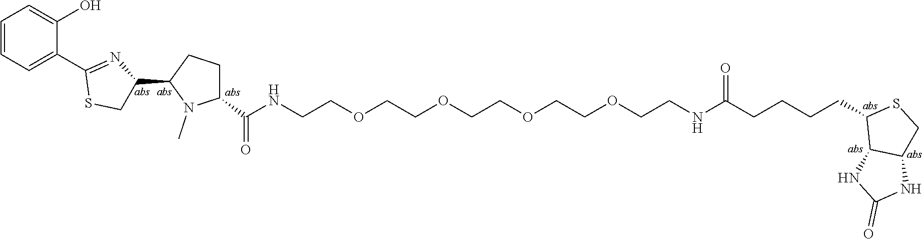 [SMILES: CN1[C@@H](C(=O)NCCOCCOCCOCCOCCNC(=O)CCCC[C@@H]2SC[C@@H]3NC(=O)N[C@@H]32)CC[C@@H]1[C@@H]1CSC(c2ccccc2O)=N1] |
| I-16 | 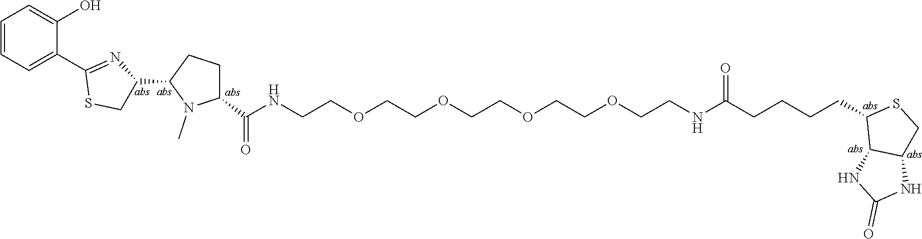 [SMILES: CN1[C@@H](C(=O)NCCOCCOCCOCCOCCNC(=O)CCCC[C@@H]2SC[C@@H]3NC(=O)N[C@@H]32)CC[C@H]1[C@@H]1CSC(c2ccccc2O)=N1] |
| I-17 | 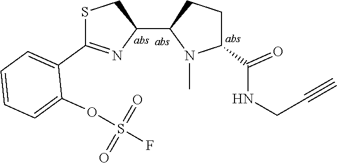 [SMILES: C#CCNC(=O)[C@H]1CC[C@H]([C@@H]2CSC(c3ccccc3OS(=O)(=O)F)=N2)N1C] |
| I-18 | 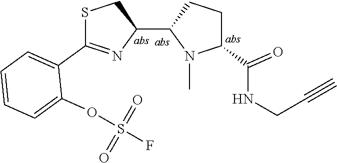 [SMILES: C#CCNC(=O)[C@H]1CC[C@@H]([C@@H]2CSC(c3ccccc3OS(=O)(=O)F)=N2)N1C] |

or a pharmaceutically acceptable salt thereof.

In still further embodiments, the compound is:

| Compound # | Name |
|---|---|
| I-19A | (2R,5R)-5-((S)-2-(2-hydroxyphenyl)-4,5-dihydrooxazol-4-yl)-1- |
| | methylpyrrolidine-2-carboxylic acid. |
| I-19B | (2R,5S)-5-((S)-2-(2-hydroxyphenyl)-4,5-dihydrooxazol-4-yl)-1- |
| | methylpyrrolidine-2-carboxylic acid. |
| I-19C | (2S,5R)-5-((R)-2-(2-hydroxyphenyl)-4,5-dihydrooxazol-4-yl)-1- |
| | methylpyrrolidine-2-carboxylic acid. |
| I-19D | (2S,5R)-5-((S)-2-(2-hydroxyphenyl)-4,5-dihydrooxazol-4-yl)-1- |
| | methylpyrrolidine-2-carboxylic acid. |
| I-19E | (2S,5S)-5-((R)-2-(2-hydroxyphenyl)-4,5-dihydrooxazol-4-yl)-1- |
| | methylpyrrolidine-2-carboxylic acid. |
| I-19F | (2S,5S)-5-((S)-2-(2-hydroxyphenyl)-4,5-dihydrooxazol-4-yl)-1- |
| | methylpyrrolidine-2-carboxylic acid. |
| I-20A | (2R,5R)-5-((S)-2-(2-hydroxyphenyl)-4,5-dihydrothiazol-4-yl)-1- |
| | methylpyrrolidine-2-carboxylic acid |
| I-20B | (2S,5R)-5-((R)-2-(2-hydroxyphenyl)-4,5-dihydrothiazol-4-yl)-1- |
| | methylpyrrolidine-2-carboxylic acid |
| I-20C | (2S,5R)-5-((S)-2-(2-hydroxyphenyl)-4,5-dihydrothiazol-4-yl)-1- |
| | methylpyrrolidine-2-carboxylic acid |
| I-20D | (2S,5S)-5-((R)-2-(2-hydroxyphenyl)-4,5-dihydrothiazol-4-yl)-1- |
| | methylpyrrolidine-2-carboxylic acid |
| I-20E | (2S,5S)-5-((S)-2-(2-hydroxyphenyl)-4,5-dihydrothiazol-4-yl)-1- |
| | methylpyrrolidine-2-carboxylic acid |
| I-21A | (2R,5R)-5-((R)-2-(2-hydroxyphenyl)-4,5-dihydrothiazol-4-yl)-N,1-dimethyl |
| | pyrrolidine-2-carboxamide |
| I-21B | (2R,5R)-5-((S)-2-(2-hydroxyphenyl)-4,5-dihydrothiazol-4-yl)-N,1-dimethyl |
| | pyrrolidine-2-carboxamide |
| I-21C | (2R,5S)-5-((S)-2-(2-hydroxyphenyl)-4,5-dihydrothiazol-4-yl)-N,1-dimethyl |
| | pyrrolidine-2-carboxamide |
| I-21D | (2S,5R)-5-((R)-2-(2-hydroxyphenyl)-4,5-dihydrothiazol-4-yl)-N,1-dimethyl |
| | pyrrolidine-2-carboxamide |
| I-21E | (2S,5R)-5-((S)-2-(2-hydroxyphenyl)-4,5-dihydrothiazol-4-yl)-N,1-dimethyl |
| | pyrrolidine-2-carboxamide |
| I-21F | (2S,5S)-5-((R)-2-(2-hydroxyphenyl)-4,5-dihydrothiazol-4-yl)-N,1-dimethyl |
| | pyrrolidine-2-carboxamide |
| I-21G | (2S,5S)-5-((S)-2-(2-hydroxyphenyl)-4,5-dihydrothiazol-4-yl)-N,1-dimethyl |
| | pyrrolidine-2-carboxamide |
| I-22A | (2R,5R)-5-((S)-2-(2-hydroxy-4-(2-(2-methoxyethoxy)ethoxy)phenyl)-4,5- |
| | dihydrooxazol-4-yl)-1-methylpyrrolidine-2-carboxylic acid |
| I-22B | (2R,5S)-5-((R)-2-(2-hydroxy-4-(2-(2-methoxyethoxy)ethoxy)phenyl)-4,5- |
| | dihydrooxazol-4-yl)-1-methylpyrrolidine-2-carboxylic acid |
| I-22C | (2R,5S)-5-((S)-2-(2-hydroxy-4-(2-(2-methoxyethoxy)ethoxy)phenyl)-4,5- |
| | dihydrooxazol-4-yl)-1-methylpyrrolidine-2-carboxylic acid |
| I-22D | (2S,5R)-5-((R)-2-(2-hydroxy-4-(2-(2-methoxyethoxy)ethoxy)phenyl)-4,5- |
| | dihydrooxazol-4-yl)-1-methylpyrrolidine-2-carboxylic acid |
| I-22E | (2S,5R)-5-((S)-2-(2-hydroxy-4-(2-(2-methoxyethoxy)ethoxy)phenyl)-4,5- |
| | dihydrooxazol-4-yl)-1-methylpyrrolidine-2-carboxylic acid |
| I-22F | (2S,5S)-5-((R)-2-(2-hydroxy-4-(2-(2-methoxyethoxy)ethoxy)phenyl)-4,5- |
| | dihydrooxazol-4-yl)-1-methylpyrrolidine-2-carboxylic acid |
| I-22G | (2S,5S)-5-((S)-2-(2-hydroxy-4-(2-(2-methoxyethoxy)ethoxy)phenyl)-4,5- |
| | dihydrooxazol-4-yl)-1-methylpyrrolidine-2-carboxylic acid |
| I-23A | (2R,5R)-5-((R)-2-(2-hydroxy-4-(2-(2-methoxyethoxy)ethoxy)phenyl)-4,5- |
| | dihydrooxazol-4-yl)-1-methylpyrrolidine-2-carboxylic acid |
| I-23B | (2R,5R)-5-((S)-2-(2-hydroxy-4-(2-(2-methoxyethoxy)ethoxy)phenyl)-4,5- |
| | dihydrooxazol-4-yl)-1-methylpyrrolidine-2-carboxylic acid |
| I-23C | (2R,5S)-5-((S)-2-(2-hydroxy-4-(2-(2-methoxyethoxy)ethoxy)phenyl)-4,5- |
| | dihydrooxazol-4-yl)-1-methylpyrrolidine-2-carboxylic acid |
| I-23D | (2S,5R)-5-((R)-2-(2-hydroxy-4-(2-(2-methoxyethoxy)ethoxy)phenyl)-4,5- |
| | dihydrooxazol-4-yl)-1-methylpyrrolidine-2-carboxylic acid |
| I-23E | (2S,5R)-5-((S)-2-(2-hydroxy-4-(2-(2-methoxyethoxy)ethoxy)phenyl)-4,5- |
| | dihydrooxazol-4-yl)-1-methylpyrrolidine-2-carboxylic acid |
| I-23F | (2S,5S)-5-((R)-2-(2-hydroxy-4-(2-(2-methoxyethoxy)ethoxy)phenyl)-4,5- |
| | dihydrooxazol-4-yl)-1-methylpyrrolidine-2-carboxylic acid |
| I-23G | (2S,5S)-5-((S)-2-(2-hydroxy-4-(2-(2-methoxyethoxy)ethoxy)phenyl)-4,5- |
| | dihydrooxazol-4-yl)-1-methylpyrrolidine-2-carboxylic acid |
| I-24A | (2R,5R)-5-((R)-2-(2-hydroxy-4-(2-(2-methoxyethoxy)ethoxy) phenyl)-4,5- |
| | dihydrothiazol-4-yl)-1-methylpyrrolidine-2-carboxylic acid |
| I-24B | (2R,5R)-5-((S)-2-(2-hydroxy-4-(2-(2-methoxyethoxy)ethoxy) phenyl)-4,5- |
| | dihydrothiazol-4-yl)-1-methylpyrrolidine-2-carboxylic acid |
| I-24C | (2R,5S)-5-((S)-2-(2-hydroxy-4-(2-(2-methoxyethoxy)ethoxy) phenyl)-4,5- |
| | dihydrothiazol-4-yl)-1-methylpyrrolidine-2-carboxylic acid |
| I-24D | (2S,5R)-5-((R)-2-(2-hydroxy-4-(2-(2-methoxyethoxy)ethoxy) phenyl)-4,5- |
| | dihydrothiazol-4-yl)-1-methylpyrrolidine-2-carboxylic acid |
| I-24E | (2S,5R)-5-((S)-2-(2-hydroxy-4-(2-(2-methoxyethoxy)ethoxy) phenyl)-4,5- |
| | dihydrothiazol-4-yl)-1-methylpyrrolidine-2-carboxylic acid |
| I-24F | (2S,5S)-5-((R)-2-(2-hydroxy-4-(2-(2-methoxyethoxy)ethoxy) phenyl)-4,5- |
| | dihydrothiazol-4-yl)-1-methylpyrrolidine-2-carboxylic acid |
| I-24G | (2S,5S)-5-((S)-2-(2-hydroxy-4-(2-(2-methoxyethoxy)ethoxy) phenyl)-4,5- |
| | dihydrothiazol-4-yl)-1-methylpyrrolidine-2-carboxylic acid |
| I-25A | ((2R,5R)-5-((R)-2-(2-hydroxyphenyl)-4,5-dihydrothiazol-4-yl)-1- |
| | methylpyrrolidin-2-yl)(piperidin-1-yl) |
| I-25B | ((2R,5R)-5-((S)-2-(2-hydroxyphenyl)-4,5-dihydrothiazol-4-yl)-1- |
| | methylpyrrolidin-2-yl)(piperidin-1-yl) |
| I-25C | ((2R,5S)-5-((S)-2-(2-hydroxyphenyl)-4,5-dihydrothiazol-4-yl)-1- |
| | methylpyrrolidin-2-yl)(piperidin-1-yl) |
| I-25D | ((2S,5R)-5-((R)-2-(2-hydroxyphenyl)-4,5-dihydrothiazol-4-yl)-1- |
| | methylpyrrolidin-2-yl)(piperidin-1-yl) |
| I-25E | ((2S,5R)-5-((S)-2-(2-hydroxyphenyl)-4,5-dihydrothiazol-4-yl)-1- |
| | methylpyrrolidin-2-yl)(piperidin-1-yl) |
| I-25F | ((2S,5S)-5-((R)-2-(2-hydroxyphenyl)-4,5-dihydrothiazol-4-yl)-1- |
| | methylpyrrolidin-2-yl)(piperidin-1-yl) |
| I-25G | ((2S,5S)-5-((S)-2-(2-hydroxyphenyl)-4,5-dihydrothiazol-4-yl)-1- |
| | methylpyrrolidin-2-yl)(piperidin-1-yl) |
| I-26A | (2R,5R)-5-((S)-2-(2-hydroxy-4-(2-(2-methoxyethoxy)ethoxy)phenyl)-4,5- |
| | dihydrothiazol-4-yl)-1-methylpyrrolidine-2-carboxylic acid |
| I-26B | (2R,5S)-5-((R)-2-(2-hydroxy-4-(2-(2-methoxyethoxy)ethoxy)phenyl)-4,5- |
| | dihydrothiazol-4-yl)-1-methylpyrrolidine-2-carboxylic acid |
| I-26C | (2R,5S)-5-((S)-2-(2-hydroxy-4-(2-(2-methoxyethoxy)ethoxy)phenyl)-4,5- |
| | dihydrothiazol-4-yl)-1-methylpyrrolidine-2-carboxylic acid |
| I-26D | (2S,5R)-5-((R)-2-(2-hydroxy-4-(2-(2-methoxyethoxy)ethoxy)phenyl)-4,5- |
| | dihydrothiazol-4-yl)-1-methylpyrrolidine-2-carboxylic acid |
| I-26E | (2S,5R)-5-((S)-2-(2-hydroxy-4-(2-(2-methoxyethoxy)ethoxy)phenyl)-4,5- |
| | dihydrothiazol-4-yl)-1-methylpyrrolidine-2-carboxylic acid |
| I-26F | (2S,5S)-5-((R)-2-(2-hydroxy-4-(2-(2-methoxyethoxy)ethoxy)phenyl)-4,5- |
| | dihydrothiazol-4-yl)-1-methylpyrrolidine-2-carboxylic acid |
| I-26G | (2S,5S)-5-((S)-2-(2-hydroxy-4-(2-(2-methoxyethoxy)ethoxy)phenyl)-4,5- |
| | dihydrothiazol-4-yl)-1-methylpyrrolidine-2-carboxylic acid |
| I-27A | (2R,5R)-5-((S)-2-(3-hydroxypyridin-2-yl)-4,5-dihydrooxazol-4-yl)-1- |
| | methylpyrrolidine-2-carboxylic acid |
| I-27B | (2R,5S)-5-((R)-2-(3-hydroxypyridin-2-yl)-4,5-dihydrooxazol-4-yl)-1- |
| | methylpyrrolidine-2-carboxylic acid |
| I-27C | (2R,5S)-5-((S)-2-(3-hydroxypyridin-2-yl)-4,5-dihydrooxazol-4-yl)-1- |
| | methylpyrrolidine-2-carboxylic acid |
| I-27D | (2S,5R)-5-((R)-2-(3-hydroxypyridin-2-yl)-4,5-dihydrooxazol-4-yl)-1- |
| | methylpyrrolidine-2-carboxylic acid |
| I-27E | (2S,5R)-5-((S)-2-(3-hydroxypyridin-2-yl)-4,5-dihydrooxazol-4-yl)-1- |
| | methylpyrrolidine-2-carboxylic acid |
| I-27F | (2S,5S)-5-((R)-2-(3-hydroxypyridin-2-yl)-4,5-dihydrooxazol-4-yl)-1- |
| | methylpyrrolidine-2-carboxylic acid |
| I-27G | (2S,5S)-5-((S)-2-(3-hydroxypyridin-2-yl)-4,5-dihydrooxazol-4-yl)-1- |
| | methylpyrrolidine-2-carboxylic acid |
| I-28A | (2R,5R)-N-(2-(3-(but-3-yn-1-yl)-3H-diazirin-3-yl)ethyl)-5-((S)-2-(2- |
| | hydroxyphenyl)-4,5-dihydrothiazol-4-yl)-1-methylpyrrolidine-2-carboxamide |
| I-28B | (2R,5S)-N-(2-(3-(but-3-yn-1-yl)-3H-diazirin-3-yl)ethyl)-5-((R)-2-(2- |
| | hydroxyphenyl)-4,5-dihydrothiazol-4-yl)-1-methylpyrrolidine-2-carboxamide |
| I-28C | (2R,5S)-N-(2-(3-(but-3-yn-1-yl)-3H-diazirin-3-yl)ethyl)-5-((S)-2-(2- |
| | hydroxyphenyl)-4,5-dihydrothiazol-4-yl)-1-methylpyrrolidine-2-carboxamide |
| I-28D | (2S,5R)-N-(2-(3-(but-3-yn-1-yl)-3H-diazirin-3-yl)ethyl)-5-((R)-2-(2- |
| | hydroxyphenyl)-4,5-dihydrothiazol-4-yl)-1-methylpyrrolidine-2-carboxamide |
| I-28E | (2S,5R)-N-(2-(3-(but-3-yn-1-yl)-3H-diazirin-3-yl)ethyl)-5-((S)-2-(2- |
| | hydroxyphenyl)-4,5-dihydrothiazol-4-yl)-1-methylpyrrolidine-2-carboxamide |
| I-28F | (2S,5S)-N-(2-(3-(but-3-yn-1-yl)-3H-diazirin-3-yl)ethyl)-5-((R)-2-(2- |
| | hydroxyphenyl)-4,5-dihydrothiazol-4-yl)-1-methylpyrrolidine-2-carboxamide |
| I-28G | (2S,5S)-N-(2-(3-(but-3-yn-1-yl)-3H-diazirin-3-yl)ethyl)-5-((S)-2-(2- |
| | hydroxyphenyl)-4,5-dihydrothiazol-4-yl)-1-methylpyrrolidine-2-carboxamide |
| I-29A | (2R,5R)-N-(2-(3-(but-3-yn-1-yl)-3H-diazirin-3-yl)ethyl)-5-((R)-2-(2- |
| | hydroxyphenyl)-4,5-dihydrothiazol-4-yl)-1-methylpyrrolidine-2-carboxamide |
| I-29B | (2R,5R)-N-(2-(3-(but-3-yn-1-yl)-3H-diazirin-3-yl)ethyl)-5-((S)-2-(2- |
| | hydroxyphenyl)-4,5-dihydrothiazol-4-yl)-1-methylpyrrolidine-2-carboxamide |
| I-29C | (2R,5S)-N-(2-(3-(but-3-yn-1-yl)-3H-diazirin-3-yl)ethyl)-5-((S)-2-(2- |
| | hydroxyphenyl)-4,5-dihydrothiazol-4-yl)-1-methylpyrrolidine-2-carboxamide |
| I-29D | (2S,5R)-N-(2-(3-(but-3-yn-1-yl)-3H-diazirin-3-yl)ethyl)-5-((R)-2-(2- |
| | hydroxyphenyl)-4,5-dihydrothiazol-4-yl)-1-methylpyrrolidine-2-carboxamide |
| I-29E | (2S,5R)-N-(2-(3-(but-3-yn-1-yl)-3H-diazirin-3-yl)ethyl)-5-((S)-2-(2- |
| | hydroxyphenyl)-4,5-dihydrothiazol-4-yl)-1-methylpyrrolidine-2-carboxamide |
| I-29F | (2S,5S)-N-(2-(3-(but-3-yn-1-yl)-3H-diazirin-3-yl)ethyl)-5-((R)-2-(2- |
| | hydroxyphenyl)-4,5-dihydrothiazol-4-yl)-1-methylpyrrolidine-2-carboxamide |
| I-29G | (2S,5S)-N-(2-(3-(but-3-yn-1-yl)-3H-diazirin-3-yl)ethyl)-5-((S)-2-(2- |
| | hydroxyphenyl)-4,5-dihydrothiazol-4-yl)-1-methylpyrrolidine-2-carboxamide |
| I-30A | (2R,5R)-5-((S)-2-(2-hydroxyphenyl)-4,5-dihydrothiazol-4-yl)-1-methyl-N-(16- |
| | oxo-20-((3aS,4S,6aR)-2-oxohexahydro-1H-thieno[3,4-d]imidazol-4-yl)- |
| | 3,6,9,12-tetraoxa-15-azaicosyl)pyrrolidine-2-carboxamide |
| I-30B | (2R,5S)-5-((R)-2-(2-hydroxyphenyl)-4,5-dihydrothiazol-4-yl)-1-methyl-N-(16- |
| | oxo-20-((3aS,4S,6aR)-2-oxohexahydro-1H-thieno[3,4-d]imidazol-4-yl)- |
| | 3,6,9,12-tetraoxa-15-azaicosyl)pyrrolidine-2-carboxamide |
| I-30C | (2R,5S)-5-((S)-2-(2-hydroxyphenyl)-4,5-dihydrothiazol-4-yl)-1-methyl-N-(16- |
| | oxo-20-((3aS,4S,6aR)-2-oxohexahydro-1H-thieno[3,4-d]imidazol-4-yl)- |
| | 3,6,9,12-tetraoxa-15-azaicosyl)pyrrolidine-2-carboxamide |
| I-30D | (2S,5R)-5-((R)-2-(2-hydroxyphenyl)-4,5-dihydrothiazol-4-yl)-1-methyl-N-(16- |
| | oxo-20-((3aS,4S,6aR)-2-oxohexahydro-1H-thieno[3,4-d]imidazol-4-yl)- |
| | 3,6,9,12-tetraoxa-15-azaicosyl)pyrrolidine-2-carboxamide |
| I-30E | (2S,5R)-5-((S)-2-(2-hydroxyphenyl)-4,5-dihydrothiazol-4-yl)-1-methyl-N-(16- |
| | oxo-20-((3aS,4S,6aR)-2-oxohexahydro-1H-thieno[3,4-d]imidazol-4-yl)- |
| | 3,6,9,12-tetraoxa-15-azaicosyl)pyrrolidine-2-carboxamide |
| I-30F | (2S,5S)-5-((R)-2-(2-hydroxyphenyl)-4,5-dihydrothiazol-4-yl)-1-methyl-N-(16- |
| | oxo-20-((3aS,4S,6aR)-2-oxohexahydro-1H-thieno[3,4-d]imidazol-4-yl)- |
| | 3,6,9,12-tetraoxa-15-azaicosyl)pyrrolidine-2-carboxamide |
| I-30G | (2S,5S)-5-((S)-2-(2-hydroxyphenyl)-4,5-dihydrothiazol-4-yl)-1-methyl-N-(16- |
| | oxo-20-((3aS,4S,6aR)-2-oxohexahydro-1H-thieno[3,4-d]imidazol-4-yl)- |
| | 3,6,9,12-tetraoxa-15-azaicosyl)pyrrolidine-2-carboxamide |
| I-31A | (2R,5R)-5-((R)-2-(2-hydroxyphenyl)-4,5-dihydrothiazol-4-yl)-1-methyl-N-(16- |
| | oxo-20-((3aS,4S,6aR)-2-oxohexahydro-1H-thieno[3,4-d]imidazol-4-yl)- |
| | 3,6,9,12-tetraoxa-15-azaicosyl)pyrrolidine-2-carboxamide |
| I-31B | (2R,5R)-5-((S)-2-(2-hydroxyphenyl)-4,5-dihydrothiazol-4-yl)-1-methyl-N-(16- |
| | oxo-20-((3aS,4S,6aR)-2-oxohexahydro-1H-thieno[3,4-d]imidazol-4-yl)- |
| | 3,6,9,12-tetraoxa-15-azaicosyl)pyrrolidine-2-carboxamide |
| I-31C | (2R,5S)-5-((S)-2-(2-hydroxyphenyl)-4,5-dihydrothiazol-4-yl)-1-methyl-N-(16- |
| | oxo-20-((3aS,4S,6aR)-2-oxohexahydro-1H-thieno[3,4-d]imidazol-4-yl)- |
| | 3,6,9,12-tetraoxa-15-azaicosyl)pyrrolidine-2-carboxamide |
| I-31D | (2S,5R)-5-((R)-2-(2-hydroxyphenyl)-4,5-dihydrothiazol-4-yl)-1-methyl-N-(16- |
| | oxo-20-((3aS,4S,6aR)-2-oxohexahydro-1H-thieno[3,4-d]imidazol-4-yl)- |
| | 3,6,9,12-tetraoxa-15-azaicosyl)pyrrolidine-2-carboxamide |
| I-31E | (2S,5R)-5-((S)-2-(2-hydroxyphenyl)-4,5-dihydrothiazol-4-yl)-1-methyl-N-(16- |
| | oxo-20-((3aS,4S,6aR)-2-oxohexahydro-1H-thieno[3,4-d]imidazol-4-yl)- |
| | 3,6,9,12-tetraoxa-15-azaicosyl)pyrrolidine-2-carboxamide |
| I-31F | (2S,5S)-5-((R)-2-(2-hydroxyphenyl)-4,5-dihydrothiazol-4-yl)-1-methyl-N-(16- |
| | oxo-20-((3aS,4S,6aR)-2-oxohexahydro-1H-thieno[3,4-d]imidazol-4-yl)- |
| | 3,6,9,12-tetraoxa-15-azaicosyl)pyrrolidine-2-carboxamide |
| I-31G | (2S,5S)-5-((S)-2-(2-hydroxyphenyl)-4,5-dihydrothiazol-4-yl)-1-methyl-N-(16- |
| | oxo-20-((3aS,4S,6aR)-2-oxohexahydro-1H-thieno[3,4-d]imidazol-4-yl)- |
| | 3,6,9,12-tetraoxa-15-azaicosyl)pyrrolidine-2-carboxamide |
| I-32A | 2-((R)-4-((2R,5S)-1-methyl-5-(prop-2-yn-1-ylcarbamoyl)pyrrolidin-2-yl)-4,5- |
| | dihydrothiazol-2-yl)phenyl sulfurofluoridate |
| I-32B | 2-((R)-4-((2S,5R)-1-methyl-5-(prop-2-yn-1-ylcarbamoyl)pyrrolidin-2-yl)-4,5- |
| | dihydrothiazol-2-yl)phenyl sulfurofluoridate |
| I-32C | 2-((R)-4-((2S,5S)-1-methyl-5-(prop-2-yn-1-ylcarbamoyl)pyrrolidin-2-yl)-4,5- |
| | dihydrothiazol-2-yl)phenyl sulfurofluoridate |
| I-32D | 2-((S)-4-((2R,5R)-1-methyl-5-(prop-2-yn-1-ylcarbamoyl)pyrrolidin-2-yl)-4,5- |
| | dihydrothiazol-2-yl)phenyl sulfurofluoridate |
| I-32E | 2-((S)-4-((2R,5S)-1-methyl-5-(prop-2-yn-1-ylcarbamoyl)pyrrolidin-2-yl)-4,5- |
| | dihydrothiazol-2-yl)phenyl sulfurofluoridate |
| I-32F | 2-((S)-4-((2S,5R)-1-methyl-5-(prop-2-yn-1-ylcarbamoyl)pyrrolidin-2-yl)-4,5- |
| | dihydrothiazol-2-yl)phenyl sulfurofluoridate |
| I-32G | 2-((S)-4-((2S,5S)-1-methyl-5-(prop-2-yn-1-ylcarbamoyl)pyrrolidin-2-yl)-4,5- |
| | dihydrothiazol-2-yl)phenyl sulfurofluoridate |
| I-33A | 2-((R)-4-((2R,5R)-1-methyl-5-(prop-2-yn-1-ylcarbamoyl)pyrrolidin-2-yl)-4,5- |
| | dihydrothiazol-2-yl)phenyl sulfurofluoridate |
| I-33B | 2-((R)-4-((2R,5S)-1-methyl-5-(prop-2-yn-1-ylcarbamoyl)pyrrolidin-2-yl)-4,5- |
| | dihydrothiazol-2-yl)phenyl sulfurofluoridate |
| I-33C | 2-((R)-4-((2S,5S)-1-methyl-5-(prop-2-yn-1-ylcarbamoyl)pyrrolidin-2-yl)-4,5- |
| | dihydrothiazol-2-yl)phenyl sulfurofluoridate |
| I-33D | 2-((S)-4-((2R,5R)-1-methyl-5-(prop-2-yn-1-ylcarbamoyl)pyrrolidin-2-yl)-4,5- |
| | dihydrothiazol-2-yl)phenyl sulfurofluoridate |
| I-33E | 2-((S)-4-((2R,5S)-1-methyl-5-(prop-2-yn-1-ylcarbamoyl)pyrrolidin-2-yl)-4,5- |
| | dihydrothiazol-2-yl)phenyl sulfurofluoridate |
| I-33F | 2-((S)-4-((2S,5R)-1-methyl-5-(prop-2-yn-1-ylcarbamoyl)pyrrolidin-2-yl)-4,5- |
| | dihydrothiazol-2-yl)phenyl sulfurofluoridate |
| I-33G | 2-((S)-4-((2S,5S)-1-methyl-5-(prop-2-yn-1-ylcarbamoyl)pyrrolidin-2-yl)-4,5- |
| | dihydrothiazol-2-yl)phenyl sulfurofluoridate |

or a pharmaceutically acceptable salt thereof.

### Pharmaceutical Compositions

The disclosure provides pharmaceutical compositions containing the compounds disclosed herein. In some embodiments, the pharmaceutical compositions contain (2S,5S)-5-((S)-2-(2-hydroxyphenyl)-4,5-dihydrothiazol-4-yl)-1-methylpyrrolidine-2-carboxylic acid. In other embodiments, the pharmaceutical compositions contain (2S,5S)-5-((S)-2-(2-hydroxyphenyl)-4,5-dihydrooxazol-4-yl)-1-methylpyrrolidine-2-carboxylic acid.

The term “pharmaceutically acceptable salt,” as used herein, includes salts that are suitable for administration to a subject as defined herein. Pharmaceutically acceptable salts are well known in the art. See, e.g., Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The compounds described herein may be synthetically prepared whether or not found in nature. When found in nature, such compounds may also be isolated.

In addition to the compounds disclosed herein, the pharmaceutical compositions may contain one or more pharmaceutically acceptable excipients. The pharmaceutically acceptable is selected on the basis of the mode and route of administration. Suitable pharmaceutical carriers, as well as pharmaceutical necessities for use in pharmaceutical formulations, are described in Remington: The Science and Practice of Pharmacy, 21^{st} Ed., Gennaro, Ed., Lippencott Williams & Wilkins (2005); Handbook of Pharmaceutical Excipients, 6^{th} Edition, Rowe et al., Eds., Pharmaceutical Press (2009); and the USP/NF (United States Pharmacopeia and the National Formulary).

In some embodiments, the pharmaceutically acceptable excipient is one or more of an antioxidant, binder, buffer, coloring agent, diluent (e.g., solid or liquid), disintegrant, dispersing agent, dyestuff, filler, emulsifier, flavoring agent, lubricant, pH adjuster, pigment, preservative, stabilizer, solubilizing agent, solvent, suspending agent, sweetener, or wetting agent, or combination thereof.

Examples of suitable excipients include, without limitation, acacia, alginate, calcium phosphate, calcium carbonate, calcium silicate, carbopol gel, carboxymethyl cellulose, carnauba wax, cellulose, crospovidone, dextrose, diacetylated monoglycerides, ethylcellulose, gelatin, glyceryl monostearate 40-50, gum acacia, gum arabic, hydroxyethyl cellulose, hydroxypropyl cellulose, hydroxypropylmethyl cellulose, hypromellose phthalate, hypromellose, lactose, lecithin, magnesium stearate, kaolin, methacrylic acid copolymer type C, mannitol, methyl cellulose, methylhydroxybenzoate, microcrystalline cellulose, povidone, polyethylene glycol, polysorbate 80, polyvinylpyrrolidone, propylhydroxybenzoate, sodium carboxymethyl cellulose sodium hydroxide, sodium stearyl fumarate, sodium starch glycolate, starch, sorbitan monooleate sorbitol, sorbic acid, sucrose, talc, tragacanth, talc, triethyl citrate, titanium dioxide, yellow ferric oxide, talc, oil medium (e.g., peanut oil, liquid paraffin, mineral oil, olive oil, almond oil, glycerin, propylene glycol), or water,

When the excipient serves as a diluent, it can be a solid, semisolid, or liquid material (e.g., normal saline), which acts as a vehicle, carrier or medium for the active ingredient. As is known in the art, the type of diluent can vary depending upon the intended route of administration.

The pharmaceutical compositions can be manufactured in a conventional manner, e.g., by conventional mixing, dissolving, granulating, dragee-making, levigating, emulsifying, encapsulating, entrapping, or lyophilizing processes. Methods well known in the art for making formulations are known in the art. See, e.g., Remington: The Science and Practice of Pharmacy, 21^{st} Ed., Gennaro, Ed., Lippencott Williams & Wilkins (2005), and Encyclopedia of Pharmaceutical Technology, eds. J. Swarbrick and J. C. Boylan, 1988-1999, Marcel Dekker, New York.

### Dosage Forms

The disclosure also provides dosage forms containing one or more of the compounds disclosed herein or the pharmaceutical compositions disclosed herein. One skilled in the art would be able to select a dosage form for use herein. For example, the dosage form may be a solid dosage form, liquid dosage form, or solid/liquid dosage form. In certain aspects, the dosage form is a solid dosage form. In other aspects, the dosage form is a liquid dosage form.

The dosage form may be formulated for the delivery that is most useful to the subject. In some embodiments, the dosage form is for enteral or parenteral administration. Examples of enteral administration include, without limitation, oral, rectal, sublingual, or buccal.

In other embodiments the dosage form is for parenteral administration, i.e., a parenteral dosage form. As used herein, the term “parenteral” refers to routes of administration aside from enteral administration. Examples of parenteral administration include, without limitation, buccal, epicutaneous, epidural, extra-amniotic, intra-arterial, intra-articular, intracardiac, intracavernous, intracerebral, intracerebroventricular, intradermal, intralesional, intramuscular, intraocular, intraosseous infusion, intraperitoneal, intrapulmonary, intrathecal, intrauterine, intravaginal, intravenous, intravesical, intravitreal, nasal, perivascular, subcutaneous, sublingual, transdermal, topical, transepithelial, or transmucosal. Parenteral administration may be by continuous infusion over a selected period of time. In certain embodiments, the dosage form is administered intravenously, intraperitoneally, intramuscularly, or subcutaneously. In further embodiments, the dosage form is administered orally, intravenously, intraperitoneally, intramuscularly, intradermally, or subcutaneously. In further embodiments, the dosage form is administered orally. In yet other embodiments, the dosage form is administered intradermally, intramuscularly, or subcutaneously.

Parenteral dosage forms are known in the art and include, without limitation, injectable solutions, inhalants, infusions, patches, depots, and suppositories. In certain aspects, the parenteral dosage form is an injectable solution. In other embodiments, the dosage form is formulated for oral delivery, i.e., an oral dosage form. In certain aspects, the oral dosage form is a pill (e.g., tablet, caplet, capsule (e.g., soft gelatin, hard gelatin, gel capsule)), effervescent dosage form, elixir, film, liquid/solution (e.g., suspension, emulsion), lollipop, lozenge, paste, powder, sachet, or syrup. In further aspects, the oral dosage form is a pill, tablet, capsule, syrup, liquid solution, powder, paste, patch, pump, or film. In yet other aspects, the oral dosage form is a dry product for reconstitution with water or other suitable vehicle before use.

When the dosage form is a solid dosage form, an enteric coating can be applied or the solid dosage form may be scored. An enteric coating can be stable at low pH (e.g., in the stomach) and can dissolve at higher pH (e.g., in the small intestine).

Regardless of the type of dosage form, it contains a therapeutically effective amount of the compound disclosed herein. One of skill in the art can determine a suitable amount of the compound disclosed herein to incorporate into the pharmaceutical compositions or dosage forms of the disclosure. In certain embodiments, the pharmaceutical composition or dosage form contains about 0.01 to about 1000 mg of one or more of the compounds disclosed herein. In other embodiments, the pharmaceutical composition or dosage form contains about 0.01, about 0.1, about 0.5, about 1, about 5, about 10, about 25, about 50, about 75, about 100, about 125, about 150, about 175, about 200, about 225, 250, about 275, about 300, about 350, about 400, about 450, about 500, about 550, about 600, about 650, about 700, about 750, about 800, about 850, about 900, about 950, or about 1000 mg of one or more of the compounds disclosed herein. In further embodiments, the pharmaceutical composition or dosage form contains about 0.01 to about 750, about 0.01 to about 500, about 0.01 to about 250, about 0.01 to about 100, about 0.01 to about 50, about 0.01 to about 25, about 0.01 to about 10, about 0.01 to about 5, about 0.01 to about 0.1, about 0.1 to about 1000, about 0.1 to about 750, about 0.1 to about 500, about 0.1 to about 250, about 0.1 to about 100, about 0.1 to about 50, about 0.1 to about 25, about 0.1 to about 10, about 0.1 to about 5, about 0.1 to about 1, about 1 to about 1000, about 1 to about 750, about 1 to about 500, about 1 to about 250, about 1 to about 100, about 1 to about 50, about 1 to about 25, about 1 to about 10, about 1 to about 5, about 5 to about 1000, about 5 to about 750, about 5 to about 500, about 5 to about 250, about 5 to about 100, about 5 to about 50, about 5 to about 25, about 5 to about 10, about 10 to about 1000, about 10 to about 750, about 10 to about 500, about 10 to about 250, about 10 to about 100, about 10 to about 50, about 10 to about 25, about 25 to about 1000, about 25 to about 750, about 25 to about 500, about 25 to about 250, about 25 to about 100, about 25 to about 50, about 50 to about 1000, about 50 to about 750, about 50 to about 500, about 50 to about 250, about 50 to about 100, about 100 to about 1000, about 100 to about 750, about 100 to about 500, about 100 to about 250, about 250 to about 1000, about 250 to about 750, about 250 to about 500, about 500 to about 1000, about 500 to about 750, or about 750 to about 1000 mg of one or more of the compounds disclosed herein.

### Methods of Use

The compounds of the disclosure are useful in modulating an inflammatory mediator, e.g., modulating an inflammation response (e.g., in a subject). In some aspects, the compounds can be used in methods of treating inflammation in a subject in need thereof. The methods include administering a compound, pharmaceutical composition or dosage form disclosed herein to the subject in need thereof.

“Treating” or variations thereof refers ameliorating or reducing the development of a disease or disorder, i.e., delaying the onset of the disease. In certain embodiments, “treating” refers to ameliorating or reducing at least one physical parameter of the disease or disorder. In other embodiments, “treating” is directed to improving the disease or disorder. In further embodiments, “treating” is directed to the cause of the disease or disorder. In yet other embodiments, “treating” is directed to relieving symptoms of the disease or disorder. In still further embodiments, “treating” is directed to treating the disease or disorder as a supplement another therapy.

The term “inflammation” as used herein refers to a physical condition in which a part of the subject's body becomes inflamed. Signs of inflammation include, e.g., redness, welling, heat, pain, and/or loss of function. One skilled in the art, e.g., an attending physician, would be able to recognize such signs. In some embodiments, the inflammation is localized, i.e., restricted to one particular area of the subject's body. In other embodiments, the inflammation is systemic, i.e., in two or more areas of the subject's body, including the subject's whole body.

The disclosure further provides methods of decreasing inflammation in a subject in need thereof. The methods include administering the compounds, pharmaceutical compositions or dosage forms described herein to the subject in need thereof. In doing so, one or more of the signs of inflammation are decreased. In some embodiments, the methods result in decreasing redness. In other embodiments, the methods result in decreasing swelling. In further embodiments, the methods result in decreasing heat. In yet other embodiments, the methods result in decreasing temperature/heat. In still further embodiments, the methods result in decreasing pain. In other embodiments, the methos results in increasing function.

The disclosure also provides methods of decreasing an inflammatory marker in a subject in need thereof. The methods include administering the compounds, pharmaceutical compositions or dosage forms disclosed herein to the subject in need thereof. The term “inflammatory marker” refers to an internal indication or sign of inflammation in a subject. One skilled in the art would be able to select the particular inflammatory marker to monitor or evaluate. In some embodiment, the inflammatory marker is one or more of a complete blood count (CBC), C reactive protein (CRP), erythrocyte sedimentation rate (ESR), plasma viscosity (PV), fibrinogen, ferritin, procalcitonin, or calprotectin, among others. In some embodiments, the inflammatory marker is a CBC. In other embodiments, the inflammatory marker is CRP. In further embodiments, the inflammatory marker is ESR. In yet other embodiments, the inflammatory marker is PV. In still further embodiments, the inflammatory marker is fibrinogen. In other embodiments, the inflammatory marker is ferritin. In further embodiments, the inflammatory marker is procalcitonin. In still other embodiments, the inflammatory marker is calprotectin. One of skill in the art would be able to determine how to measure any one of these markers and what would constitute elevated levels for each marker.

The methods herein also permit modulating levels of one or more inflammatory regulatory molecules or inflammatory mediators in a subject in need thereof. One of skill in the art would be able to determine the inflammatory regulatory molecules or mediator to modulate. For example, recent studies have shown that the interleukin 1 (IL-1)-interleukin 1 receptor antagonist (IL-1ra) axis regulates vaccine-mediated systemic inflammation in a host-specific manner. In human immune cells, RNA vaccines induce production of IL-1 cytokines, predominantly IL-1β, which is dependent on both the RNA and lipid formulation. IL-1 in turn triggers the induction of the broad spectrum of pro-inflammatory cytokines (including IL-6).

In certain examples, the inflammatory regulatory molecule is a cytokine. In some embodiments, the cytokine is an interleukin (IL), tumor necrosis factor alpha (TNF-α), interferon gamma (IFNγ), or granulocyte-macrophage colony stimulating factor (GM-CSF). In some embodiments, the cytokine is TNF-α. In other embodiments, the cytokine is IFNγ. In further embodiments, the cytokine is GM-CSF. In yet other embodiments, the cytokine is an interleukin. Examples of interleukins include, without limitation, IL-1 (including IL-1a and IL-1β), IL-4, IL-6, IL-8, IL-9, IL-10, IL-11, IL-12, IL-13, IL-17, IL-19, IL-23, IL-35, and IL-36. In other examples, the inflammatory mediator is a prostaglandin or chemokine. In some embodiments, the inflammatory mediator is a chemokine such as GRO-α. In other embodiments, the inflammatory mediator is a prostaglandin such as prostaglandin E₂ (PGE2).

The compounds disclosed herein may be used to treat inflammatory bowel disease. The term “inflammatory bowel disease” or “IBD” as used herein refers to a disorder that involves chronic inflammation of the digestive tract. IBD includes ulcerative colitis and Crohn's disease. In some embodiments, the IBD is ulcerative colitis. In other embodiments, the IBD is Crohn's disease. In further embodiments, the IBD includes ulcers in the intestinal lining. In yet other embodiments, the IBD includes inflammation of the intestinal lining. The subject may have inflammatory bowel disease or may at risk of developing inflammatory bowel disease. In some embodiments, the patient has or has been diagnosed with inflammatory bowel disease. In other embodiments, the patient is a risk of developing inflammatory bowel disease.

The compounds also may be used to treat sepsis.

The compounds or pharmaceutical compositions may be administered as described herein in combination with another pharmaceutical agent. In some embodiments, the another pharmaceutical agent is an anti-inflammatory medication. In some embodiments, the disclosed compound or pharmaceutical composition is administered before the anti-inflammatory medication. In other embodiments, the disclosed compound or pharmaceutical composition is administered after the anti-inflammatory medication. In further embodiments, the disclosed compound or pharmaceutical composition is administered concurrently with the anti-inflammatory medication.

The anti-inflammatory medication may be selected by one of skill in the art depending on the subject. In some embodiments, the anti-inflammatory medication is a steroid. Examples of steroid useful for treating inflammation include, without limitation, corticosteroids such as cortisone, prednisone or methylprednisolone. In other embodiments, the anti-inflammatory medication is a nonsteroidal anti-inflammatory drug (NSAID). Examples of NSAIDs include, without limitation, diclofenac, diflunisal, etodolac, fenoprofen, flurbiprofen, indomethacin, ibuprofen, ketoprofen, ketorolac, mefenamic acid, meloxicam, nabumetone, naproxen, oxaprozin, piroxicam, sulindac, or tolmetin, or combination thereof.

The disclosure also provides methods of monitoring the compounds of formula I, or metabolites thereof, using techniques known in the art. For example, the compounds, or metabolites thereof, may be traced in vivo after administration. In doing so, the compounds are labelled with an appropriate isotope. Examples of isotopes include deuterium (²H, D), tritium (³H), ¹¹C, ¹³C, ¹⁴C, or ¹⁸F as described herein. Suitable techniques for monitoring the isotopically labeled compounds include, without limitation, mass spectrometry, autoradiography, PET imaging, or combinations thereof.

In addition, deuterium (²H, D) may be used in place of hydrogen (¹H, H) anywhere on the compounds of formula I using techniques known in the art in order to potentially improve pharmaceutical properties, e.g., by reducing metabolism or by shifting the ratio of metabolites formed to favor more prolonged efficacy, greater efficacy, less toxicity or any combination of these outcomes.

### Probes

In a further embodiment, the compounds disclosed herein may be used as probes. The probes may be used as determined by those skilled in the art. For example, the probes may be used in diagnostic methods. For example, the probes may be used to identify proteins which bind to one or more compounds (e.g., active compounds) described herein. Binding of a molecule to a protein may in some cases modulate that protein's activity in ways that affect a mammal's health status. Knowledge of the functional modulation of one or more identified proteins may be helpful in patient selection, biomarker selection, and other aspects related to translation to clinical practice.

In some embodiments, the probe contains a compound comprising a diazirinyl, alkynyl, biotinyl, or fluorosulfate-containing moiety or a combination thereof. The term “fluorosulfate-containing moiety” as used herein refers to a chemical moiety that comprises a OSO₂F group. In certain aspects, the fluorosulfate-containing moiety is an -alkyl-SO₃F. In other aspects, the fluorosulfate-containing moiety is OSO₂F. In other embodiments, the probe contains a compound discussed herein comprising an diazirinyl. In further embodiments, the probe contains a compound discussed herein comprising an alkynyl. In yet other embodiments, the probe contains a compound discussed herein comprising a biotinyl. In still further embodiments, the probe contains a compound discussed herein comprising a diazirinyl comprising an alkynyl substituent. In other embodiments, the probe contains a compound discussed herein comprising C(O)-alkynyl. In further embodiments, the probe is compound J, K, or L. In still other embodiments, the probe is compound I-13, I-14, I-15, I-16, I-17, or I-18. In yet further embodiments, the probe is compound I-28A, I-28B, I-28C, I-28D, I-28E, I-28F, I-28G, I-29A, I-29B, I-29C, I-29D, I-29E, I-29F, I-29G, I-30A, I-30B, I-30C, I-30D, I-30E, I-30F, I-30G, I-31A, I-31B, I-31C, I-31D, I-31E, I-31F, I-31G, I-32A, I-32B, I-32C, I-32D, I-32E, I-32F, I-32G, I-33A, I-33B, I-33C, I-33D, I-33E, I-33F, or I-33G

### Aspects

Aspect 1. A compound of Formula (I):

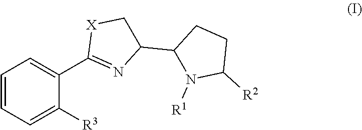

wherein:
R¹ is C₁₋₆alkyl or C₂₋₆alkenyl;
R² is H, C(O)OH, or a masked carboxylic acid;
R³ is H or OH;
X is O or S; and
   or a pharmaceutically acceptable salt thereof;
   with the proviso that the compound is not (2S,5S)-5-((S)-2-(2-hydroxyphenyl)-4,5-dihydrothiazol-4-yl)-1-methylpyrrolidine-2-carboxylic acid or (2S,5S)-5-((S)-2-(2-hydroxyphenyl)-4,5-dihydrooxazol-4-yl)-1-methylpyrrolidine-2-carboxylic acid.

Aspect 2. The compound of Aspect 1, wherein R¹ is C₁alkyl, C₂alkyl, C₃alkyl, C₄alkyl, C₅alkyl, or C₆alkyl, such as CH₃.

Aspect 3. The compound of Aspect 1, wherein R¹ is C₂alkenyl, C₃alkenyl, C₄alkenyl, C₅alkenyl, or C₆alkenyl.

Aspect 4. The compound of any one of the preceding Aspects, wherein R² is C(O)OH.

Aspect 5. The compound of any one of Aspects 1-4, wherein R² is a masked carboxylic acid, such as —C(O)OC₁₋₆alkyl, —C(O)SC₁₋₆alkyl, oxazolyl, CN, —C(O)NH₂, —C(O)NHR^{A}, —C(O)N(R^{A})₂, —C(O)OC₁₋₆alkylOH, or —C(O)Ophenyl, wherein each R^{A} is, independently, C₁₋₆alkyl, cycloalkyl, aryl, —CH₂OH, phenyl, —CH₂OC₁₋₆alkyl, or —C(OC₁₋₆alkyl)₃.

Aspect 6. The compound of any one of Aspects 1-4, wherein R² is H.

Aspect 7. The compound of any one of the preceding Aspects, wherein R³ is H.

Aspect 8. The compound of any one of Aspects 1-7, wherein R³ is OH.

Aspect 9. The compound of any one of the preceding Aspects, wherein X is O.

Aspect 10. The compound of any one of Aspects 1-9, wherein X is S.

Aspect 11. The compound of Aspect 1, that is of formula (II):

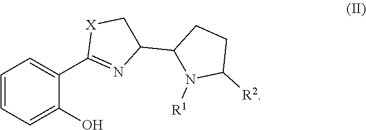

Aspect 12. The compound of Aspect 1, that is of formula (III):

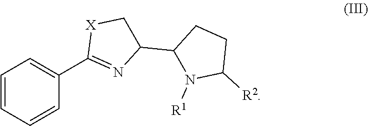

Aspect 13. The compound of Aspect 1, that is of formula (IV):

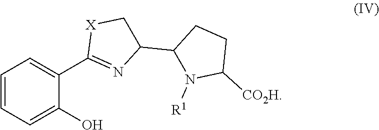

Aspect 14. The compound of Aspect 1, that is of formula (V):

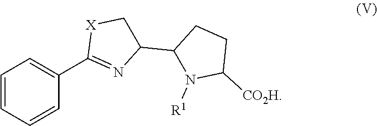

Aspect 15. The compound of Aspect 1, that is:

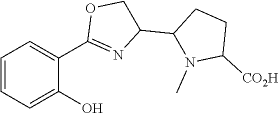 [SMILES: CN1C(C(=O)O)CCC1C1COC(c2ccccc2O)=N1]

(5-(2-(2-hydroxyphenyl)-4,5-dihydrooxazol-4-yl)-1-methylpyrrolidine-2-carboxylic acid), such as:

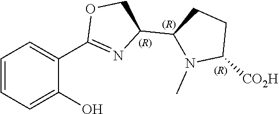

((2R,5R)-5-((R)-2-(2-hydroxyphenyl)-4,5-dihydrooxazol-4-yl)-1-methylpyrrolidine-2-carboxylic acid); or

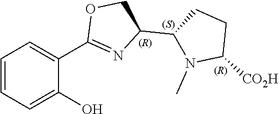

((2R,5S)-5-((R)-2-(2-hydroxyphenyl)-4,5-dihydrooxazol-4-yl)-1-methylpyrrolidine-2-carboxylic acid).

Aspect 16. The compound of Aspect 1, that is:

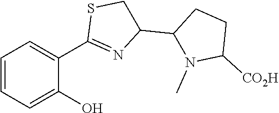 [SMILES: CN1C(C(=O)O)CCC1C1CSC(c2ccccc2O)=N1]

(5-(2-(2-hydroxyphenyl)-4,5-dihydrothiazol-4-yl)-1-methylpyrrolidine-2-carboxylic acid), such as:

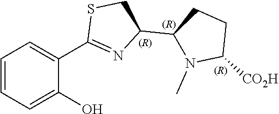

((2R,5S)-5-((R)-2-(2-hydroxyphenyl)-4,5-dihydrothiazol-4-yl)-1-methylpyrrolidine-2-carboxylic acid); or

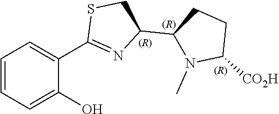

((2R,5R)-5-((R)-2-(2-hydroxyphenyl)-4,5-dihydrothiazol-4-yl)-1-methylpyrrolidine-2-carboxylic acid).

Aspect 17. A pharmaceutical composition comprising a pharmaceutically acceptable excipient and the compound a compound of Formula (I):

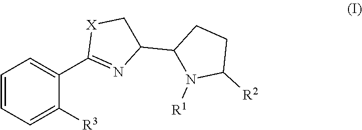

wherein:
R¹ is C₁₋₆alkyl or C₂₋₆alkenyl;
R² is H, C(O)OH or a masked carboxylic acid;
R³ is H or OH;
X is O or S; and
   or a pharmaceutically acceptable salt thereof.

Aspect 18. The pharmaceutical composition of Aspect 18, wherein R¹ is C₁alkyl, C₂alkyl, C₃alkyl, C₄alkyl, C₅alkyl, or C₆alkyl, such as CH₃.

Aspect 19. The pharmaceutical composition of Aspect 18, wherein R¹ is C₂alkenyl, C₃alkenyl, C₄alkenyl, C₅alkenyl, or C₆alkenyl.

Aspect 20. The pharmaceutical composition of Aspect 18, wherein R¹ is H.

Aspect 21. The pharmaceutical composition of any one of Aspects 18-21, wherein R² is C(O)OH.

Aspect 22. The pharmaceutical composition of any one of Aspects 81-21, wherein R² is a masked carboxylic acid, such as —C(O)OC₁₋₆alkyl, —C(O)SC₁-6alkyl, oxazolyl, CN, —C(O)NH₂, —C(O)NHR^{A}, —C(O)N(R^{A})₂, —C(O)OC₁₋₆alkylOH, or —C(O)Ophenyl, wherein each R^{A} is, independently, C₁₋₆alkyl, cycloalkyl, aryl, —CH₂OH, phenyl, —CH₂OC₁₋₆alkyl, or —C(OC₁₋₆alkyl)₃.

Aspect 23. The pharmaceutical composition of any one of Aspects 18-21, wherein R² is H.

Aspect 24. The pharmaceutical composition of any one of Aspects 18-24, wherein R³ is H.

Aspect 25. The pharmaceutical composition of any one of Aspects 18-24, wherein R³ is OH.

Aspect 26. The pharmaceutical composition of any one of Aspects 18-26, wherein X is O.

Aspect 27. The pharmaceutical composition of any one of Aspects 18-26, wherein X is S.

Aspect 28. The pharmaceutical composition of Aspect 1, wherein the compound is of formula (II):

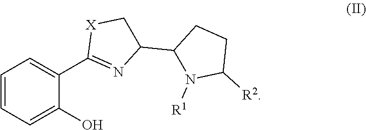

Aspect 29. The pharmaceutical composition of Aspect 1, wherein the compound is of formula (III):

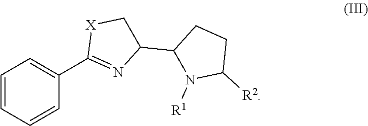

Aspect 30. The pharmaceutical composition of Aspect 1, wherein the compound is of formula (IV):

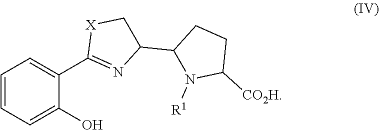

Aspect 31. The pharmaceutical composition of Aspect 1, wherein the compound is of formula (V):

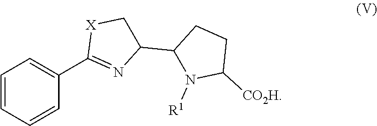

Aspect 32. The pharmaceutical composition of Aspect 16, wherein the compound is:

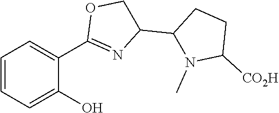 [SMILES: CN1C(C(=O)O)CCC1C1COC(c2ccccc2O)=N1]

(5-(2-(2-hydroxyphenyl)-4,5-dihydrooxazol-4-yl)-1-methylpyrrolidine-2-carboxylic acid), such as:

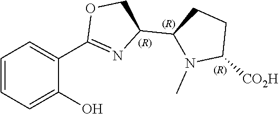

((2R,5R)-5-((R)-2-(2-hydroxyphenyl)-4,5-dihydrooxazol-4-yl)-1-methylpyrrolidine-2-carboxylic acid); or

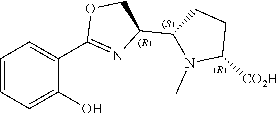

((2R,5S)-5-((R)-2-(2-hydroxyphenyl)-4,5-dihydrooxazol-4-yl)-1-methylpyrrolidine-2-carboxylic acid).

Aspect 33. The pharmaceutical composition of Aspect 16, wherein the compound is:

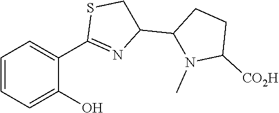 [SMILES: CN1C(C(=O)O)CCC1C1CSC(c2ccccc2O)=N1]

(5-(2-(2-hydroxyphenyl)-4,5-dihydrothiazol-4-yl)-1-methylpyrrolidine-2-carboxylic acid), such as:

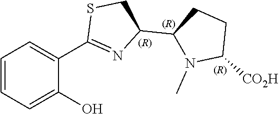

((2R,5S)-5-((R)-2-(2-hydroxyphenyl)-4,5-dihydrothiazol-4-yl)-1-methylpyrrolidine-2-carboxylic acid); or

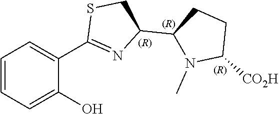

((2R,5R)-5-((R)-2-(2-hydroxyphenyl)-4,5-dihydrothiazol-4-yl)-1-methylpyrrolidine-2-carboxylic acid).

Aspect 34. A dosage form comprising the compound of any one of Aspects 1-17 or the pharmaceutical composition of any one of Aspects 18-34.

Aspect 35. The dosage form of Aspect 35 that is formulated for oral, intravenous, intraperitoneal, intramuscular, intradermal or subcutaneous administration.

Aspect 36. The dosage form of Aspect 35 that is formulated for intradermal, intramuscular, or subcutaneous administration.

Aspect 37. A method of treating inflammation in a subject in need thereof, comprising administering the compound of any one of Aspects 1-17, pharmaceutical composition of any one of Aspects 18-34 or the dosage form of any one of Aspects 35-37 to the subject in need thereof.

Aspect 38. A method of decreasing inflammation in a subject in need thereof, comprising administering the compound of any one of Aspects 1-17, pharmaceutical composition of any one of Aspects 18-34 or the dosage form of any one of Aspects 35-37 to the subject in need thereof.

Aspect 39. A method of decreasing an inflammatory marker in a subject in need thereof, comprising administering the compound of any one of Aspects 1-17, pharmaceutical composition of any one of Aspects 18-34 or the dosage form of any one of Aspects 35-37 to the subject in need thereof.

The following examples, while illustrative individual embodiments, are not intended to limit the scope of the described invention, and the reader should not interpret them in this way.

### EXAMPLES

### Example 1. (2R,5R)-5-((R)-2-(2-hydroxyphenyl)-4,5-dihydrooxazol-4-yl)-1-methylpyrrolidine-2-carboxylic acid

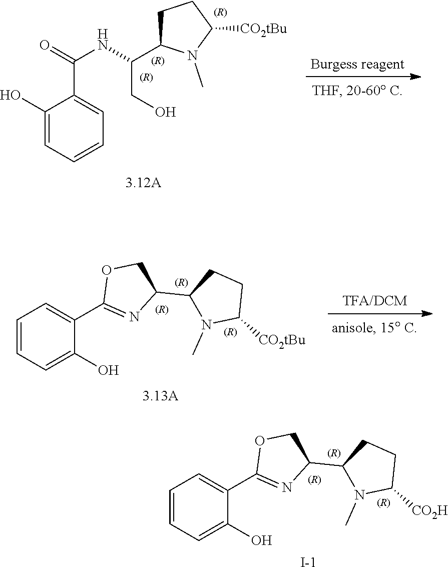

Synthesis of 3.13A. To a solution of tert-butyl(2R,5R)-5-[(1R)-2-hydroxy-1-[(2-hydroxybenzoyl)amino]ethyl]-1-methyl-pyrrolidine-2-carboxylate (3.12A, 100.00 mg, 274.40 μmol, 1 eq) in THF (2 mL) was added Burgess reagent (98.09 mg, 411.60 μmol, 1.5 eq) at 20° C. and the mixture was stirred at 60° C. for 1 hr. The reaction mixture was concentrated under reduced pressure to give a residue. The residue was purified by flash silica gel chromatography (ISCO®; 5 g SepaFlash® Silica Flash Column, Eluent of 0~10% Ethyl acetate/Petroleum ether gradient @ 45 mL/min) to give tert-butyl(2R,5R)-5-((R)-2-(2-hydroxyphenyl)-4,5-dihydrooxazol-4-yl)-1-methylpyrrolidine-2-carboxylate (3.13A, 58 mg, 61.02% yield) as a colorless oil. (ES, m/z): [M+1]+347.2.

Synthesis of I-1. To a solution of tert-butyl(2R,5R)-5-[(4R)-2-(2-hydroxyphenyl)-4,5-dihydrooxazol-4-yl]-1-methyl-pyrrolidine-2-carboxylate (3.13A, 58 mg, 167.43 μmol, 1 eq) in DCM (0.1 mL) was added anisole (181.05 mg, 1.67 mmol, 181.96 μL, 10 eq) and TFA (0.9 mL) at 15° C. and the mixture was stirred at 15° C. for 16 hrs. The reaction mixture was concentrated under reduced pressure to give a residue. The crude product was purified by prep-HPLC (column: Waters Xbridge Prep OBD C18 150*40 mm*10 μm; mobile phase: [H₂O (10 mM NH₄HCO₃)-ACN]; gradient: 10%-45% B over 8.0 min) to give 10.78 mg of I-1 (22%) as a pink solid. (ES, m/z): [M+1]+291.0.

I-1: ¹H NMR (400 MHz, chloroform-d) d=7.66 (br d, J=7.4 Hz, 1H), 7.42 (br t, J=7.4 Hz, 1H), 7.02 (d, J=8.4 Hz, 1H), 6.90 (t, J=7.6 Hz, 1H), 4.86 (br s, 1H), 4.59 (br t, J=9.2 Hz, 1H), 4.17 (br t, J=8.0 Hz, 1H), 3.95-3.78 (m, 2H), 2.89 (br s, 3H), 2.34 (br d, J=2.0 Hz, 1H), 2.22-2.11 (m, 1H), 2.11-1.96 (m, 1H), 1.84 (br s, 1H).

### Example 2. (2R,5S)-5-((R)-2-(2-hydroxyphenyl)-4,5-dihydrooxazol-4-yl)-1-methylpyrrolidine-2-carboxylic acid

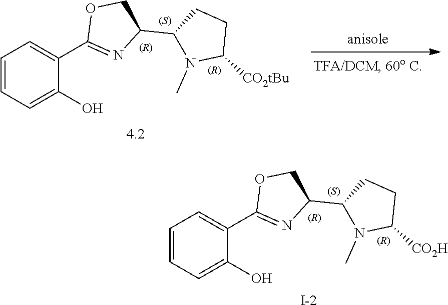

Synthesis of I-2. A mixture of tert-butyl(2R,5S)-5-[(4R)-2-(2-hydroxyphenyl)-4,5-dihydrooxazol-4-yl]-1-methyl-pyrrolidine-2-carboxylate (4.2, 200 mg, 577.33 μmol, 1 eq), anisole (624.32 mg, 5.77 mmol, 627.46 μL, 10 eq) in TFA (6.75 mL) and DCM (0.75 mL) was degassed and purged with N₂ for 3 times, and then the mixture was stirred at 60° C. for 6 hrs under N₂ atmosphere. The residue was purified by prep-HPLC (neutral condition; column: Phenomenex Gemini-NX 80*40 mm*3 μm; mobile phase: [H₂O (10 mM NH₄HCO₃)-ACN]; gradient: 5%-25% B over 8.0 min) to give 12.29 mg of I-2 (7.3%) as a white solid. (ES, m/z): [M+1]+291.1.

I-2: ¹H NMR (400 MHz, chloroform-d) 8=7.64-7.66 (d, J=8.0 Hz, 1H), 7.38-7.41 (t, J=4.0 Hz, 1H), 7.01-7.03 (d, J=8.0 Hz, 1H), 6.88-6.91 (t, J=8.0 Hz, 1H), 4.54-4.59 (m, 1H), 4.49-4.52 (m, 1H), 4.18-4.21 (t, J=8.0 Hz, 1H), 3.46-3.47 (d, J=4.0 Hz, 1H), 3.05-3.09 (m, 1H), 2.76 (s, 3H), 2.19-2.25 (m, 2H), 1.98-2.01 (m, 1H), 1.59-1.64 (m, 1H).

### Example 3. (2R,5R)-5-((R)-2-(2-hydroxyphenyl)-4,5-dihydrothiazol-4-yl)-1-methylpyrrolidine-2-carboxylic acid

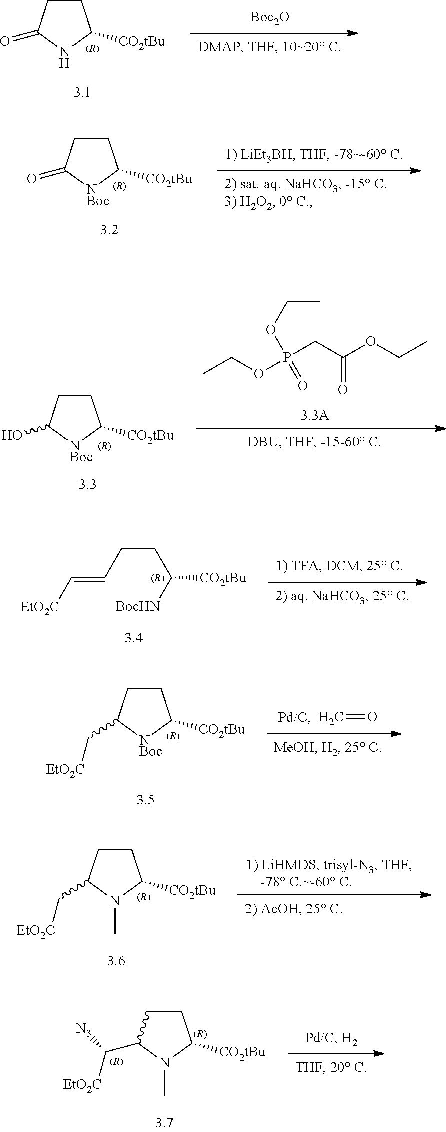

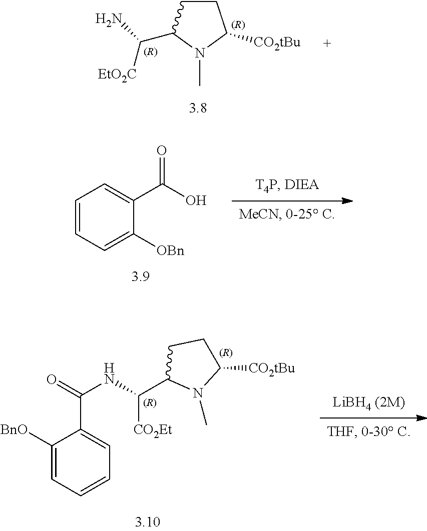

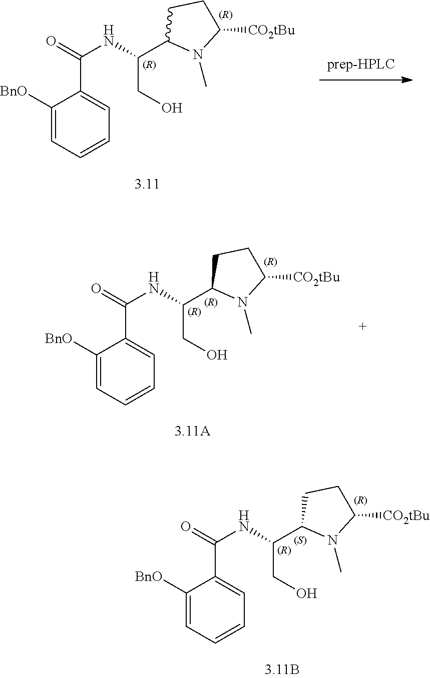

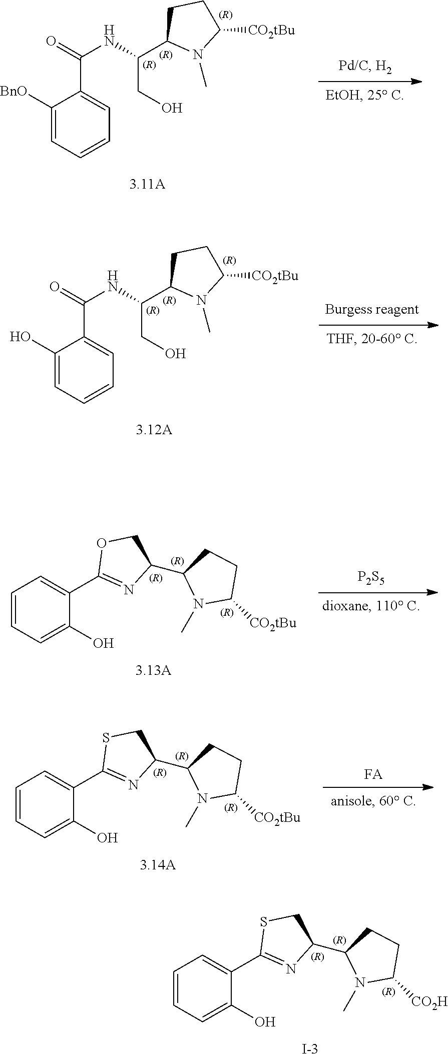

Synthesis of 3.2. The reactions were set up in 10 batches in parallel. To a solution of tert-butyl(2R)-5-oxopyrrolidine-2-carboxylate (3.1, 200 g, 1.08 mol, 1 eq) in THF (1.4 L) was added DMAP (26.38 g, 215.96 mmol, 0.2 eq) at 20° C. and then Boc₂O (282.80 g, 1.30 mol, 297.68 mL, 1.2 eq) was added dropwise to the solution at 10° C. The mixture was stirred at 20° C. for 16 hrs. Then the batches were combined for work-up. The reaction mixture was concentrated under reduced pressure to give a residue. The residue was purified by column chromatography (SiO₂, Petroleum ether/Ethyl acetate=1/0 to 5/1) to give 3.15 kg of 3.2 (crude) as an off-white solid.

Synthesis of 3.3. The reactions were set up in 2 batches in parallel. Charge THF (8750 mL) to reactor at 20° C.~25° C. Charge di-tert-butyl (R)-5-oxopyrrolidine-1,2-dicarboxylate (3.2, 1750 g, 6.13 mol, 1 eq) to reactor at 20° C. The reaction was cooled down to −60° C.~−78° C. under N₂. LiEt₃BH (1 M, 6.19 L, 1.01 eq) was added dropwise to the solution using peristaltic pump (100 mL/min) under N₂. The reaction was stirred at −60° C.~-78° C. for 30 min under N₂. The reaction mixture warm to −15° C. NaHCO₃(aq) (7 L) was dropwise added to the solution using peristaltic pump (150 mL/min) under N₂. The reaction was stirred at −15° C. for 30 min under N₂. H₂O₂ (4.13 kg, 36.43 mol, 3.50 L, 30% purity, 5.94 eq) was added dropwise to the solution using peristaltic pump (100 mL/min) under N₂. The reaction was stirred at 0° C. for 30 min under N₂. Two batches were combined for work-up. The solution was decanted from a white solid residue and extracted with Ethyl acetate (4 L×2). Washed with sat. aq. NaHCO₃ (4 L) and brine (4 L), dried over Na₂SO₄, filtered and evaporated under reduced pressure to give 3.1 kg of 3.3 (crude) as a yellow oil, which was used to next step directly without purification.

Synthesis of 3.4. The reactions were set up in 2 batches in parallel. Charge THF (12.32 L) to reactor at 20~25° C. Charged ethyl 2-diethoxyphosphorylacetate (3.3A, 1.56 kg, 6.96 mol, 1.38 L, 1.3 eq) to reactor at 20~25° C. DBU (1.22 kg, 8.03 mol, 1.21 L, 1.5 eq) was dropwise added to the solution using peristaltic pump (100 mL/min) under N₂. The mixture was stirred at 20~25° C. for 2 hrs under N₂. The reaction was cooled down to −15° C.~-10° C. under N₂. A solution of di-tert-butyl(2R)-5-hydroxypyrrolidine-1,2-dicarboxylate (3.3, 1.54 kg, 5.36 mol, 1 eq) in THF (3.08 L) was dropwise added to the solution using peristaltic pump (250 mL/min) under N₂. The mixture was stirred at 60° C. for 16 hrs under N₂. The reaction solution was cooled down to 20~25° C. and then quenched with sat. aq. NaHCO₃ (7.7 L) dropwise using peristaltic pump (300 mL/min) under N₂. The mixture was stirred at 20~25° C. for 30 min under N₂. The solution was extracted with Ethyl acetate (5 L×2). The organic phase was washed with brine (5 L×1), dried over Na₂SO₄, filtered and evaporated under reduced pressure to give 5 kg crude. The residue was purified by MPLC (SiO₂, Petroleum ether/Ethyl acetate=1/0 to 10/1) to give 2.64 kg of 3.4 (69.03%) as a yellow oil.

Synthesis of 3.5. Charge DCM (5.4 L) to reactor at 25° C. 7-(tert-butyl) 1-ethyl (R,E)-6-((tert-butoxycarbonyl)amino) hept-2-enedioate (3.4, 2.6 kg, 7.27 mol, 1 eq) in DCM (5 L) was added to reactor at 25° C. TFA (4.98 kg, 43.64 mol, 3.24 L, 6 eq) was added dropwise to the solution (50 mL/min) under N₂. The mixture was stirred at 25° C. for 2 hrs under N₂. NaHCO₃ (6.11 kg, 72.71 mol, 2.83 L, 10 eq) in H₂O (13 L) was added dropwise to the solution (100 mL/min) under N₂. The mixture was stirred at 25° C. for 16 hrs under N₂. The reaction mixture was extracted with DCM (8 L×2). The combined organic layers were washed with brine (8 L), dried over Na₂SO₄, filtered and concentrated under reduced pressure to give a residue (1.477 kg). The residue was purified by column chromatography (SiO₂, Petroleum ether/Ethyl acetate=1/0 to 1/1) to give 876 g of 3.5 (46.8%) as a brown oil.

Synthesis of 3.6. The reaction was set up in 6 batches in parallel. To a solution of Pd/C (12.70 g, 11.93 mmol, 10% purity) in MeOH (889 mL) was added tert-butyl (2R)-5-(2-ethoxy-2-oxoethyl) pyrrolidine-2-carboxylate (3.5, 127 g, 493.54 mmol, 1 eq) and formaldehyde (801.13 g, 9.87 mol, 734.99 mL, 37% purity, 20 eq) at 25° C. Then the mixture was stirred at 25° C. for 16 hrs under H₂ (balloon). Six batches were combined for work-up. The catalyst Pd/C was removed by filtration over celite with MeOH (3×2 L) rinsing and the filtrated was concentrated under reduced pressure to give 1.25 kg of 3.6 (crude) as a yellow oil.

Synthesis of 3.7. To a solution of tert-butyl(2R)-5-(2-ethoxy-2-oxoethyl)-1-methylpyrrolidine-2-carboxylate (3.6, 1.04 kg, 3.83 mol, 1 eq) in THF (6.24 L) was added dropwise LiHMDS (1 M, 8.43 L, 2.2 eq) at −60° C.~−78° C. and the mixture was stirred at −60° C.~−78° C. for 2 hrs under N₂. Then N-diazo-2,4,6-triisopropyl-benzenesulfonamide (1.78 kg, 5.75 mol, 1.5 eq) in THF (14.56 L) was added dropwise to the solution at −60° C.~−78° C. and stirred at −60° C.~−78° C. for 10 mins. The reaction was quenched by AcOH (920.64 g, 15.33 mol, 877.63 mL, 4 eq) at −60° C.~−78° C. and the mixture was stirred at 25° C. for 16 hrs. The reaction was concentrated, poured into 10% aq. Na₂CO₃ (10 L) and extracted with Ethyl acetate (5 L×2). The combined organic phase was washed with brine (5 L×5), dried over anhydrous sodium sulfate, filtered and concentrated under vacuum to give a residue. The residue was purified by column chromatography (SiO₂, Petroleum ether/Ethyl acetate=I/O to 10/1) to give 656 g of 3.7 (54.67%) as a yellow oil.

Synthesis of 3.8. The reaction was set up in 8 batches in parallel. To a solution of Pd/C (8.90 g, 8.36 mmol, 10% purity,) in THF (890 mL) was added tert-butyl (2R)-5-((R)-1-azido-2-ethoxy-2-oxoethyl)-1-methylpyrrolidine-2-carboxylate (3.7, 89.00 g, 284.92 mmol, 1 eq) at 20° C. and the mixture was stirred at 20° C. under H₂ (15 Psi) for 48 hrs. Eight batches were combined for work-up. The mixture was purged with a stream of N₂, filtered over celite with THF (10 L) rinsing and concentrated under reduced pressure to give 610 g of 3.8 (93.4%) as a yellow oil and used to next step directly without purification.

Synthesis of 3.10. The reaction was set up in 6 batches in parallel. To a solution of tert-butyl(2R)-5-((R)-1-amino-2-ethoxy-2-oxoethyl)-1-methylpyrrolidine-2-carboxylate (3.8, 100.00 g, 349.20 mmol, 1 eq) in ACN (800 mL) was added 2-benzyloxybenzoic acid (3.9, 79.70 g, 349.20 mmol, 1 eq) and DIEA (135.40 g, 1.05 mol, 182.47 mL, 3 eq) at 25° C., and then T4P (377.41 g, 523.80 mmol, 50% purity, 1.5 eq) was added dropwise to the solution at 0° C. and the mixture was stirred at 25° C. for 2 hrs. Six batches were combined for work-up. The reaction mixture was quenched with H₂O (4.5 L) and extracted with Ethyl acetate (750 mL×3). The combined organic layers were washed with NaHCO₃ (aq.) (1.3 L×4), dried over Na₂SO₄, filtered and concentrated under reduced pressure to give 978 g of 3.10 (93.99%) as a brown oil, which was used to next step directly without purification.

Synthesis of 3.11A. The reaction was set up in 8 batches in parallel. To a solution of tert-butyl(2R)-5-((R)-1-(2-(benzyloxy)benzamido)-2-ethoxy-2-oxoethyl)-1-methylpyrrolidine-2-carboxylate (3.10, 120 g, 241.65 mmol, 1 eq) in THF (1.2 L) was added LiBH₄ (2 M, 241.65 mL, 2 eq) at 0° C. and the mixture was stirred at 30° C. for 2 hrs under N₂. The reaction was quenched by addition of 1M NaOH (240 mL), stirred for an additional 10 min, and then diluted with water (1 L). Eight reaction mixtures were combined, extracted with Ethyl acetate (5 L×2) and washed with brine (5 L×4). The combined organic layer was dried over Na₂SO₄, filtered, and evaporated to give 841 g crude product. The residue was purified by prep-HPLC (column: Welch Xtimate C18 250*100 mm*10 um; mobile phase: [H₂O (10 mM NH₄HCO₃)-ACN]; gradient: 45%-70% B over 18.0 min) to give 331 g of 3.11A (37.66% yield) and 242 g of 3.11B (27.53%) as yellow oil. (ES, m/z): [M+1]+455.2.

Synthesis of 3.12A. The reaction was set up in 2 batches in parallel. To a solution of tert-butyl(2R,5R)-5-((R)-1-(2-(benzyloxy)benzamido)-2-hydroxyethyl)-1-methylpyrrolidine-2-carboxylate (3.11A, 160.5 g, 353.09 mmol, 1 eq) in EtOH (1.6 L) was added Pd/C (16.05 g, 15.08 mmol, 10% purity) at 25° C., and then the mixture was stirred at 25° C. for 3 hrs under H₂ (balloon). Two batches were combined for work-up. The mixture was purged with a stream of N₂, filtered over celite with DCM (2 L) rinsing and evaporated to give 262.5 g of 3.12A (crude, de value: 87.99%) as a yellow oil, which was used to next step directly without purification. (ES, m/z): [M+1]⁺365.1.

Synthesis of 3.13A. The reaction was set up in 4 batches in parallel. To a solution of tert-butyl(2R,5R)-5-((R)-2-hydroxy-1-(2-hydroxybenzamido)ethyl)-1-methylpyrrolidine-2-carboxylate (3.12A, 67.4 g, 184.94 mmol, 1 eq) in THF (674 mL) was added methoxycarbonyl-(triethylammonio) sulfonyl-azanide (66.11 g, 277.42 mmol, 1.5 eq) at 20° C. and the mixture was stirred at 60° C. for 3 hrs. Four batches were combined for work-up. The reaction mixture was quenched by addition of H₂O (2.5 L) at 25° C. and then extracted with Ethyl acetate (1 Lx 2). The combined organic layers were washed with brine (1 L×3), dried over Na₂SO₄, filtered and concentrated under reduced pressure to give a residue. The residue was purified by column chromatography (SiO₂, Petroleum ether/Ethyl acetate=I/O to 10/1) to give 184.5 g of 3.13A (70.77% yield, de value: 86.02%) as a white solid. (ES, m/z): [M+1]⁺347.1.

Synthesis of 3.14A. The reaction was set up in 9 batches in parallel. To a solution of tert-butyl(2R,5R)-5-((R)-2-(2-hydroxyphenyl)-4,5-dihydrooxazol-4-yl)-1-methylpyrrolidine-2-carboxylate (3.13A, 20 g, 57.73 mmol, 1 eq) in dioxane (200 mL) was added P₂S₅ (6.42 g, 28.87 mmol, 3.07 mL, 0.5 eq) and the mixture was stirred at 110° C. for 48 hrs. The reaction mixture was filtered and the organic layer was quenched by addition of NaHCO₃ (1 L) at 20° C. The suspension was then filtered and the filter cake was collected. The filtrate was extracted with DCM (300 mL×2) and the combined organic layers were washed with brine (100 mL), dried over Na₂SO₄, filtered and concentrated under reduced pressure to give a residue. The residue and filter cake from 9 batches were combined and dissolved in DCM (180 mL), which was purified by column chromatography (SiO₂, Petroleum ether/Ethyl acetate=1:0 to 10:1) to give 3.14A (45.76 g, 24.29% yield, de value: 100%) as a white solid. (ES, m/z): [M+1]⁺363.1.

Synthesis of I-3. The reaction was set up in 7 batches in parallel. To a solution of tert-butyl(2R,5R)-5-[(4R)-2-(2-hydroxyphenyl)-4,5-dihydrothiazol-4-yl]-1-methyl-pyrrolidine-2-carboxylate (3.14A, 6.5 g, 17.93 mmol, 1 eq) in anisole (19.39 g, 179.32 mmol, 19.49 mL, 10 eq) was added formic acid (130 mL) and the mixture was stirred at 60° C. for 3 hrs. Seven batches were combined for work-up. The reaction mixture was concentrated under reduced pressure to give a residue (48 g). The residue was purified by prep-HPLC (column: Welch Xtimate C18 250*100 mm*10 um; mobile phase: [H₂O (10 mM NH₄HCO₃)-ACN]; gradient: 10%-40% B over 18.0 min) to give I-3 (25.2 g, 65.14% yield; de value: 100%) as a yellow solid. (ES, m/z): [M+1]⁺307.1.

I-3: ¹H NMR (400 MHz, chloroform-d) δ=7.46-7.33 (m, 2H), 6.99 (d, J=8.2 Hz, 1H), 6.90 (t, J=7.4 Hz, 1H), 5.15 (dt, J=3.4, 9.4 Hz, 1H), 4.03 (br t, J=8.4 Hz, 1H), 3.96 (br dd, J=5.2, 7.8 Hz, 1H), 3.55 (dd, J=9.4, 11.0 Hz, 1H), 3.22-3.12 (m, 1H), 2.92 (s, 3H), 2.41-2.19 (m, 2H), 2.17-2.06 (m, 1H), 1.97-1.85 (m, 1H).

I-3: ¹H NMR (400 MHz, DMSO-d₆) δ=13.12-12.34 (m, 1H), 7.48-7.34 (m, 2H), 7.01-6.88 (m, 2H), 4.91 (dt, J=2.8, 9.6 Hz, 1H), 3.66-3.59 (m, 1H), 3.53 (dd, J=9.4, 10.8 Hz, 1H), 3.42 (td, J=3.6, 8.8 Hz, 1H), 3.19 (t, J=10.4 Hz, 1H), 2.45 (s, 3H), 2.10 (qd, J=9.4, 12.4 Hz, 1H), 1.99-1.85 (m, 1H), 1.80-1.68 (m, 1H), 1.62-1.48 (m, 1H).

I-3: ¹H NMR (400 MHz, methanol-d₄) δ=7.50 (dd, J=1.6, 7.8 Hz, 1H), 7.42 (ddd, J=1.6, 7.2, 8.4 Hz, 1H), 6.99 (dd, J=0.8, 8.4 Hz, 1H), 6.97-6.91 (m, 1H), 5.26 (dt, J=3.6, 9.4 Hz, 1H), 4.07 (dt, J=3.8, 7.2 Hz, 1H), 3.98 (dd, J=5.6, 8.4 Hz, 1H), 3.68 (dd, J=9.2, 11.4 Hz, 1H), 3.25 (dd, J=9.4, 11.4 Hz, 1H), 3.05 (s, 3H), 2.41-2.24 (m, 2H), 2.18-2.08 (m, 1H), 2.07-1.96 (m, 1H).

### Example 4. (2R, 5S)-5-((R)-2-(2-hydroxyphenyl)-4,5-dihydrothiazol-4-yl)-1-methylpyrrolidine-2-carboxylic acid

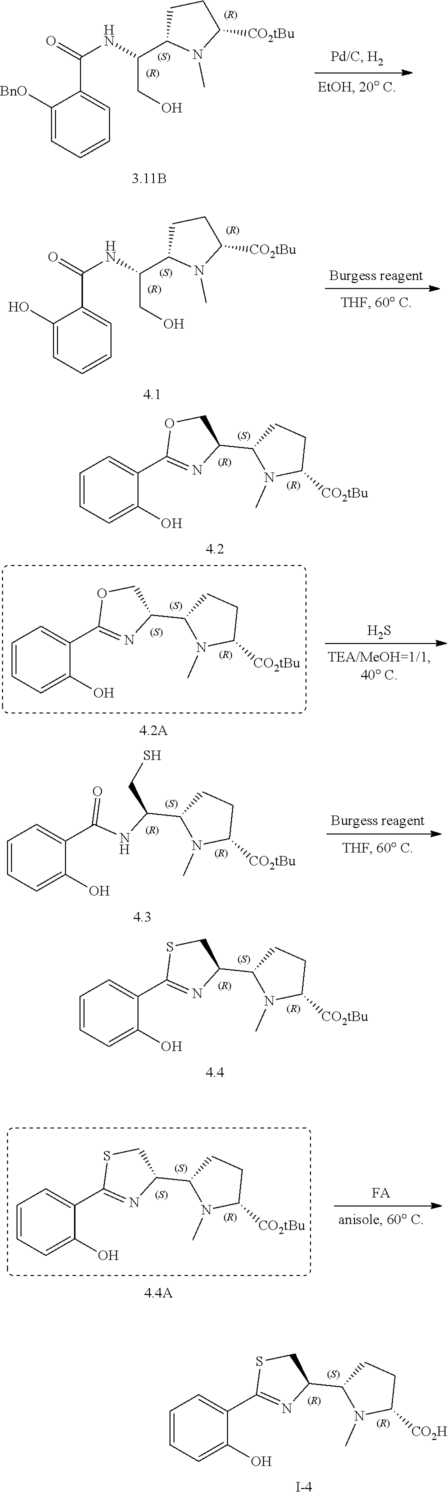

Synthesis of 4.1. The reaction was set up in 2 batches in parallel. A mixture of tert-butyl(2R,5S)-5-((R)-1-(2-(benzyloxy)benzamido)-2-hydroxyethyl)-1-methylpyrrolidine-2-carboxylate (3.11B, 116 g, 255.19 mmol, 1 eq) in EtOH (1160 mL) was degassed and purged with N₂ for 3 times, and then Pd/C (11.60 g, 10.90 mmol, 10% purity) was added. The reaction was stirred at 20° C. under H₂ atmosphere (balloon, 15 Psi) for 3 hrs. Two batches were combined for work-up. The mixture was purged with a stream of N₂, filtered over celite with DCM (2 L) rinsing and evaporated to give 186 g of 4.1 (crude) as a yellow oil, which was used to next step directly without purification. (ES, m/z): [M+1] ⁺365.1.

Synthesis of 4.2. The reaction was set up in 3 batches in parallel. To a solution of tert-butyl(2R,5S)-5-((R)-2-hydroxy-1-(2-hydroxybenzamido)ethyl)-1-methylpyrrolidine-2-carboxylate (4.1, 64 g, 175.61 mmol, 1 eq) in THF (640 mL) was added Burgess reagent (62.78 g, 263.42 mmol, 1.5 eq) and the mixture was stirred at 60° C. for 2 hrs. Three batches were combined for work-up. The combined reaction mixture was diluted with H₂O (1.93 L) and extracted with Ethyl acetate (1 L×3). The combined organic layers were washed with brine (1 L), dried over Na₂SO₄, filtered and concentrated under reduced pressure to give a residue. The residue was purified by flash silica gel chromatography (ISCO®; 300 g SepaFlash® Silica Flash Column, Eluent of 0~10% Ethyl acetate/Petroleum ether gradient @ 180 mL/min) to give 141 g of 4.2 (4.2:4.2A=3:1) (73.80% yield, de value: 30.24%) as a yellow oil. (ES, m/z): [M+1]⁺347.1.

Synthesis of 4.3. The reaction was set up in 84 batches in parallel. tert-butyl (2R,5S)-5-((R)-2-(2-hydroxyphenyl)-4,5-dihydrooxazol-4-yl)-1-methylpyrrolidine-2-carboxylate (4.2, 1.5 g, 4.33 mmol, 1 eq) was dissolved in TEA (30 mL) and MeOH (30 mL) in a round bottom flask. H₂S gas was then bubbled through the stirring solution for 20 min, after which the reaction mixture was transferred to a 100 mL autoclave and warmed to 40° C. The reaction was stirred at 40° C. for 56 hrs. 84 batches were combined for work-up. The combined reaction mixture was concentrated under reduced pressure to give 138 g of 4.3 (crude) as a brown oil and used into next step without further purification. (ES, m/z): [M+1]⁺381.1.

Synthesis of 4.4. The reaction was set up in 5 batches in parallel. To a solution of tert-butyl(2R,5S)-5-((R)-1-(2-hydroxybenzamido)-2-mercaptoethyl)-1-methylpyrrolidine-2-carboxylate (4.3, 19.77 g, 51.96 mmol, 1 eq) in THF (200 mL) was added Burgess Reagent (24.76 g, 103.92 mmol, 2 eq) under N₂. The mixture was stirred at 60° C. for 16 hrs. Five batches were combined for work-up. The combined reaction mixture was diluted with H₂O (1 L) and extracted with ethyl acetate (500 mL×3). The combined organic layers were washed with brine (500 mL), dried over Na₂SO₄, filtered and concentrated under reduced pressure to give a residue. The residue was purified by flash silica gel chromatography (16 batches, ISCO®; 330 g*2 SepaFlash® Silica Flash Column, Eluent of 0~5% Ethylacetate/Petroleum ether gradient @ 150 mL/min) to 42 g of 4.4 (de value: 85.62%) and 3 g of 4.4A (de value: 75.12%) as yellow solid. (ES, m/z): [M+1]⁺363.1.

Synthesis of I-4. The reaction was set up in 10 batches in parallel. To solution of tert-butyl(2R,5S)-5-((R)-2-(2-hydroxyphenyl)-4,5-dihydrothiazol-4-yl)-1-methylpyrrolidine-2-carboxylate (4.4, 4 g, 11.03 mmol, 1 eq) in anisole (11.93 g, 110.35 mmol, 11.99 mL, 10 eq) was added FA (80 mL). The reaction mixture was stirred at 60° C. for 3 hrs. Ten batches were combined for work-up. The reaction mixture was concentrated under reduced pressure to give a residue. The residue was purified by prep-HPLC (neutral condition; column: WePure Biotech XP tC18 250*70*10 um; mobile phase: [H₂O (10 mM NH₄HCO₃)-ACN]; gradient: 20%-50% B over 20.0 min) to give 27 g of I-4 (81.65% yield, de value: 100%) as a light yellow solid. (ES, m/z): [M+1]⁺307.1.

I-4: ¹H NMR (400 MHz, chloroform-d) δ=7.47-7.34 (m, 2H), 7.01 (d, J=8.4 Hz, 1H), 6.90 (t, J=7.6 Hz, 1H), 5.05-4.80 (m, 1H), 3.51 (dd, J=4.8, 8.4 Hz, 1H), 3.47-3.40 (m, 1H), 3.31-3.20 (m, 1H), 3.19-3.09 (m, 1H), 2.76 (s, 3H), 2.33-2.17 (m, 2H), 2.12-2.00 (m, 1H), 1.76 (qd, J=9.0, 12.8 Hz, 1H).

I-4: ¹H NMR (400 MHz, DMSO-d₆) δ=13.05-12.01 (m, 1H), 7.46-7.37 (m, 2H), 7.02-6.89 (m, 2H), 4.99 (dt, J=5.8, 8.8 Hz, 1H), 3.47 (dd, J=9.4, 11.4 Hz, 1H), 3.30 (dd, J=8.4, 11.4 Hz, 1H), 3.15-3.00 (m, 2H), 2.44 (s, 3H), 2.07-1.94 (m, 1H), 1.87-1.71 (m, 2H), 1.65-1.51 (m, 1H).

I-4: ¹H NMR (400 MHz, methanol-d₄) δ=7.48 (dd, J=1.6, 7.8 Hz, 1H), 7.41 (ddd, J=1.6, 7.4, 8.4 Hz, 1H), 7.01-6.96 (m, 1H), 6.95-6.89 (m, 1H), 5.11 (q, J=8.8 Hz, 1H), 3.92 (dd, J=3.8, 7.8 Hz, 1H), 3.66 (dt, J=7.2, 9.4 Hz, 1H), 3.58 (dd, J=8.6, 11.4 Hz, 1H), 3.22 (dd, J=8.8, 11.4 Hz, 1H), 3.02 (s, 3H), 2.48-2.37 (m, 1H), 2.36-2.24 (m, 2H), 1.97-1.82 (m, 1H).

### Example 5. (2R,5S)-5-((R)-2-(2-hydroxyphenyl)-4,5-dihydrothiazol-4-yl)-N,1-dimethyl pyrrolidine-2-carboxamide

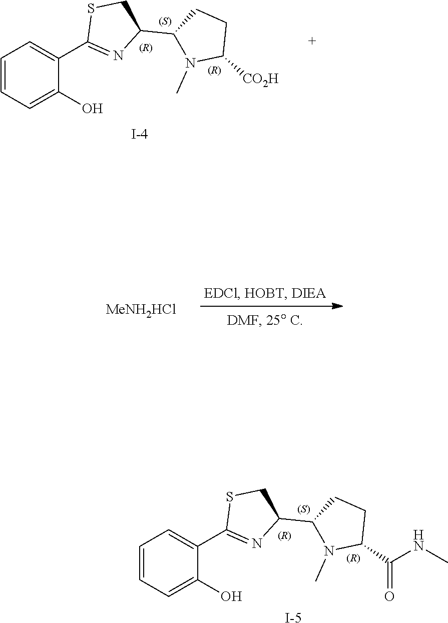

Synthesis of I-5. To a solution of (2R,5S)-5-((R)-2-(2-hydroxyphenyl)-4,5-dihydrothiazol-4-yl)-1-methylpyrrolidine-2-carboxylic acid (I-4, 70 mg, 228.47 umol, 1 eq) in DMF (0.5 mL) was added EDCI (52.56 mg, 274.17 umol, 1.2 eq), HOBt (37.05 mg, 274.17 umol, 1.2 eq), DIEA (88.58 mg, 685.42 umol, 119.39 uL, 3 eq) and methanamine hydrochloride (5.1, 23.14 mg, 342.71 umol, 342.71 uL, 1.5 eq). The mixture was stirred at 25° C. for 4 hrs. The residue was purified by prep-HPLC (column: Waters Xbridge BEH C18 100*30 mm*10 um; mobile phase: [water (NH₄HCO₃)-ACN]; B %: 40%-60%, 8 min) to give 22.18 mg of I-5 (30%, stereo chemical ratio: 5.8:94.2) as a white solid. (ES, m/z): [M+1]+320.1.

I-5: ¹H NMR (400 MHz, methanol-d₄) δ=7.44 (dd, J=0.8, 7.6 Hz, 1H), 7.40-7.32 (m, 1H), 7.00-6.86 (m, 2H), 4.95 (dt, J=5.2, 8.8 Hz, 1H), 3.52 (dd, J=9.2, 11.2 Hz, 1H), 3.36-3.33 (m, 1H), 3.22-3.08 (m, 2H), 2.68 (s, 3H), 2.49 (s, 3H), 2.20-2.10 (m, 1H), 1.94 (br dd, J=5.6, 12.0 Hz, 1H), 1.84-1.68 (m, 2H).

### Example 6. (2R,5R)-5-((R)-2-(2-hydroxy-4-(2-(2-methoxyethoxy) ethoxy)phenyl)-4,5-dihydrooxazol-4-yl)-1-methylpyrrolidine-2-carboxylic acid

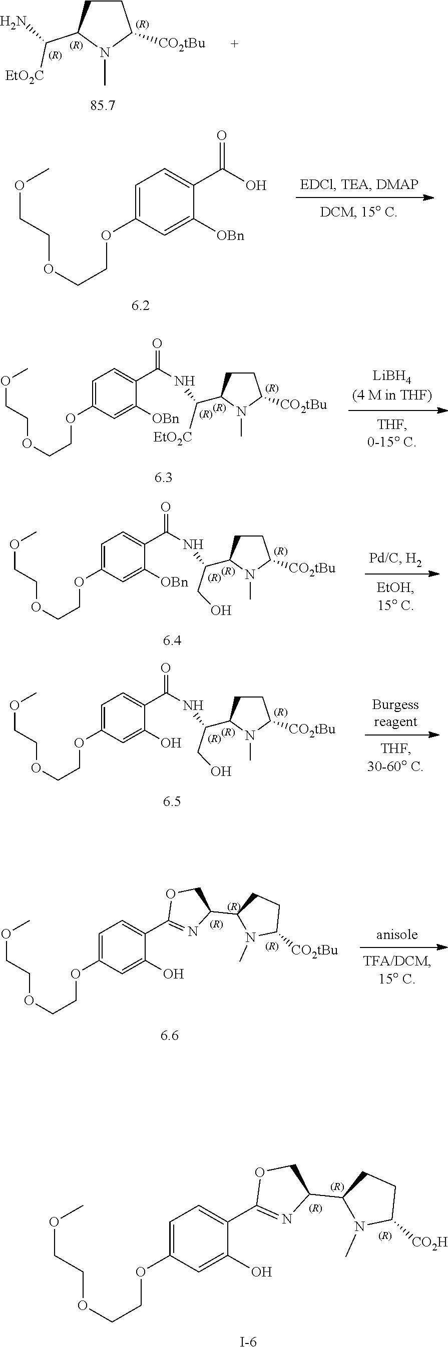

### Synthesis of 6.2

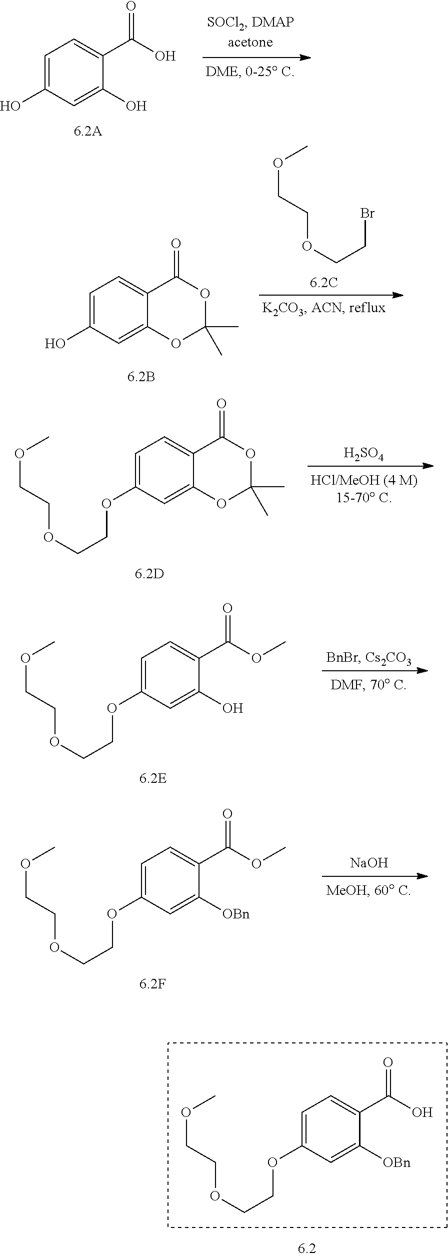

Synthesis of 6.2B. To a solution of 2,4-dihydroxybenzoic acid (6.2A, 52 g, 337.40 mmol, 1 eq) in dimethoxyethane (200 mL) was added DMAP (2.47 g, 20.24 mmol, 0.06 eq) and acetone (29.39 g, 506.10 mmol, 37.21 mL, 1.5 eq). Thionyl chloride (80.28 g, 674.80 mmol, 48.95 mL, 2 eq) was added to the mixture dropwise. The mixture was stirred at 0° C. for 1 hr and then stirred at 25° C. for 5 hrs. Sat. NaHCO₃ was added to the mixture until pH=5, then the mixture was extracted with ethyl acetate (2×500 mL) and H₂O (500 mL×2). The organic layers were collected, dried over Na₂SO₄, filtered and concentrated under reduced pressure to give a residue. The residue was triturated with dichloromethane to afford 12.5 g of 6.2B as a yellow solid which was used directly without further purification. (ES, m/z): [M+1]⁺195.1.

Synthesis of 6.2D. To a solution of 7-hydroxy-2,2-dimethyl-4H-benzo[d][1,3]dioxin-4-one (6.2B, 11.45 g, 58.96 mmol, 1 eq) and 1-bromo-2-(2-methoxyethoxy) ethane (6.2C, 11.87 g, 64.86 mmol, 1.1 eq) in ACN (110 mL) was added K₂CO₃ (24.45 g, 176.89 mmol, 3 eq). The mixture was stirred at 90° C. for 5 hrs. The suspension was filtered through a pad of Celite and the filter cake was washed with ethyl acetate (3×50 mL). The combined filtrates were concentrated to dryness to give a residue. The residue was purified by column chromatography (SiO₂, Petroleum ether:Ethyl acetate=99:1 to 10:1) to give 14.05 g of 6.2D (80%) as a light yellow oil. (ES, m/z): [M+1]⁺297.1.

Synthesis of 6.2E. A solution of 7-[2-(2-methoxyethoxy) ethoxy]-2,2-dimethyl-1,3-benzodioxin-4-one (6.2D, 7 g, 23.62 mmol, 1 eq) in HCl/MeOH (4 M, 5.91 mL, 1 eq) was stirred for 0.5 hr at 15° C. Then H₂SO₄ (6.95 g, 70.83 mmol, 3.78 mL, 3 eq) was added to the mixture, and the mixture was stirred for 12 hrs at 70° C. The reaction mixture was diluted with H₂O (100 mL) and extracted with ethyl acetate (100 mL×3). The combined organic layers were washed with brine (100 mL), dried over Na₂SO₄, filtered and concentrated under reduced pressure to give 6.38 g of 6.2E as a brown liquid which was used directly without further purification. (ES, m/z): [M+1]⁺271.1.

Synthesis of 6.2F. To a solution of methyl 2-hydroxy-4-[2-(2-methoxyethoxy) ethoxy]benzoate (6.2E, 6.38 g, 23.61 mmol, 1 eq) in DMF (60 mL) was added Cs₂CO₃ (15.38 g, 47.21 mmol, 2 eq) and bromomethylbenzene (6.06 g, 35.41 mmol, 4.21 mL, 1.5 eq). The mixture was stirred at 70° C. for 2 hrs. The suspension was filtered through a pad of Celite and the pad cake was washed with ethyl acetate (150 mL×3) and H₂O (150 mL×3). The combined filtrates were concentrated to dryness to give a residue. The residue was purified by column chromatography (SiO₂, Petroleum ether/Ethyl acetate=99/1 to 10/1) to give 8.51 g of 6.2F as a yellow liquid which was used directly without further purification. (ES, m/z): [M+1]+361.2.

Synthesis of 6.2. To a solution of methyl 2-benzyloxy-4-[2-(2-methoxyethoxy) ethoxy]benzoate (6.2F, 3 g, 8.32 mmol, 1 eq) in MeOH (5 mL) and H₂O (5 mL) was added NaOH (832.35 mg, 20.81 mmol, 2.5 eq). The mixture was stirred at 60° C. for 3 hrs. The solution was cooled to 15° C. and HCl (0.1 M) was added to the mixture until pH=3-4. The suspension was extracted with ethyl acetate (50 mL×3). The combined organic layer was washed with brine (30 mL), dried over Na₂SO₄, filtered and concentrated under reduced pressure to give a residue to give 2.38 g of 6.2 as light yellow oil which was used directly without further purification. (ES, m/z): [M+1]+347.1.

Synthesis of 6.3. A solution of 2-(benzyloxy)-4-(2-(2-methoxyethoxy) ethoxy)benzoic acid (6.2, 2.31 g, 6.66 mmol, 1.2 eq), EDCI (1.28 g, 6.66 mmol, 1.2 eq), TEA (674.21 mg, 6.66 mmol, 927.38 uL, 1.2 eq) in DCM (23 mL) was added a catalytic amount of DMAP (67.83 mg, 555.23 umol, 0.1 eq) and (2R,5R)-tert-butyl 5-((R)-1-amino-2-ethoxy-2-oxoethyl)-1-methylpyrrolidine-2-carboxylate (85.7, 1.59 g, 5.55 mmol, 1 eq) at 15° C. The reaction mixture was stirred at 15° C. for 16 hrs. The reaction mixture was diluted with water (30 mL) and extracted with DCM (20 mL×3). The combined organic layers were dried over Na₂SO₄, filtered and concentrated under reduced pressure to give a residue. The residue was purified by column chromatography (SiO₂, Petroleum ether/Ethyl acetate=1/0 to 63/37) to give 2.53 g of 6.3 (74%, de value: 99.36%) as a yellow oil. (ES, m/z): [M+1]+615.5.

Synthesis of 6.4. To a solution of (2R,5R)-tert-butyl 5-((R)-1-(2-(benzyloxy)-4-(2-(2-methoxyethoxy) ethoxy)benzamido)-2-ethoxy-2-oxoethyl)-1-methylpyrrolidine-2-carboxylate (6.3, 2.3 g, 3.74 mmol, 1 eq) in THF (23 mL) was added LiBH₄ (4 M, 1.40 mL, 1.5 eq) at 0° C. and stirred for 4 hrs at 15° C. Another batch of LiBH₄ (4 M, 467.69 uL, 0.5 eq) was added to the reaction and stirred for 12 hrs at 15° C. The reaction was quenched by addition of 1M NaOH (120 mL), stirred for an additional 10 min, and then diluted with water (50 mL) and extracted with ethyl acetate (150 mL×3). The combined organic layers were dried over Na₂SO₄, filtered, and concentrated to give a residue, which was purified by column chromatography (SiO₂, Petroleum ether/Ethyl acetate=1/0 to 41/59) to give 1.51 g of 6.4 (70%, de value: 95%) as a colorless oil. (ES, m/z): [M+1]+573.4.

Synthesis of 6.5. A mixture of (2R,5R)-tert-butyl 5-((R)-1-(2-(benzyloxy)-4-(2-(2-methoxyethoxy) ethoxy)benzamido)-2-hydroxyethyl)-1-methylpyrrolidine-2-carboxylate (6.4, 1.51 g, 2.64 mmol, 1 eq) in EtOH (15 mL) was degassed and purged with N₂ for 3 times, and treated with a catalytic amount of Pd/C (500 mg, 10% purity) under H₂ atmosphere (balloon). The reaction mixture was stirred 16 hrs at 15° C. The mixture was filtered over celite with EtOH rinsing and concentrated to give 1.47 g of 6.5 (crude) as a colorless oil. (ES, m/z): [M+1]+483.2.

Synthesis of 6.6. To a solution of (2R, 5R)-tert-butyl 5-((R)-2-hydroxy-1-(2-hydroxy-4-(2-(2-methoxyethoxy) ethoxy)benzamido)ethyl)-1-methylpyrrolidine-2-carboxylate (6.5, 1.17 g, 2.42 mmol, 1 eq) in THF (18 mL) was added Burgess reagent (866.67 mg, 3.64 mmol, 1.5 eq). The reaction mixture was stirred for 0.5 hr at 30° C. and for 0.5 hr at 60° C. The reaction mixture was diluted with water (50 mL) and extracted with Ethyl acetate (50 mL×3). The combined organic layers were dried over Na₂SO₄, filtered and concentrated under reduced pressure to give a residue. The residue was purified by column chromatography (SiO₂, Petroleum ether/Ethyl acetate=1/0 to 65/35) to give 680 mg of 6.6 (60%, de value: 90.6%) as a colorless oil. (ES, m/z): [M+1]+465.2.

Synthesis of I-6. To a solution of (2R,5R)-tert-butyl 5-((R)-2-(2-hydroxy-4-(2-(2-methoxyethoxy) ethoxy)phenyl)-4,5-dihydrooxazol-4-yl)-1-methylpyrrolidine-2-carboxylate (6.6, 45 mg, 93.63 umol, 1 eq) in DCM (0.5 mL) and TFA (4.5 mL) was added anisole (101.25 mg, 936.30 umol, 101.76 uL, 10 eq) at 15° C. The mixture was stirred at 15° C. for 16 hrs. The reaction mixture was filtered and concentrated under reduced pressure to give a residue. The crude product was purified by reversed-phase HPLC (column: Waters Xbridge BEH C18 100*30 mm*10 um; mobile phase: [water (NH₄HCO₃)-ACN]; B %: 10%-50%, 8 min) to give 62.6 mg of I-6 (71%, de value: 98%) as a white solid. (ES, m/z): [M+1]⁺409.2.

I-6: ¹H NMR (400 MHz, methanol-d₄) δ=7.60 (d, J=10 Hz, 1H), 6.56-6.51 (m, 2H), 4.86-4.81 (m, 1H), 4.61 (t, J=9.2 Hz, 1H), 4.25-4.15 (m, 3H), 3.91-3.82 (m, 4H), 3.73-3.68 (m, 2H), 3.60-3.57 (m, 2H), 3.38 (s, 3H), 2.94 (s, 3H), 2.30-2.18 (m, 2H), 2.03 (br d, J=4.2 Hz, 1H), 1.82 (br d, J=3.8 Hz, 1H)

### Example 7. (2R,5S)-5-((R)-2-(2-hydroxy-4-(2-(2-methoxyethoxy) ethoxy)phenyl)-4,5-dihydrooxazol-4-yl)-1-methylpyrrolidine-2-carboxylic acid

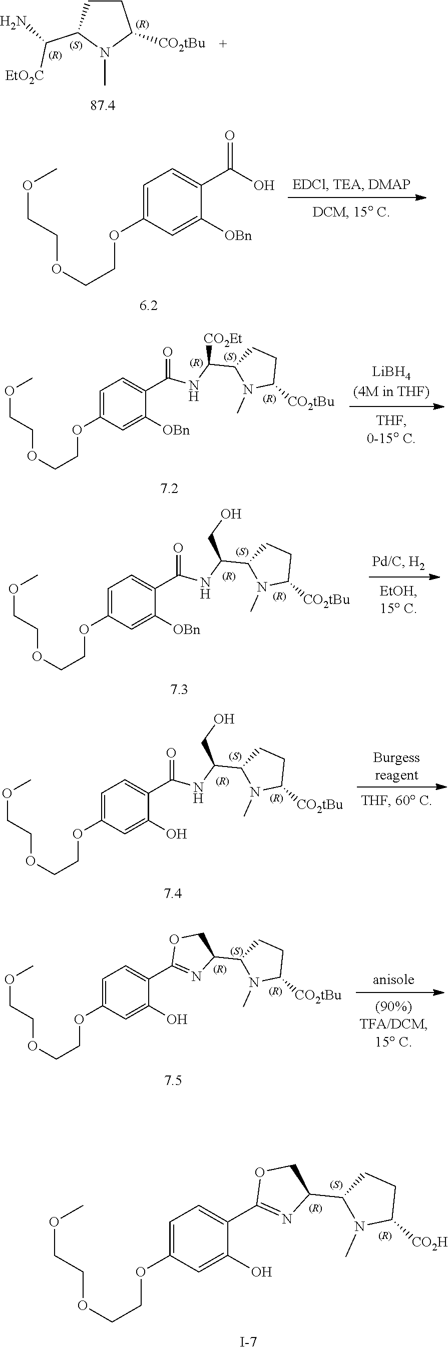

Synthesis of 7.2. A mixture of 2-benzyloxy-4-[2-(2-methoxyethoxy) ethoxy]benzoic acid (6.2, 1.45 g, 4.19 mmol, 1.2 eq), DMAP (42.66 mg, 349.20 umol, 0.1 eq), EDCI (1.00 g, 5.24 mmol, 1.5 eq), TEA (530.03 mg, 5.24 mmol, 729.07 uL, 1.5 eq) in dichloromethane (20 mL) was added tert-butyl(2R,5S)-5-[(1R)-1-amino-2-ethoxy-2-oxo-ethyl]-1-methyl-pyrrolidine-2-carboxylate (87.4, 1 g, 3.49 mmol, 1 eq). The mixture was stirred at 15° C. for 1 hr. The reaction mixture was diluted with water (30 mL) and extracted with dichloromethane 60 mL (20 mL×3). The combined organic layers were dried over Na₂SO₄, filtered and concentrated under reduced pressure to give a residue. The residue was purified by column chromatography (SiO₂, Petroleum ether/Ethyl acetate=1/0 to 63/37) to give 1.8 g of 7.2 (75%) as a yellow oil. (ES, m/z): [M+1]⁺615.3.

Synthesis of 7.3. A solution of tert-butyl(2R,5S)-5-[(1R)-1-[[2-benzyloxy-4-[2-(2-methoxyethoxy) ethoxy]benzoyl]amino]-2-ethoxy-2-oxo-ethyl]-1-methyl-pyrrolidine-2-carboxylate (7.2, 1.6 g, 2.60 mmol, 1 eq) in THF (30 mL) was treated with LiBH₄ (4 M, 976.05 uL, 1.5 eq) at 0° C. and stirred at 15° C. for 4 hrs. The reaction was quenched by addition of 1M NaOH (6 mL), stirred for an additional 10 mins, then diluted with water (10 mL) and extracted with ethyl acetate (20 mL×3). The combined organic layers were dried over Na₂SO₄, filtered and concentrated under reduced pressure to give a residue which was purified by chromatography (SiO₂, Petroleum ether/Ethyl acetate=100/1 to 0/100) to give 921.7 mg of 7.3 (39%) as a yellow oil. (ES, m/z): [M+1]⁺573.3.

Synthesis of 7.4. To a solution (2R,5S)-tert-butyl 5-((R)-1-(2-(benzyloxy)-4-(2-(2-methoxyethoxy) ethoxy)benzamido)-2-hydroxyethyl)-1-methylpyrrolidine-2-carboxylate (7.3, 1.01 g, 1.76 mmol, 1 eq) in ethyl alcohol (30 mL) was added a catalytic amount of Pd/C (100 mg, 10% purity) and the flask was then purged and filled with an H₂ atmosphere at 15° C. under 15 Psi. The reaction mixture was stirred for 2 hrs at 15° C. The catalyst Pd/C was removed by filtration over celite with EtOH (5 mL×3) rinsing. The filtrate was concentrated to give 752.6 mg of 7.4 (crude) as a yellow oil. (ES, m/z): [M+1]⁺483.7.

Synthesis of 7.5. To a solution of tert-butyl(2R, 5S)-5-[(1R)-2-hydroxy-1-[[2-hydroxy-4-[2-(2-methoxyethoxy) ethoxy]benzoyl]amino]ethyl]-1-methyl-pyrrolidine-2-carboxylate (7.4, 552.6 mg, 1.15 mmol, 1 eq) in THF (15 mL) was added Burgess Reagent (682.24 mg, 2.86 mmol, 2.5 eq). The mixture was stirred at 60° C. for 2 hrs. The reaction was concentrated and the crude residue was purified by column chromatography (SiO₂, Petroleum ether/Ethyl acetate=100/1 to 1/1) to give 426.5 mg of 7.5 (80%) as a yellow oil. (ES, m/z): [M+1]⁺465.2.

Synthesis of I-7. To a solution of tert-butyl(2R,5S)-5-[(4R)-2-[2-hydroxy-4-[2-(2-methoxyethoxy) ethoxy]phenyl]-4,5-dihydrooxazol-4-yl]-1-methyl-pyrrolidine-2-carboxylate (7.5, 40 mg, 86.10 umol, 1 eq) in TFA (1.8 mL) and dichloromethane (0.2 mL) was added ANISOLE (9.31 mg, 86.10 umol, 9.36 uL, 1 eq). The reaction mixture was stirred at 15° C. for 6 hrs. The reaction mixture was concentrated under reduced pressure to give a residue. The residue was purified by prep-HPLC (column: Waters Xbridge BEH C18 100*30 mm*10 um; mobile phase: [water (NH₄HCO₃)-ACN]; B %: 5%-35%, 8 min) to give 31.29 mg of I-7 (89%) as a white solid. (ES, m/z): [M+1]⁺409.2.

1-7: ¹H NMR (400 MHz, methanol-d₄) δ=7.55 (d, J=9.6 Hz, 1H), 6.56-6.43 (m, 2H), 4.70-4.57 (m, 1H), 4.45 (t, J=9.2 Hz, 1H), 4.38 (t, J=7.6 Hz, 1H), 4.14 (t, J=4.4 Hz, 2H), 3.83 (t, J=4.4 Hz, 2H), 3.69 (dd, J=4, 6.4 Hz, 2H), 3.56 (dd, J=2.8, 4.8 Hz, 2H), 3.36 (s, 3H), 3.16 (br t, J=7.2 Hz, 1H), 2.94 (q, J=7.2 Hz, 1H), 2.58 (s, 3H), 2.13 (m, 1H), 1.88 (m, 2H), 1.62-1.47 (m, 1H)

### Example 8. (2R,5S)-5-((R)-2-(2-hydroxy-4-(2-(2-methoxyethoxy) ethoxy)phenyl)-4,5-dihydrothiazol-4-yl)-1-methylpyrrolidine-2-carboxylic acid

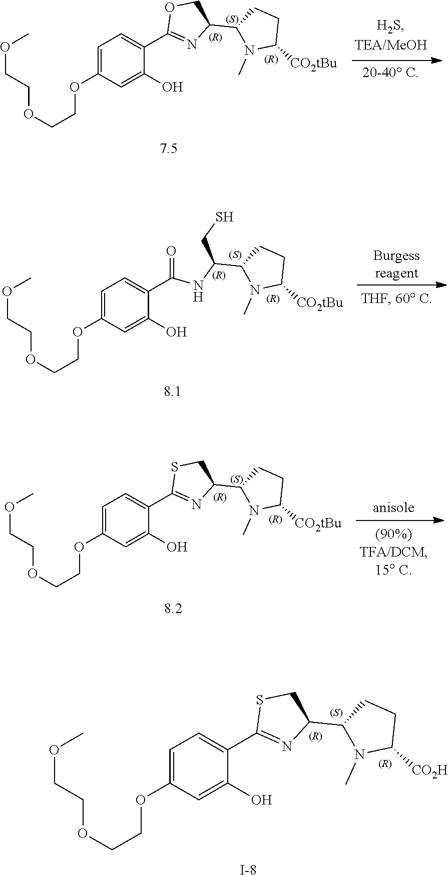

Synthesis of 8.1. A solution of tert-butyl(2R,5S)-5-[(4R)-2-[2-hydroxy-4-[2-(2-methoxyethoxy) ethoxy]phenyl]-4,5-dihydrooxazol-4-yl]-1-methyl-pyrrolidine-2-carboxylate (7.5, 426 mg, 917.02 umol, 1 eq) was dissolved in methanol (5 mL) and TEA (5 mL) at 20° C. in a pressure flask. H₂S gas was then bubbled through the stirring solution for 20 mins, after which the pressure flask was closed and warmed to 40° C. The reaction was stirred at 40° C. for 168 hrs. The reaction was then purged with a stream of Ar by bubbling through the solution for 5 mins to remove H₂S. The solution was concentrated to give 453 mg of 8.1 (crude) as a yellow oil. (ES, m/z): [M+1]⁺499.4.

Synthesis of 8.2. To a solution of tert-butyl(2R,5S)-5-[(1R)-1-[[2-hydroxy-4-[2-(2-methoxyethoxy) ethoxy]benzoyl]amino]-2-sulfanyl-ethyl]-1-methyl-pyrrolidine-2-carboxylate (8.1, 408 mg, 818.24 umol, 1 eq) in THF (12 mL) was added Burgess reagent (487.49 mg, 2.05 mmol, 2.5 eq). The mixture was stirred at 60° C. for 2 hrs. The mixture was concentrated under reduced pressure to give a residue which was purified by chromatography (SiO₂, Petroleum ether/Ethyl acetate=100/1 to 60/40) to give 231 mg of 8.2 (59%) as a yellow oil. (ES, m/z): [M+1]⁺481.4.

Synthesis of I-8. To a solution of tert-butyl(2R,5S)-5-[(4R)-2-[2-hydroxy-4-[2-(2-methoxyethoxy) ethoxy]phenyl]-4,5-dihydrothiazol-4-yl]-1-methyl-pyrrolidine-2-carboxylate (8.2, 210 mg, 436.94 umol, 1 eq) in TFA (9 mL) and dichloromethane (1 mL) was added anisole (472.51 mg, 4.37 mmol, 474.88 uL, 10 eq). The mixture was stirred at 15° C. for 6 hrs. The reaction mixture was concentrated under reduced pressure to give a residue. The residue was purified by prep-HPLC (column: Phenomenex C18 80*40 mm*3 um; mobile phase: [water (NH₄HCO₃)-ACN]; B %: 10%-30%, 8 min) to give 109.41 mg of I-8 (59%) as a white solid. (ES, m/z): [M+1]⁺425.0.

I-8: ¹H NMR (400 MHz, methanol-d₄) δ=7.40 (d, J=8.4 Hz, 1H), 6.58-6.48 (m, 2H), 5.05 (q, J=8.8 Hz, 1H), 4.20-4.12 (m, 2H), 4.00-3.91 (m, 1H), 3.88-3.80 (m, 2H), 3.69 (dd, J=4.4, 6.4 Hz, 3H), 3.61-3.52 (m, 3H), 3.37 (s, 3H), 3.26-3.15 (m, 1H), 3.03 (s, 3H), 2.49-2.39 (m, 1H), 2.39-2.24 (m, 2H), 1.99-1.81 (m, 1H)

### Example 9. ((2R, 5S)-5-((R)-2-(2-hydroxyphenyl)-4,5-dihydrothiazol-4-yl)-1-methylpyrrolidin-2-yl) (piperidin-1-yl)

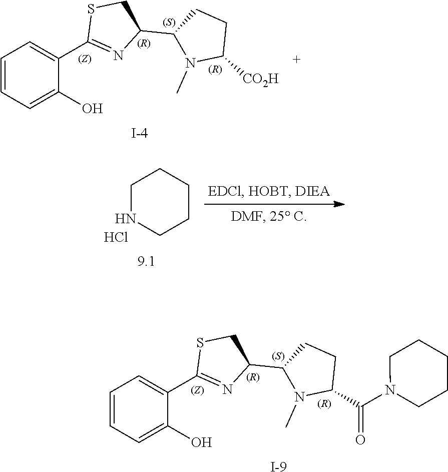

Synthesis of I-9. To a solution of (2R,5S)-5-((R)-2-(2-hydroxyphenyl)-4,5-dihydrothiazol-4-yl)-1-methylpyrrolidine-2-carboxylic acid (I-4, 34.4 mg, 81.83 umol, 1 eq, TFA salt) in DMF (0.5 mL) was added EDCI (18.82 mg, 98.19 umol, 1.2 eq), HOBt (13.27 mg, 98.19 umol, 1.2 eq), DIEA (52.88 mg, 409.13 umol, 71.26 uL, 5 eq) and piperidine hydrochloride (9.1, 11.94 mg, 98.19 umol, 13.85 uL, 1.2 eq, HCl). The mixture was stirred at 25° C. for 0.5 hr. The residue was purified by prep-HPLC (column: C18-1 150*30 mm*5 um; mobile phase: [water (NH₄HCO₃)-ACN]; B %: 50%-80%. 10 min) to give 10.75 mg of I-9 mixture (Stereo chemical ratio: 87:12) as a white solid. 10.75 mg of I-9 mixture was purified by SFC separation (Method 1) to give 6.60 mg of I-9 (61%, stereo chemical ratio: 100%) as a white solid. (ES, m/z): [M+1]⁺374.2.

Method 1: Instrument ChiralPak IH, (250 mm*30 mm, 10 um), Mobile phase: A for CO₂ and B for IPA (0.1% NH₃H₂O); Gradient: B %=40% isocratic elution mode; Flow rate: 70 g/min; Wavelength: 220 nm; Column temperature: 35 degrees centigrade; System back pressure: 120 bar.

1-9: ¹H NMR (400 MHz, methanol-d₄) δ=7.42 (br d, J=7.6 Hz, 1H), 7.35 (br t, J=7.6 Hz, 1H), 6.99-6.84 (m, 2H), 5.08-4.96 (m, 1H), 3.68 (br d, J=3.2 Hz, 2H), 3.61-3.52 (m, 1H), 3.51-3.34 (m, 4H), 3.12-3.00 (m, 1H), 2.40 (s, 3H), 2.17-2.03 (m, 1H), 1.96-1.82 (m, 1H), 1.76-1.51 (m, 8H).

### Example 10. (2R,5R)-5-((R)-2-(2-hydroxy-4-(2-(2-methoxyethoxy) ethoxy)phenyl)-4,5-dihydrothiazol-4-yl)-1-methylpyrrolidine-2-carboxylic acid

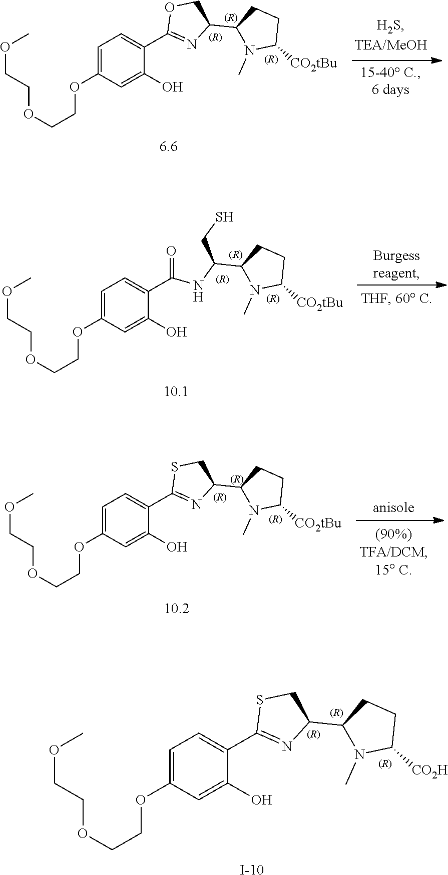

Synthesis of 10.1. (2R,5R)-tert-butyl 5-((R)-2-(2-hydroxy-4-(2-(2-methoxyethoxy) ethoxy)phenyl)-4,5-dihydrooxazol-4-yl)-1-methylpyrrolidine-2-carboxylate (6.6, 413 mg, 889.03 umol, 1 eq) was dissolved in methanol (5 mL) and TEA (5 mL) at 15° C. in a pressure flask. H₂S gas was then bubbled through the stirring solution for 40 mins, after which the pressure flask was closed and warmed to 40° C. The reaction was stirred at 40° C. for 6 days. The reaction was then purged with a stream of Ar by bubbling through the solution for 15 mins to remove H₂S. The reaction mixture was concentrated under reduced pressure to give 465 mg of 10.1 (crude) as a light yellow oil. The crude product was used into the next step without further purification. (ES, m/z): [M+1]⁺499.3.

Synthesis of 10.2. To a solution of (2R,5R)-tert-butyl 5-((R)-1-(2-hydroxy-4-(2-(2-methoxyethoxy) ethoxy)benzamido)-2-mercaptoethyl)-1-methylpyrrolidine-2-carboxylate (10.1, 410 mg, 822.25 umol, 1 eq) in THF (5 mL) was added Burgess reagent (293.93 mg, 1.23 mmol, 1.5 eq). The reaction mixture was stirred at 60° C. for 0.5 hr. The reaction mixture was concentrated under reduced pressure to give a residue. The residue was purified by column chromatography (SiO₂, Petroleum ether/Ethyl acetate=I/O to 78/22) to give 70 mg of 10.2 (17%, de value: 98.58%) as a light yellow oil. (ES, m/z): [M+1] +481.2.

Synthesis of I-10. To a solution of (2R,5R)-tert-butyl 5-((R)-2-(2-hydroxy-4-(2-(2-methoxyethoxy) ethoxy)phenyl)-4,5-dihydrothiazol-4-yl)-1-methylpyrrolidine-2-carboxylate (10.2, 60 mg, 124.84 umol, 1 eq) in dichloromethane (0.5 mL) and TFA (4.5 mL) was added anisole (135.00 mg, 1.25 mmol, 135.68 uL, 10 eq). The mixture was stirred at 15° C. for 16 hrs. The reaction mixture was concentrated under reduced pressure to give a residue. The crude product was purified by reversed-phase HPLC (column: Waters Xbridge BEH C18 100*30 mm*10 um; mobile phase: [water NH₄HCO₃-ACN]; B %: 20%-45%, 8 min). The product was purified by SFC separation (Method 1) to give 45 mg of I-10 (84%, de value: 100%) as a white solid.

Method 1 (Instrument: Waters SFC150AP preparative SFC; Column: ChiralPak IH, 250*30 mm, 10 um) Mobile phase: A for CO₂ and B for IPA (0.1% NH₃H₂O); Gradient: B %=40% isocratic elution mode; Flow rate: 70 g/min; Wavelength: 220 nm; Column temperature: 35° C.; System back pressure: 120 bar.) to give 45 mg of I-10 (84%, de value: 100%) as a white solid.

I-10: ¹H NMR (400 MHz, methanol-d₄) δ=7.38 (d, J=8.4 Hz, 1H), 6.55-6.50 (m, 2H), 5.19 (td, J=5.6, 3.6 Hz, 1H), 4.16 (d, J=4.4 Hz, 2H), 4.08-4.00 (m, 1H), 3.95 (br dd, J=5.6, 7.2 Hz, 1H), 3.84 (br t, J=4.8 Hz, 2H), 3.71-3.67 (m, 2H), 3.63 (dd, J=9.2, 11.2 Hz, 1H), 3.56 (dd, J=2.8, 4.8 Hz, 2H), 3.36 (s, 3H), 3.20 (dd, J=9.2, 11.2 Hz 1H), 3.03 (s, 3H), 2.41-2.21 (m, 2H), 2.18-1.96 (m, 2H).

### Example 11. (2R,5R)-5-((R)-2-(3-hydroxypyridin-2-yl)-4,5-dihydrooxazol-4-yl)-1-methylpyrrolidine-2-carboxylic acid

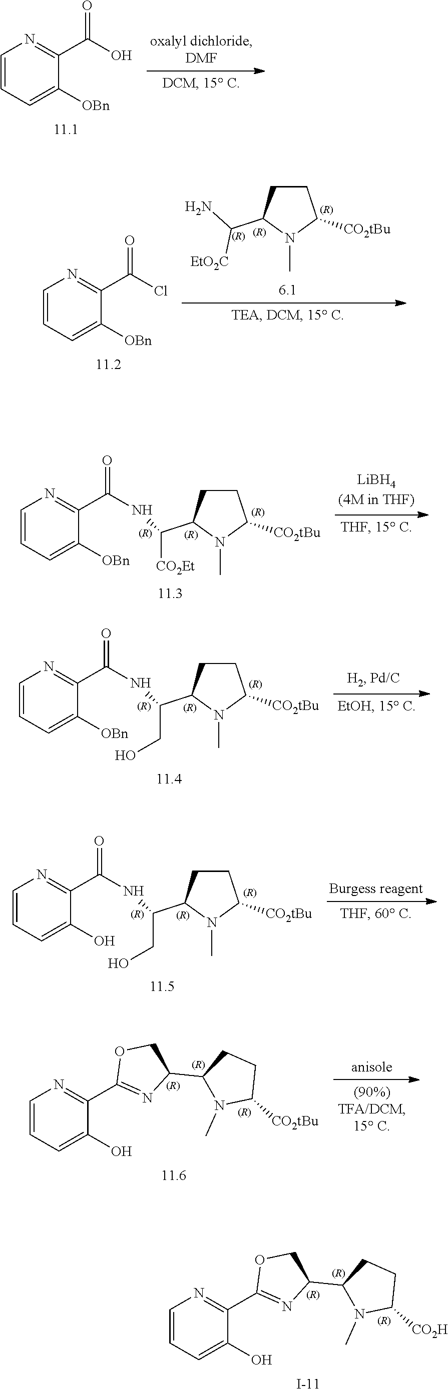

Synthesis of 11.2. To a solution of 3-(benzyloxy) picolinic acid (11.1, 1.57 g, 6.85 mmol, 1 eq) in DCM (31 mL) was added oxalyl dichloride (2.61 g, 20.55 mmol, 1.80 mL, 3 eq) and DMF (5.01 mg, 68.49 umol, 5.27 uL, 0.01 eq). The mixture was stirred at 15° C. for 1 hr. The reaction mixture was filtered and concentrated under reduced pressure to give a residue to give 3.39 g of 11.2 (crude) as a brown solid.

Synthesis of 11.3. To a solution of (2R,5R)-tert-butyl 5-((R)-1-amino-2-ethoxy-2-oxoethyl)-1-methylpyrrolidine-2-carboxylate (85.7, 1.63 g, 5.69 mmol, 1 eq) in dichloromethane (32 mL) was added TEA (1.15 g, 11.38 mmol, 1.58 mL, 2 eq) and stirred at 15° C. for 10 mins. Then 3-(benzyloxy) picolinoyl chloride (11.2, 1.69 g, 6.83 mmol, 1.2 eq) was added. The mixture was stirred at 15° C. for 2 hrs. The reaction mixture was diluted with water (30 mL) and extracted with dichloromethane (30 mL×3). The combined organic layers were dried over Na₂SO₄, filtered and concentrated under reduced pressure to give a residue. The residue was purified by column chromatography (SiO₂, Petroleum ether/Ethyl acetate=I/O to 43/57). The crude product was purified by reversed-phase HPLC (column: Waters Xbridge BEH C18 250*70 mm*10 um; mobile phase: [water (NH₄HCO₃)-ACN]; B %: 40%-70%, 20 min) to give 2.88 g of 11.3 (50%) as a light yellow oil. (ES, m/z): [M+1]+498.4.

Synthesis of 11.4. To a solution of (2R,5R)-tert-butyl 5-((R)-1-(3-(benzyloxy)picolinamido)-2-ethoxy-2-oxoethyl)-1-methylpyrrolidine-2-carboxylate (11.3, 1.42 g, 2.85 mmol, 1 eq) in THF (15 mL) was added LiBH₄ (4 M, 1.07 mL, 1.5 eq). The mixture was stirred at 15° C. for 4 hrs. The reaction mixture was quenched by addition of 1M NaOH (7 mL) at 0° C., and then diluted with water 10 mL and extracted with ethyl acetate (10 mL×3). The combined organic layers were dried over Na₂SO₄, filtered and concentrated under reduced pressure to give a residue. The crude product was purified by prep-HPLC (column: Waters Xbridge BEH C18 250*70 mm*10 um; mobile phase: [water (NH₄HCO₃)-ACN]; B %: 30%-60%, 20 min) to give 0.9 g of 11.4 (34%) as a light yellow oil. (ES, m/z): [M+1]+456.4.

Synthesis of 11.5. To a solution (2R,5R)-tert-butyl 5-((R)-1-(3-(benzyloxy)picolinamido)-2-hydroxyethyl)-1-methylpyrrolidine-2-carboxylate (11.4, 840 mg, 1.84 mmol, 1 eq) in EtOH (15 mL) was added a catalytic amount of Pd/C (400 mg, 10% purity) and the flask was then purged and filled with an H₂ atmosphere at 15° C. under 15 psi. The reaction mixture was stirred for 16 hr at 15° C. The catalyst Pd/C was removed by filtration over celite with EtOH (15 mL×3) rinsing. The filtrate was concentrated to give 0.77 g of 11.5 (crude) as a light yellow oil. (ES, m/z): [M+1]⁺366.3.

Synthesis of 11.6. To a solution (2R,5R)-tert-butyl 5-((R)-2-hydroxy-1-(3-hydroxypicolinamido)ethyl)-1-methylpyrrolidine-2-carboxylate (133.5, 664 mg, 1.82 mmol, 1 eq) in THF (7 mL) was added Burgess reagent (649.53 mg, 2.73 mmol, 1.5 eq). The reaction mixture was stirred at 60° C. for 1 hr. The reaction mixture was concentrated under reduced pressure to give a residue. The crude product was purified by reversed-phase HPLC (column: Phenomenex luna C18 (250*70 mm, 10 um); mobile phase: [water (TFA)-ACN]; B %: 0%-35%, 25 min) to give 356 mg of 11.6 (51%) as a yellow oil. (ES, m/z): [M+1]⁺348.3.

Synthesis of I-11. To a solution of (2R, 5R)-tert-butyl 5-((R)-2-(3-hydroxypyridin-2-yl)-4,5-dihydrooxazol-4-yl)-1-methylpyrrolidine-2-carboxylate (11.6, 30 mg, 86.35 umol, 1 eq) in dichloromethane (0.5 mL) and TFA (4.5 mL) was added anisole (93.38 mg, 863.54 umol, 93.85 uL, 10 eq). The mixture was stirred at 15° C. for 12 hrs. The reaction mixture was filtered and concentrated under reduced pressure to give a residue. The crude product was purified by reversed-phase HPLC (column: Waters Xbridge BEH C18 100*30 mm*10 um; mobile phase: [water (NH₄HCO₃)-ACN]; B %: 1%-15%, 8 min) to give 8 mg of I-11 (22%, de value: 100%) as a white solid. (ES, m/z): [M+1]⁺292.1.

I-11: ¹H NMR (400 MHz, methanol-d₄) δ=7.55 (br s, 1H), 7.62-7.50 (m, 2H), 4.86-4.51 (m, 2H), 4.09 (t, J=3.2 Hz, 1H), 3.66 (d, J=6.4 Hz, 1H), 3.41-3.39 (m, 1H), 2.48 (s, 3H), 2.14-2.11 (m, 2H), 1.78-1.65 (m, 2H).

### Example 12. (2R,5S)-5-((S)-2-(2-hydroxyphenyl)-4,5-dihydrothiazol-4-yl)-1-methylpyrrolidine-2-carboxylic acid

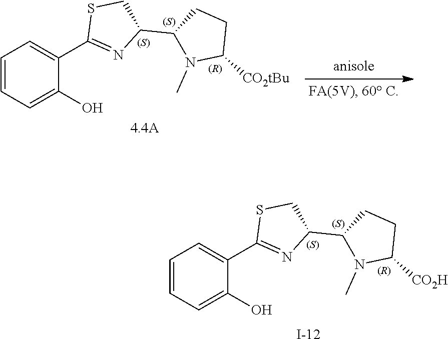

Synthesis of I-12. A mixture of tert-butyl(2R,5S)-5-[(4S)-2-(2-hydroxyphenyl)-4,5-dihydrothiazol-4-yl]-1-methyl-pyrrolidine-2-carboxylate (4.4 A, 500 mg, 1.38 mmol, 1 eq), anisole (1.49 g, 13.79 mmol, 1.50 mL, 10 eq) in formic acid (10 mL) was degassed and purged with N₂ 3 times, and then the mixture was stirred at 60° C. for 2 hrs under N₂ atmosphere. The reaction mixture concentrated under reduced pressure to give a residue. The residue was purified by prep-HPLC (column: 2_Phenomenex Gemini C18 75*40 mm*3 um; mobile phase: [H₂O (10 mM NH₄HCO₃)-ACN]; gradient: 10%-40% B over 8.0 min) to give 30.6 mg of I-12 (7.2%) as a green solid. (ES, m/z): [M+1]⁺307.0

I-12: ¹H NMR (400 MHz, methanol-d₄) δ=7.47-7.43 (m, 1H), 7.41-7.34 (m, 1H), 6.97-6.93 (m, 1H), 6.92-6.86 (m, 1H), 5.23 (dt, J=4.0, 9.4 Hz, 1H), 3.84 (dd, J=4.8, 9.0 Hz, 1H), 3.75 (ddd, J=3.6, 6.4, 9.8 Hz, 1H), 3.65 (dd, J=8.8, 11.4 Hz, 1H), 3.27 (t, J=10.8 Hz, 1H), 3.02 (s, 3H), 2.46-2.33 (m, 1H), 2.25-2.14 (m, 2H), 1.89-1.75 (m, 1H).

### Example 13. (2R,5R)—N-(2-(3-(but-3-yn-1-yl)-3H-diazirin-3-yl)ethyl)-5-((R)-2-(2-hydroxyphenyl)-4,5-dihydrothiazol-4-yl)-1-methylpyrrolidine-2-carboxamide

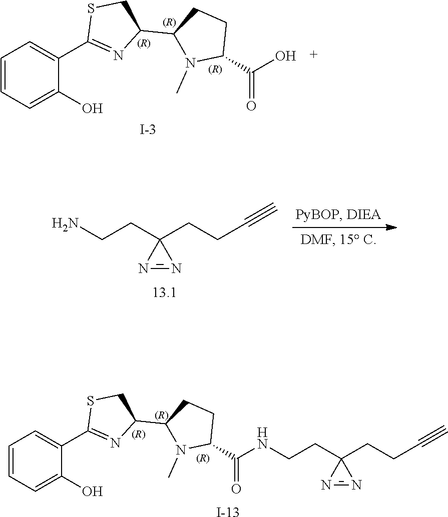

Synthesis of I-13. To a solution of 2-(3-(but-3-yn-1-yl)-3H-diazirin-3-yl) ethanamine (13.1, 8.96 mg, 65.28 umol, 2 eq), (2R,5R)-5-((R)-2-(2-hydroxyphenyl)-4,5-dihydrothiazol-4-yl)-1-methylpyrrolidine-2-carboxylic acid (I-3, 10.00 mg, 32.64 umol, 1 eq) in DMF (1 mL) was added PYBOP (26.16 mg, 50.27 umol, 1.54 eq) and DIEA (13.08 mg, 101.18 umol, 17.62 uL, 3.1 eq). The mixture was stirred at 15° C. for 16 hrs. The residue was purified by prep-HPLC (column: Phenomenex C18 80*40 mm*3 um; mobile phase: [water (NH₄HCO₃)-ACN]; B %: 30%-60%, 8 min) to give 8.97 mg of I-13 (64.58%, ee value: 89.8%) as a light yellow solid. (ES, m/z): [M+1]⁺426.2.

I-13: ¹H NMR (400 MHz, chloroform-d) δ=12.49 (s, 1H), 7.43-7.33 (m, 2H), 7.00 (d, J=8 Hz, 2H), 6.92-6.85 (m, 1H), 5.10 (dt, J=4, 13.2 Hz, 1H), 3.55-3.42 (m, 3H), 3.20-3.07 (m, 3H), 2.64 (s, 3H), 2.32-2.20 (m, 1H), 2.14-1.98 (m, 4H), 1.94-1.77 (m, 2H), 1.76-1.64 (m, 4H)

### Example 14. (2R,5S)—N-(2-(3-(but-3-yn-1-yl)-3H-diazirin-3-yl)ethyl)-5-((R)-2-(2-hydroxyphenyl)-4,5-dihydrothiazol-4-yl)-1-methylpyrrolidine-2-carboxamide

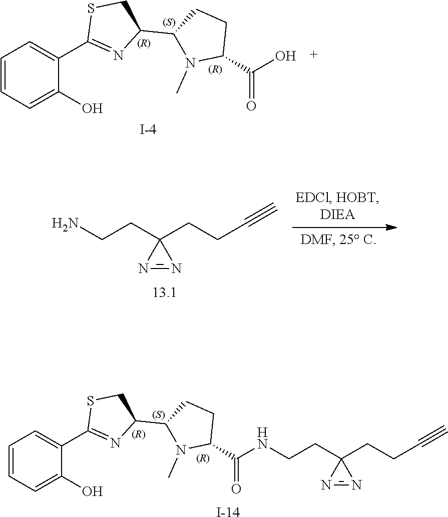

Synthesis of I-14. To a solution of (2R,5S)-5-((R)-2-(2-hydroxyphenyl)-4,5-dihydrothiazol-4-yl)-1-methylpyrrolidine-2-carboxylic acid (I-4, 70 mg, 228.47 umol, 1 eq) in DMF (0.5 mL) was added EDCI (52.56 mg, 274.17 umol, 1.2 eq), HOBt (37.05 mg, 274.17 umol, 1.2 eq), DIEA (88.58 mg, 685.42 umol, 119.39 uL, 3 eq) and 2-(3-(but-3-yn-1-yl)-3H-diazirin-3-yl) ethanamine (47.01 mg, 342.71 umol, 0.62 uL, 1.5 eq). The mixture was stirred at 25° C. for 3 hrs. The residue was purified by prep-HPLC (column: Waters Xbridge BEH C18 100*30 mm*10 um; mobile phase: [water (NH₄HCO₃)-ACN]; B %: 40%-60%, 8 mins) to give 32.44 mg of I-14 mixture (Stereochemical ratio: 5:94) as a light yellow gum. 32.44 mg of I-14 mixture (mixture of 2 peaks) was purified by SFC separation to give 19.99 mg (20.56%, ee value: 99.92%) of I-14 as a light yellow gum (ES, m/z): [M+1]⁺426.1.

Method 1: Instrument: Waters SFC 80 preparative SFC; Column: DAICEL CHIRALCEL OJ (250 mm*30 mm, 10 um); Mobile phase: A for CO₂ and B for IPA (0.1% NH₃H₂O); Gradient: B %=33% isocratic elution mode; Flow rate: 60 g/min; Wavelength: 220 nm; Column temperature: 40° C.; System back pressure: 100 bar.

I-14: ¹H NMR (400 MHz, methanol-d₄) δ=7.44 (br d, J=8.0 Hz, 1H), 7.39-7.29 (m, 1H), 6.97-6.82 (m, 2H), 5.04-4.92 (m, 1H), 3.58-3.48 (m, 1H), 3.43-3.34 (m, 1H), 3.23-3.16 (m, 1H), 3.15-3.05 (m, 2H), 3.04-2.95 (m, 1H), 2.54 (s, 3H), 2.28 (t, J=2.8 Hz, 1H), 2.23-2.12 (m, 1H), 2.04-1.93 (m, 3H), 1.89-1.73 (m, 2H), 1.64-1.52 (m, 4H).

### Example 15. (2R,5R)-5-((R)-2-(2-hydroxyphenyl)-4,5-dihydrothiazol-4-yl)-1-methyl-N-(16-oxo-20-((3aS,4S,6aR)-2-oxohexahydro-1H-thieno[3,4-d]imidazol-4-yl)-3,6,9,12-tetraoxa-15-azaicosyl) pyrrolidine-2-carboxamide

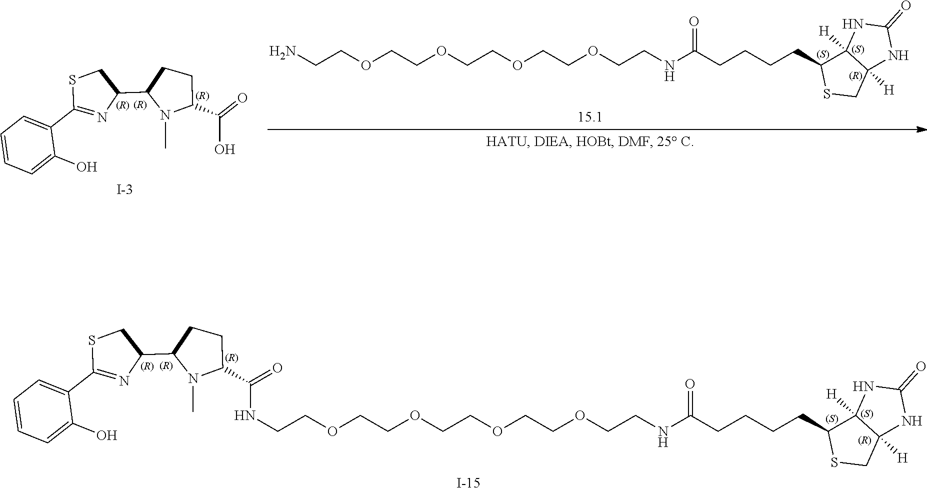

Synthesis of I-15. To a solution of (2R,5R)-5-((R)-2-(2-hydroxyphenyl)-4,5-dihydrothiazol-4-yl)-1-methylpyrrolidine-2-carboxylic acid (I-3, 20.00 mg, 65.28 μmol, 1 eq) in DMF (1.5 mL) was added HATU (24.82 mg, 65.28 μmol, 1 eq), HOBt (882.06 μg, 6.53 μmol, 0.1 eq) and DIEA (25.31 mg, 195.84 μmol, 34.11 μL, 3 eq). The mixture was stirred at 25° C. for 0.5 hr. Then N-(14-amino-3,6,9,12-tetraoxatetradecyl)-5-((3aS,4S,6aR)-2-oxohexahydro-1H-thieno[3,4-d]imidazol-4-yl) pentanamide (15.1, 30.20 mg, 65.28 μmol, 1 eq) was added dropwise at 25° C. The resulting mixture was stirred at 25° C. for 2.5 hrs. The residue was purified by prep-HPLC (column: Phenomenex Luna C18 100*30 mm*3 um; mobile phase: [H₂O (0.2% FA)-ACN]; gradient: 1%-40% B over 8.0 mins) to give 15 mg of I-15 (crude) as a white solid, which was further purified by prep-HPLC (column: Phenomenex luna C18 100*40 mm*3 um; mobile phase: [H₂O (0.2% FA)-ACN]; gradient: 10%-30% B over 8.0 mins) to give 11.60 mg of I-15 (9.4%, ee value: 95.54%) as a white solid. (ES, m/z): [M+1]⁺751.5

I-15: ¹H NMR (400 MHz, DMSO-d₆) δ=12.83 (s, 1H), 8.46 (s, 1H), 7.91 (br t, J=5.6 Hz, 1H), 7.83 (br t, J=5.6 Hz, 1H), 7.43-7.38 (m, 2H), 6.98-6.92 (m, 2H), 6.41 (s, 1H), 6.35 (s, 1H), 4.95 (dt, J=3.1, 9.3 Hz, 1H), 4.32-4.27 (m, 1H), 4.15-4.09 (m, 1H), 3.50 (br d, J=1.2 Hz, 12H), 3.27-3.04 (m, 10H), 2.81 (dd, J=5.2, 12.4 Hz, 1H), 2.59 (s, 1H), 2.38 (s, 3H), 2.13-2.02 (m, 3H), 1.92-1.80 (m, 1H), 1.65-1.40 (m, 7H), 1.38-1.20 (m, 3H

### Example 16. (2R,5S)-5-((R)-2-(2-hydroxyphenyl)-4,5-dihydrothiazol-4-yl)-1-methyl-N-(16-oxo-20-((3aS,4S,6aR)-2-oxohexahydro-1H-thieno[3,4-d]imidazol-4-yl)-3,6,9,12-tetraoxa-15-azaicosyl) pyrrolidine-2-carboxamide

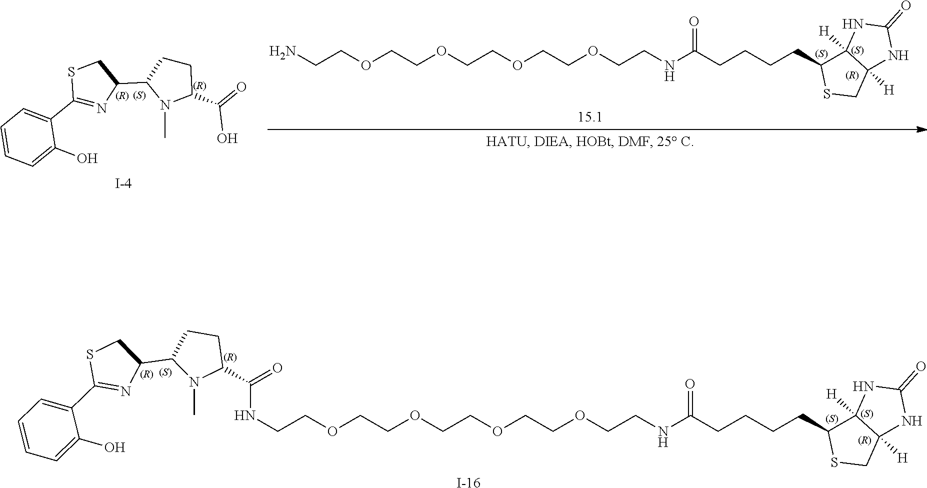

Synthesis of I-16. To a solution of (2R,5S)-5-((R)-2-(2-hydroxyphenyl)-4,5-dihydrothiazol-4-yl)-1-methylpyrrolidine-2-carboxylic acid (I-4, 15 mg, 48.96 μmol, 1 eq) in DMF (0.5 mL) was added DIEA (31.64 mg, 244.79 μmol, 42.64 μL, 5 eq) and the mixture was stirred at 25° C. for 2 hrs. Then HATU (40.95 mg, 107.71 μmol, 2.2 eq) and HOBt (661.53 μg, 4.90 μmol, 0.1 eq) was added and stirred for 0.5 hr. N-(14-amino-3,6,9,12-tetraoxatetradecyl)-5-((3aS,4S,6aR)-2-oxohexahydro-1H-thieno[3,4-d]imidazol-4-yl) pentanamide (15.1, 49.83 mg, 107.71 μmol, 2.2 eq) was added, and the mixture was stirred for another 0.5 hr. The mixture was purified by prep-HPLC (neutral condition; column: Waters Xbridge Prep OBD C18 150*40 mm*10 um; mobile phase: [H₂O (10 mM NH₄HCO₃)-ACN]; gradient: 20%-50% B over 8.0 min) to give 18.89 mg of I-16 (49.72%, ee value: 100%) as a white solid. (ES, m/z): [M+1]⁺751.3.

I-16: ¹H NMR (400 MHz, DMSO-d₆) δ=12.65 (s, 1H), 7.81 (br t, J=5.6 Hz, 1H), 7.67 (t, J=6.0 Hz, 1H), 7.45-7.38 (m, 2H), 7.00-6.91 (m, 2H), 6.44-6.32 (m, 2H), 4.94 (dt, J=5.6, 8.9 Hz, 1H), 4.30 (dd, J=5.2, 7.5 Hz, 1H), 4.15-4.09 (m, 1H), 3.55 (dd, J=9.0, 11.4 Hz, 1H), 3.49 (s, 11H), 3.43-3.34 (m, 6H), 3.26-3.14 (m, 4H), 3.13-3.05 (m, 2H), 3.00 (dd, J=6.8, 8.2 Hz, 1H), 2.81 (dd, J=5.1, 12.4 Hz, 1H), 2.57 (d, J=12.4 Hz, 1H), 2.41 (s, 2H), 2.44-2.38 (m, 1H), 2.09-1.97 (m, 3H), 1.85-1.73 (m, 1H), 1.73-1.72 (m, 1H), 1.72-1.62 (m, 1H), 1.72-1.38 (m, 4H), 1.37-1.21 (m, 2H)

### Example 17. 2-((R)-4-((2R,5R)-1-methyl-5-(prop-2-yn-1-ylcarbamoyl) pyrrolidin-2-yl)-4,5-dihydrothiazol-2-yl)phenyl sulfurofluoridate

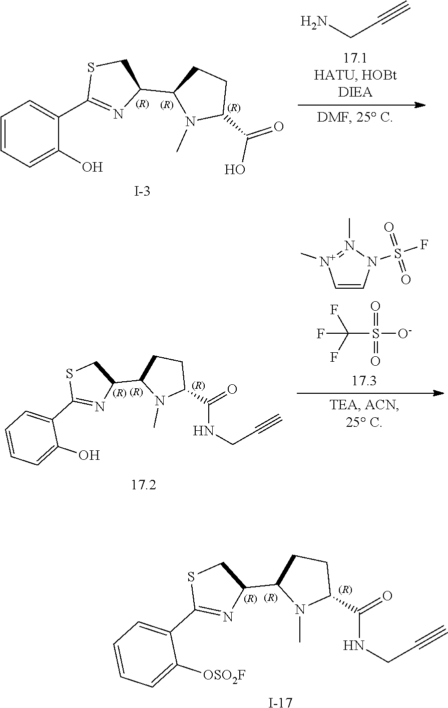

Synthesis of 17.2. To a solution of 5-[2-(2-hydroxyphenyl)-4,5-dihydrothiazol-4-yl]-1-methyl-pyrrolidine-2-carboxylic acid (1-3, 100 mg, 326.39 μmol, 1 eq) in DMF (2 mL) was added HATU (124.10 mg, 326.39 μmol, 1 eq), HOBt (48.51 mg, 359.03 umol, 1.1 eq) and DIEA (126.55 mg, 979.18 μmol, 170.56 μL, 3 eq). Then prop-2-yn-1-amine (17.1, 21.57 mg, 391.67 μmol, 25.08 μL, 1.2 eq) was added and the mixture was stirred at 25° C. for 2 hrs. The reaction mixture was filtered and concentrated under reduced pressure to give a residue. The residue was purified by prep-HPLC (column: Waters Xbridge Prep OBD C18 150*40 mm*10 um; mobile phase: [H₂O (10 mM NH₄HCO₃)-ACN]; gradient: 35%-65% B over 8.0 min) to give 120 mg of 17.2 (Crude) as a yellow oil. (ES, m/z): [M+1]⁺344.3.

Synthesis of I-17. To a solution of (2R)-5-[2-(2-hydroxyphenyl)-4,5-dihydrothiazol-4-yl]-1-methyl-N-prop-2-ynyl-pyrrolidine-2-carboxamide (17.2, 110 mg, 320.29 μmol, 1 eq) in ACN (1 mL) was added TEA (324.09 mg, 3.20 mmol, 445.80 μL, 10 eq). 2,3-dimethylimidazol-3-ium-1-sulfonyl fluoride; trifluoromethanesulfonate (17.3, 3.15 g, 9.61 mmol, 30 eq) was added and the mixture was stirred at 25° C. for 16 hrs. The reaction mixture was diluted with ethyl acetate 6 mL (2 ml×3) and extracted with H₂O 6 mL (2 ml×3). The combined organic layers were washed with brine and dried over Na₂SO₄. The reaction mixture was filtered and concentrated under reduced pressure to give a residue. The mixture was purified by prep-HPLC (column: Waters Xbridge Prep OBD C18 150*40 mm*10 um; mobile phase: [H₂O (10 mM NH₄HCO₃)-ACN]; gradient: 35%-65% B over 8.0 min) to give desired compound (80 mg, yield 57.51%, purity 91%) as a yellow oil, which was further separated by SFC separation to give 40.05 mg of I-17 (28%, ee value: 100%) as a yellow solid. (ES, m/z): [M+1]⁺426.2

Method 1: Instrument: Waters SFC 150AP preparative SFC; Column: DAICEL CHIRALPAK IH (250 mm*30 mm, 10 um); Mobile phase: A for CO₂ and B for IPA; Gradient: B %=35% isocratic elution mode; Flow rate: 70 g/min; Wavelength: 220 nm; Column temperature: 35° C.; System back pressure: 120 bar.

I-17: ¹H NMR (400 MHz, DMSO-d₆) δ=8.26 (br s, 1H), 7.87 (dd, J=1.2, 7.8 Hz, 1H), 7.77-7.69 (m, 2H), 7.67-7.62 (m, 1H), 4.82 (dt, J=3.2, 9.7 Hz, 1H), 3.85 (dd, J=1.6, 5.4 Hz, 2H), 3.60-3.47 (m, 3H), 3.29-3.25 (m, 1H), 3.07 (t, J=2.4 Hz, 1H), 2.36 (s, 3H), 2.14 (qd, J=8.8, 12.1 Hz, 1H), 1.98-1.87 (m, 1H), 1.69-1.54 (m, 2H)

### Example 18. 2—((R)-4-((2S,5R)-1-methyl-5-(prop-2-yn-1-ylcarbamoyl) pyrrolidin-2-yl)-4,5-dihydrothiazol-2-yl)phenyl sulfurofluoridate

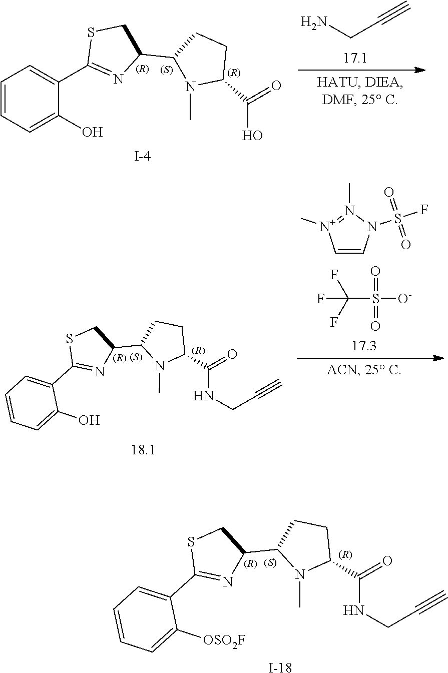

Synthesis of 18.1. To a solution of (2R,5S)-5-((R)-2-(2-hydroxyphenyl)-4,5-dihydrothiazol-4-yl)-1-methylpyrrolidine-2-carboxylic acid (I-4, 90 mg, 293.75 μmol, 1 eq) in DMF (0.5 mL) was added DIEA (189.83 mg, 1.47 mmol, 255.83 μL, 5 eq) and HATU (167.54 mg, 440.63 μmol, 1.5 eq) at 25° C. The mixture was stirred for 0.5 hr, then prop-2-yn-1-amine (17.1, 19.42 mg, 352.50 μmol, 22.58 μL, 1.2 eq) was added and the mixture was stirred for another 2.5 hrs. The mixture was purified by prep-HPLC (neutral condition: column: Waters Xbridge Prep OBD C18 150*40 mm*10 um; mobile phase: [H₂O (10 mM NH₄HCO₃)-ACN]; gradient: 30%-70% B over 8.0 min) to give 60 mg of 18.1 (59.47%, ee value: 88.36%) as a white solid. (ES, m/z): [M+1]⁺344.1

Synthesis of I-18. To a solution of (2R,5S)-5-((R)-2-(2-hydroxyphenyl)-4,5-dihydrothiazol-4-yl)-1-methyl-N-(prop-2-yn-1-yl) pyrrolidine-2-carboxamide (18.1, 50 mg, 145.58 μmol, 1 eq) in ACN (3 mL) was added TEA (147.32 mg, 1.46 mmol, 202.64 μL, 10 eq) and 1-(fluorosulfonyl)-2,3-dimethyl-1H-imidazol-3-ium trifluoromethanesulfonate (17.3, 1.43 g, 4.37 mmol, 30 eq). The mixture was stirred at 25° C. for 1 hr. The residue was purified by prep-HPLC (neutral condition; column: Waters Xbridge Prep OBD C18 150*40 mm*10 um; mobile phase: [H₂O (10 mM NH₄HCO₃)-ACN]; gradient: 35%-65% B over 8.0 min) to give desired compound (50 mg, yield 22.87%, purity 88%) as a yellow oil, which was further separated by SFC separating to give 13.65 mg of I-18 (21.99%, ee value: 100%) as a white solid. (ES, m/z): [M+1]⁺426.1

Method 1: neutral condition: Column: DAICEL CHIRALPAK IH (250 mm*30 mm, 10 um); Mobile phase: A for CO₂ and B for IPA; Gradient: B %=30% isocratic elution mode; Flow rate: 70 g/min; Wavelength: 220 nm; Column temperature: 35° C.; System back pressure: 120 bar.

I-18: ¹H NMR (400 MHz, chloroform-d) δ=7.84 (dd, J=1.6, 7.8 Hz, 1H), 7.61-7.54 (m, 2H), 7.54-7.42 (m, 2H), 4.84 (br d, J=6.8 Hz, 1H), 4.07-4.02 (m, 2H), 3.48 (br t, J=10.0 Hz, 1H), 3.32-3.17 (m, 3H), 2.56 (br s, 3H), 2.30-2.16 (m, 2H), 2.04-1.90 (m, 2H), 1.68 (br d, J=11.6 Hz, 1H)

### Example 19: (2R,5R)-5-((R)-2-(2-hydroxyphenyl)-4,5-dihydrooxazol-4-yl)-1-methylpyrrolidine-2-carboxylic acid (Compound I-85)

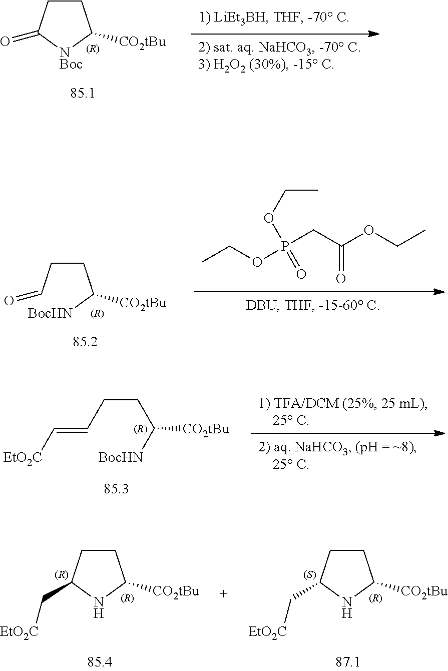

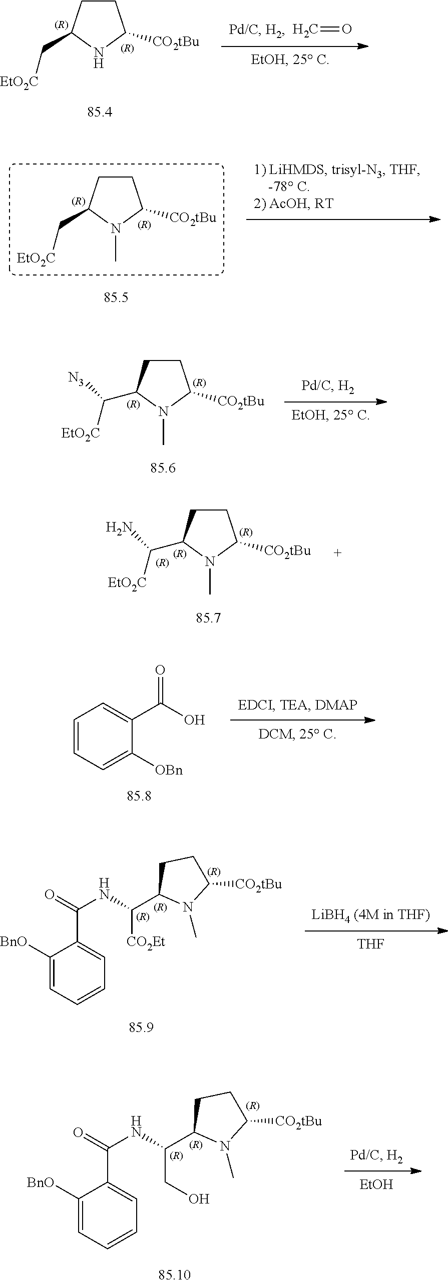

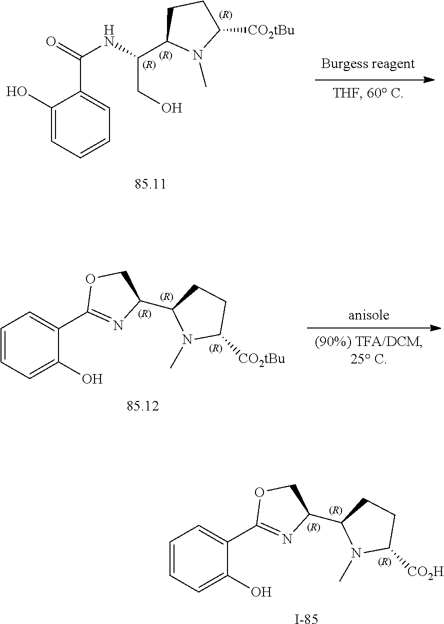

Synthesis of 85.2. A solution of di-tert-butyl(2R)-5-oxopyrrolidine-1,2-dicarboxylate (85.1, 40 g, 140.19 mmol, 1 eq) in THF (400 mL) was treated dropwise at −70° C. with LiEt₃BH (17.82 g, 168.22 mmol, 1.2 eq, 1 M in THF, 168.2 mL), then stirred at −70° C. for 30 min. The solution was carefully quenched with sat. aq. NaHCO₃ (160 mL). The mixture was warmed to −15° C. and H₂O₂ (94.40 g, 832.70 mmol, 80 mL, 30% purity, 5.94 eq) was added dropwise. After stirring at 0° C. for 30 min. The solution was decanted from a white solid residue and extracted with ethyl acetate (200 mL×2), washed with sat. aq. NaHCO₃ (100 mL), dried over Na₂SO₄, filtered, and evaporated under reduced pressure to give 44.2 g of 85.2 (crude) as an off-white oil. (ES, m/z): [M+1]⁺288.2.

Synthesis of 85.3. A suspension of DBU (38.15 g, 250.56 mmol, 37.77 mL, 1.8 eq) in THF (400 mL) was treated dropwise with ethyl 2-diethoxyphosphorylacetate (46.81 g, 208.80 mmol, 41.43 mL, 1.5 eq) and stirred for 2 h at 25° C. The reaction was cooled to −15° C. and a solution of the above (R)-tert-butyl 2-((tert-butoxycarbonyl)amino)-5-oxopentanoate (85.2, 40 g, 139.20 mmol, 1 eq) in THF (80 mL) was added dropwise. After the addition, the reaction was allowed to warm to 60° C. and stirred 16 h. The solution was cooled to 25° C. and poured carefully into sat. aq. NaHCO₃ (400 mL). The mixture was extracted with ethyl acetate (200 mL×2), dried over MgSO₄, filtered, and evaporated under reduced pressure to give the crude product. The residue was purified by column chromatography (SiO₂, petroleum ether/ethyl acetate=10/1 to 5/1) to give 34.6 g of 85.3 (70%) as a light yellow oil. (ES, m/z): [M+1]⁺358.2.

Synthesis of 85.4 and 87.1. To a solution of (R,E)-7-tert-butyl 1-ethyl 6-((tert-butoxycarbonyl)amino) hept-2-enedioate (85.3, 33.5 g, 93.72 mmol, 1 eq) in DCM (134 mL) was added TFA (51.59 g, 452.46 mmol, 33.50 mL, 4.83 eq). The mixture was stirred at 25° C. for 2 hr. Saturated aqueous NaHCO₃ (78.75 g, 937.34 mmol, 10 eq) was slowly added to above mixture and stirred at 25° C. for 12 h. The suspension was filtered through a pad of Celite and the filter cake was washed with DCM (50 ml×3). The combined filtrates were diluted with H₂O (100 mL) and extracted with DCM (50 mL×2). The combined organic layers were washed with brine, dried over Na₂SO₄, filtered and concentrated under reduced pressure to give a residue. The residue was purified by column chromatography repeat three times (SiO₂, 0etroleum ether/ethyl acetate=I/O to 1/1) to give 3.35 g of 85.4 (14%) as light yellow oil, 4.55 g of 87.1 (19%) as a light yellow oil and 3.35 g of mixture. The two isomers were later confirmed by NOE.

85.4 ¹H NMR (400 MHz, chloroform-d) 8=4.07 (q, J=7.1 Hz, 2H), 3.67-3.54 (m, 2H), 2.36 (dd, J=2.3, 6.8 Hz, 2H), 2.19-2.07 (m, 1H), 1.93-1.84 (m, 1H), 1.81-1.71 (m, 1H), 1.39 (s, 10H), 1.19 (t, J=7.2 Hz, 3H)

87.1 ¹H NMR (400 MHz, chloroform-d) 8=4.15 (q, J=7.1 Hz, 2H), 3.68 (dd, J=5.6, 8.7 Hz, 1H), 3.45 (br d, J=7.5 Hz, 1H), 2.61-2.42 (m, 2H), 2.13-2.04 (m, 1H), 2.01-1.86 (m, 3H), 1.46 (s, 9H), 1.39 (br d, J=12.0 Hz, 1H), 1.26 (t, J=7.1 Hz, 3H).

Synthesis of 85.5. To a solution of (2R,5R)-tert-butyl 5-(2-ethoxy-2-oxoethyl) pyrrolidine-2-carboxylate (85.4, 2.45 g, 9.52 mmol, 1 eq) in EtOH (24.5 mL) was treated with formaldehyde (15.45 g, 190.42 mmol, 14.18 mL, 37% purity, 20 eq) and 1 M aq. HCl until a pH=2 was obtained. A catalytic amount of Pd/C (0.245 g, 10% purity) was added and the flask was then purged and filled with an H₂ atmosphere at 25° C. under 15 Psi. Then the mixture was stirred at 25° C. for 16 h. The catalyst Pd/C was removed by filtration over Celite with EtOH (3×5 mL) rinsing. The filtrate was modified pH=7-8 with sat. aq. NaHCO₃, and then concentrated to removed EtOH and give a residue. The residue was extracted with ethyl acetate (2×20 mL). The combined organic layers were dried over anhydrous sodium sulfate, filtered and concentrated under reduced pressure to give a residue. The residue was purified by flash silica gel chromatography (ISCO®; 20 g SepaFlash® silica flash column, eluent of 0~22% ethyl acetate/petroleum ether gradient @ 36 mL/min) to give 2.37 g of 85.5 (92%) as a light yellow oil. (ES, m/z): [M+1]⁺272.2.

Synthesis of 85.6. To a solution of (2R,5R)-tert-butyl 5-(2-ethoxy-2-oxoethyl)-1-methylpyrrolidine-2-carboxylate (85.5, 1.5 g, 5.53 mmol, 1 eq) in dry THF (9.6 mL) was cooled to −78° C. and treated with LiHMDS (1 M, 12.16 mL, 2.2 eq) dropwise. The enolate was stirred over 1 h at −78° C. and treated with a precooled (−78° C.) solution of 2,4,6-triisopropylbenzenesulfonyl azide (3.42 g, 11.06 mmol, 2 eq) in THF (22.4 mL). After 10 min the reaction was quenched by adding AcOH (1.33 g, 22.11 mmol, 1.26 mL, 4 eq) and then the resulting mixture was stirred at 25° C. for 16 h. The reaction was concentrated, taken up in 10% aq. Na₂CO₃ (20 mL) and extracted with ethyl acetate (20 mL×2), the combined organic phase was dried over anhydrous sodium sulfate, filtered and concentrated in vacuum. The resulted residue was purified by flash silica gel chromatography (ISCO®; 40 g SepaFlash® silica flash column, eluent of 0~40% ethyl acetate/petroleum ether gradient @ 86 mL/min) to give 950 mg of 85.6 (55%) as a yellow oil. (ES, m/z): [M+1]⁺313.3.

Synthesis of 85.7. A solution of (2R,5R)-tert-butyl 5-((R)-1-azido-2-ethoxy-2-oxoethyl)-1-methylpyrrolidine-2-carboxylate (85.6, 950 mg, 3.04 mmol, 1 eq) in EtOH (10 mL) was treated with catalytic amount of Pd/C (250 mg, 10% purity) and placed under a H₂ atmosphere (balloon). The reaction was stirred at 25° C. for 16 h. The mixture was purged with a stream of N₂ and filtered through celite with EtOH rinsing. Evaporation of the filtrate to give 900 mg of 85.7 (88%) as a yellow oil. The crude product was used into the next step without further purification. (ES, m/z): [M+1]⁺ 287.1.

Synthesis of 85.9. A solution of (2R,5R)-tert-butyl 5-((R)-1-amino-2-ethoxy-2-oxoethyl)-1-methylpyrrolidine-2-carboxylate (85.7, 900 mg, 3.14 mmol, 1 eq) which was taken up directly in DCM (9 mL) and treated with 2-(benzyloxy)benzoic acid (85.8, 860.79 mg, 3.77 mmol, 1.2 eq), EDCI (753.11 mg, 3.93 mmol, 1.25 eq), TEA (397.52 mg, 3.93 mmol, 546.80 μL, 1.25 eq), and a catalytic amount of DMAP (38.40 mg, 314.28 umol, 0.1 eq) and stirred for 16 h at 25° C. Then the mixture was dried in vacuum and purified by flash silica gel chromatography (ISCO®; 20 g SepaFlash® silica flash column, eluent of 0~15% ethyl acetate/petroleum ether gradient @ 80 mL/min) to give 1.1 g of 85.9 (70%, de value: 83.7%) as a sticky light yellow oil. (ES, m/z): [M+1]⁺497.3.

Synthesis of 85.10. To a solution of (2R,5R)-tert-butyl 5-((R)-1-(2-(benzyloxy)benzamido)-2-ethoxy-2-oxoethyl)-1-methylpyrrolidine-2-carboxylate (85.9, 900 mg, 1.81 mmol, 1 eq) in THF (9 mL) was treated with LiBH₄ (4 M, 679.63 μL, 1.5 eq) and stirred for 4 h at 25° C. And another LiBH₄ (4 M, 226.54 μL, 0.5 eq) was added to the reaction and stirred for 1 h at 25° C. The reaction was quenched by addition of 1M NaOH (2 mL), stirred for an additional 10 min, and then diluted with water (5 mL) and with ethyl acetate (5 mL×3). The combined organic layers were dried over Na₂SO₄, filtered, and evaporated filtered and concentrated in vacuum. The residue was purified by prep-TLC (petroleum ether:ethyl acetate=1:1) to give 409 mg of 85.10 (50%) as a colorless oil. (ES, m/z): [M+1]⁺455.3.

Synthesis of 85.11. A mixture of (2R,5R)-tert-butyl 5-((R)-1-(2-(benzyloxy)benzamido)-2-hydroxyethyl)-1-methylpyrrolidine-2-carboxylate (85.10, 500 mg, 1.10 mmol, 1 eq) in EtOH (5 mL) was degassed and purged with N₂ for 3 times, and treated with a catalytic amount of Pd/C (200 mg, 10% purity) and placed under H₂ atmosphere (balloon). The reaction was stirred at 25° C. for 12 h. The mixture was purged with a stream of Ar, filtered over celite with ethyl acetate rinsing and evaporated to give the crude phenol intermediate 345 mg of 85.11 (86%) as a sticky yellow oil. (ES, m/z): [M+1]⁺365.3.

Synthesis of 85.12. To a solution of (2R,5R)-tert-butyl 5-((R)-2-hydroxy-1-(2-hydroxybenzamido)ethyl)-1-methylpyrrolidine-2-carboxylate (85.11, 315 mg, 864.35 umol, 1 eq) in THF (3 mL) was added Burgess reagent (308.97 mg, 1.30 mmol, 1.5 eq) and stirred at 60° C. for 1 h. The residue was purified by flash silica gel chromatography (ISCO®; 25 g SepaFlash® silica flash column, eluent of 0~10% ethyl acetate/petroleum ether gradient @ 86 mL/min) to give 200 mg of 85.12 (66%, de value: 85.6%) as a yellow solid. (ES, m/z): [M+1]⁺347.2.

Synthesis of I-85. To a solution of (2R,5R)-tert-butyl 5-((R)-2-(2-hydroxyphenyl)-4,5-dihydrooxazol-4-yl)-1-methylpyrrolidine-2-carboxylate (85.12, 20 mg, 57.73 μmol, 1 eq) in a mixture of TFA (0.9 mL) and DCM (0.1 mL) was added anisole (62.43 mg, 577.33 μmol, 62.75 μL, 10 eq) at 25° C. The reaction mixture was stirred 6 h at 25° C. The reaction mixture was concentrated under reduced pressure to give a residue. The residue was purified by prep-HPLC (column: Phenomenex C18 80*40 mm*3 um; mobile phase: [water (NH₄HCO₃)-ACN]; B %: 5%-35%, 8 min) to give 9.5 mg of I-85 (56%, de value: 100%) as a light yellow solid. (ES, m/z): [M+1]⁺291.1. ¹H NMR (400 MHz, chloroform-d) 8=7.66 (dd, J=1.1, 7.9 Hz, 1H), 7.46-7.38 (m, 1H), 7.02 (d, J=8.5 Hz, 1H), 6.90 (t, J=7.6 Hz, 1H), 4.88-4.75 (m, 1H), 4.58 (t, J=9.4 Hz, 1H), 4.17 (t, J=8.2 Hz, 1H), 3.77 (dt, J=4.4, 9.8 Hz, 2H), 2.82 (s, 3H), 2.33 (qd, J=8.7, 12.7 Hz, 1H), 2.22-2.09 (m, 1H), 2.07-1.95 (m, 1H), 1.83 (ddd, J=4.4, 8.7, 13.0 Hz, 1H).

### Example 20: (2R,5R)-5-((R)-2-(2-hydroxyphenyl)-4,5-dihydrothiazol-4-yl)-1-methylpyrrolidine-2-carboxylic acid (Compound I-86)

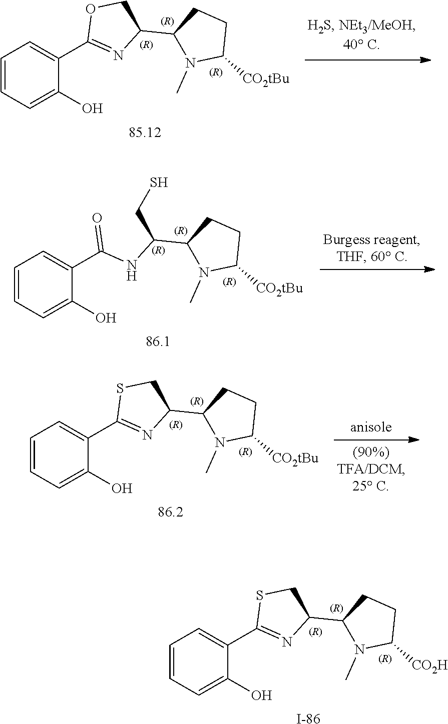

Synthesis of 86.1. To a solution of (2R,5R)-tert-butyl 5-((R)-2-(2-hydroxyphenyl)-4,5-dihydrooxazol-4-yl)-1-methylpyrrolidine-2-carboxylate (85.12, 200 mg, 577.33 μmol, 1 eq) was dissolved in TEA (6 mL) and MeOH (6 mL) at 25° C. in a pressure flask. H₂S gas was then bubbled through the stirring solution for 20 min, after which the pressure flask was closed and warmed to 40° C. The reaction was monitored periodically by LCMS and stirred at 40° C. for 7 days. The mixture was concentrated to give 220 mg of 86.1 (crude) as a yellow solid. The crude product was used into the next step without further purification. (ES, m/z): [M+1]⁺381.3.

Synthesis of 86.2. To a solution of (2R,5R)-tert-butyl 5-((R)-1-(2-hydroxybenzamido)-2-mercaptoethyl)-1-methylpyrrolidine-2-carboxylate (86.1, 220 mg, 578.19 μmol, 1 eq) in THF (3 mL) was added Burgess Reagent (206.68 mg, 867.29 μmol, 1.5 eq) under N₂. The mixture was stirred at 60° C. for 2 hours. The residue was purified by prep-TLC (SiO₂, petroleum ether/ethyl acetate=5/1) to give 50 mg of 86.2 (24%, de value: 100%) as a yellow solid. (ES, m/z): [M+1]⁺363.2.

Synthesis of I-86. To solution of (2R,5R)-tert-butyl 5-((R)-2-(2-hydroxyphenyl)-4,5-dihydrothiazol-4-yl)-1-methylpyrrolidine-2-carboxylate (50 mg, 137.94 umol, 1 eq) in TFA (0.9 mL) and DCM (0.1 mL) was added anisole (149.16 mg, 1.38 mmol, 149.91 μL, 10 eq). The reaction mixture was stirred for 16 h at 25° C. LCMS showed the reactant was consumed completely and desired MS was detected. The reaction mixture was concentrated under reduced pressure to give a residue. The residue was purified by prep-HPLC (column: Phenomenex C18 80*40 mm*3 um; mobile phase: [water (NH₄HCO₃)-ACN]; B %: 5%-35%, 8 min) to give 20.76 mg of I-86 (49%, de value: 100%) as a yellow solid. (ES, m/z): [M+1]⁺307.1. ¹H NMR (400 MHz, methanol-d₄) δ=7.48 (dd, J=1.5, 7.9 Hz, 1H), 7.40 (dt, J=1.5, 7.8 Hz, 1H), 6.99-6.88 (m, 2H), 5.18 (dt, J=3.4, 9.3 Hz, 1H), 4.04-3.86 (m, 2H), 3.64 (dd, J=9.1, 11.3 Hz, 1H), 3.23 (dd, J=9.5, 11.3 Hz, 1H), 2.96 (s, 3H), 2.38-2.21 (m, 2H), 2.15-1.90 (m, 2H).

### Example 21: (2R,5S)-5-((R)-2-(2-hydroxyphenyl)-4,5-dihydrooxazol-4-yl)-1-methylpyrrolidine-2-carboxylic acid (Compound I-87)

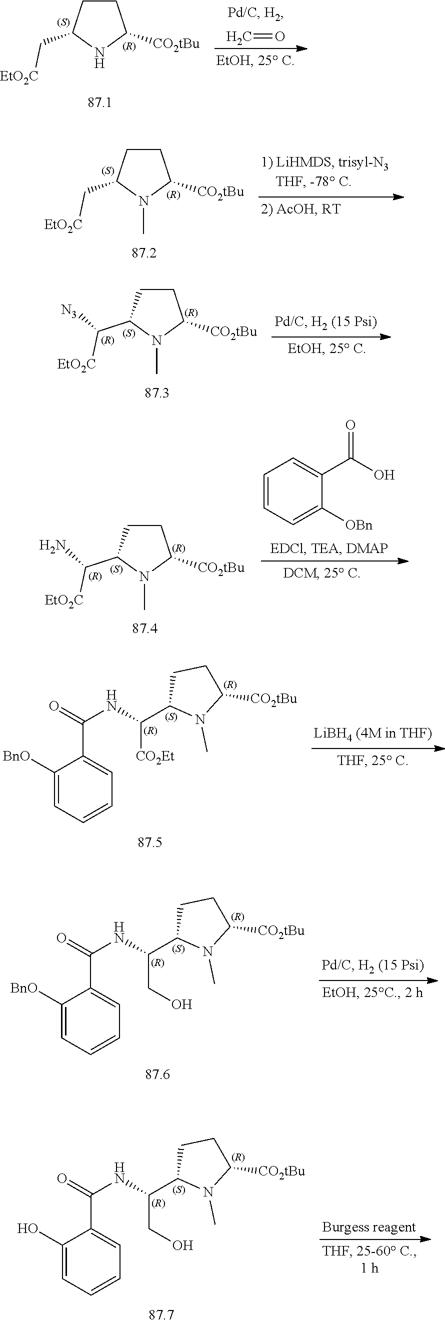

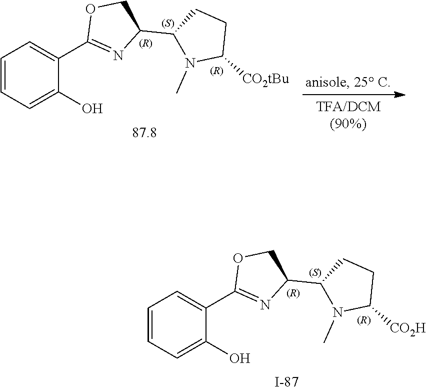

Synthesis of 87.2. The tert-butyl(2R,5S)-5-(2-ethoxy-2-oxo-ethyl) pyrrolidine-2-carboxylate (87.1, 4.2 g, 16.32 mmol, 1 eq) in EtOH (42 mL) was treated with formaldehyde (26.49 g, 326.44 mmol, 24.31 mL, 37% purity, 20 eq) and added 1 M aq. HCl until pH=2. A catalytic amount of Pd/C (420 mg, 10% purity) was added and the flask was then purged and placed under H₂ (15 Psi) atmosphere at 25° C. for 16 h. The catalyst Pd/C was removed by filtration over celite with EtOH (5 mL×3) rinsing. The filtrate was modified pH=7-8 with sat. aq. NaHCO₃, and then concentrated to remove EtOH. The residue was extracted with ethyl acetate (10 mL×2), drying over Na₂SO₄, filtration, and concentration under reduced pressure gave a crude. The residue was purified by column chromatography (SiO₂, petroleum ether/ethyl acetate=10/1 to 5/1) to obtain 4.23 g of 87.2 (96%) as a light yellow oil. (ES, m/z): [M+1]⁺272.2.

Synthesis of 87.3. A solution of tert-butyl(2R,5S)-5-(2-ethoxy-2-oxo-ethyl)-1-methyl-pyrrolidine-2-carboxylate (87.2, 2 g, 7.37 mmol, 1 eq) in dry THF (14 mL) was cooled to −78° C. and treated with LiHMDS (1 M, 16.22 mL, 2.2 eq) dropwise. The enolate was allowed to form over 1 h at −78° C. and treated with a precooled (−78° C.) solution of N-diazo-2,4,6-triisopropyl-benzenesulfonamide (4.56 g, 14.74 mmol, 2 eq) in THF (28 mL). After 10 min the reaction was quenched by adding glacial AcOH (1.77 g, 29.48 mmol, 1.69 mL, 4 eq) and then warmed to 25° C. and stirred for 16 h. The reaction was concentrated, taken up in 10% aq. Na₂CO₃ (20 mL), and extracted with ethyl acetate (10 mL×2), then the organic extracts were dried over Na₂SO₄, filtered, and concentrated in vacuum. The residue was purified by column chromatography (SiO₂, Petroleum ether/Ethyl acetate=10/1 to 5/1) to give 1.16 g of 87.3 (49%) as a light yellow oil. (ES, m/z): [M+1]⁺312.2.

Synthesis of 87.4. To (2R,5S)-tert-butyl 5-((R)-1-azido-2-ethoxy-2-oxoethyl)-1-methylpyrrolidine-2-carboxylate (87.3, 1.1 g, 3.69 mmol, 1 eq) in EtOH (11 mL) was added a catalytic amount of Pd/C (110 mg, 10% purity) and the flask was then purged and filled with an H₂ atmosphere at 25° C. under 15 Psi. The reaction mixture was stirred 2 h at 25° C. The catalyst Pd/C was removed by filtration over celite with EtOH (5 mL*3) rinsing. The filtrate was concentrated under reduced pressure to give 1.06 g of 87.4 (crude) as a light yellow oil. (ES, m/z): [M+1]⁺287.2.

Synthesis of 87.5. To a solution of (2R,5S)-tert-butyl 5-((R)-1-amino-2-ethoxy-2-oxoethyl)-1-methylpyrrolidine-2-carboxylate (87.4, 1.06 g, 3.70 mmol, 1 eq) and 2-benzyloxybenzoic acid (1.01 g, 4.44 mmol, 1.2 eq) in DCM (10 mL) was added EDCI (851.51 mg, 4.44 mmol, 1.2 eq), TEA (449.47 mg, 4.44 mmol, 618.26 μL, 1.2 eq) and DMAP (45.22 mg, 370.16 μmol, 0.1 eq). The mixture was stirred at 25° C. for 12 hr. The mixture was concentrated to afford the crude product and purified by column chromatography (SiO₂, petroleum ether/ethyl acetate=10/1 to 1/1) to give 1.52 g of 87.5 (80%, de value: 100%) as a light yellow oil. (ES, m/z): [M+1]⁺497.3

Synthesis of 87.6. A solution of (2R,5S)-tert-butyl 5-((R)-1-(2-(benzyloxy)benzamido)-2-ethoxy-2-oxoethyl)-1-methylpyrrolidine-2-carboxylate (87.5, 1.35 g, 2.72 mmol, 1 eq) in THF (14 mL) was treated with LiBH₄ (4 M in THF, 1.02 mL, 1.5 eq) at 0° C. and stirred for 4 h at 25° C. The reaction was quenched by addition of 1M NaOH (6 mL), stirred for an additional 10 min, then diluted with water (10 mL) and extracted with ethyl acetate (10 mL×2). The combined organic layers were dried over Na₂SO₄, filtered, and evaporated and the crude residue was purified by chromatography. The residue was purified by column chromatography (SiO₂, petroleum ether/ethyl acetate=10/1 to 5/1) to give 0.85 g of 87.6 (67%) as a light yellow oil. (ES, m/z): [M+1]⁺455.3

Synthesis of 87.7. To (2R,5S)-tert-butyl 5-((R)-1-(2-(benzyloxy)benzamido)-2-hydroxyethyl)-1-methylpyrrolidine-2-carboxylate (87.6, 850 mg, 1.87 mmol, 1 eq) in EtOH (8.5 mL) was added a catalytic amount of Pd/C (85 mg, 10% purity) and the flask was then purged and filled with an H₂ atmosphere at 25° C. under 15 Psi. The reaction mixture was stirred 2 h at 25° C. The catalyst Pd/C was removed by filtration over celite with EtOH (5 mL×3) rinsing. The filtrate was concentrated to give a residue. The residue was purified by prep-TLC (SiO₂, petroleum ether/ethyl acetate=1/2) to give 440 mg of 87.7 (64%, de value: 95.1%) as a light yellow oil. (ES, m/z): [M+1]⁺365.2.

Synthesis of 87.8. To (2R,5S)-tert-butyl 5-((R)-2-hydroxy-1-(2-hydroxybenzamido)ethyl)-1-methylpyrrolidine-2-carboxylate (87.7, 390 mg, 1.07 mmol, 1 eq) in THF (6 mL) was added Burgess Reagent (382.54 mg, 1.61 mmol, 1.5 eq). The reaction mixture was stirred 0.5 h at 25° C. and 0.5 h at 60° C. The reaction mixture was concentrated under reduced pressure to give a residue. The residue was purified by prep-TLC (SiO₂, petroleum ether/ethyl acetate=2/1) to give 210 mg of 87.8 (57%, de value: 99.9%) as a light yellow oil. (ES, m/z): [M+1]⁺347.2.

Synthesis of I-87. To (2R,5S)-tert-butyl 5-((R)-2-(2-hydroxyphenyl)-4,5-dihydrooxazol-4-yl)-1-methylpyrrolidine-2-carboxylate (87.8, 40 mg, 115.47 μmol, 1 eq) in TFA (1.35 mL) and DCM (0.15 mL) was added anisole (124.87 mg, 1.15 mmol, 125.49 μL, 10 eq). The reaction mixture was stirred 6 h at 25° C. The reaction mixture was concentrated under reduced pressure to give a residue. The residue was purified by prep-HPLC (column: Phenomenex C18 80*40 mm*3 um; mobile phase: [water (NH₄HCO₃)-ACN]; B %: 5%-25%, 8 min) to give 28.5 mg of I-87 (84%, de value: 100%) as a light yellow solid. (ES, m/z): [M+1]⁺291.1. ¹H NMR (400 MHz, methanol-d₄) δ=7.65 (dd, J=1.5, 7.9 Hz, 1H), 7.44-7.33 (m, 1H), 7.00-6.82 (m, 2H), 4.76-4.62 (m, 1H), 4.55-4.37 (m, 2H), 3.19-3.09 (m, 1H), 2.95 (br d, J=2.3 Hz, 1H), 2.57 (s, 3H), 2.13 (qd, J=5.7, 11.0 Hz, 1H), 1.97-1.79 (m, 2H), 1.64-1.46 (m, 1H).

### Example 22: (2R,5S)-5-((R)-2-(2-hydroxyphenyl)-4,5-dihydrothiazol-4-yl)-1-methylpyrrolidine-2-carboxylic acid (Compound I-88)

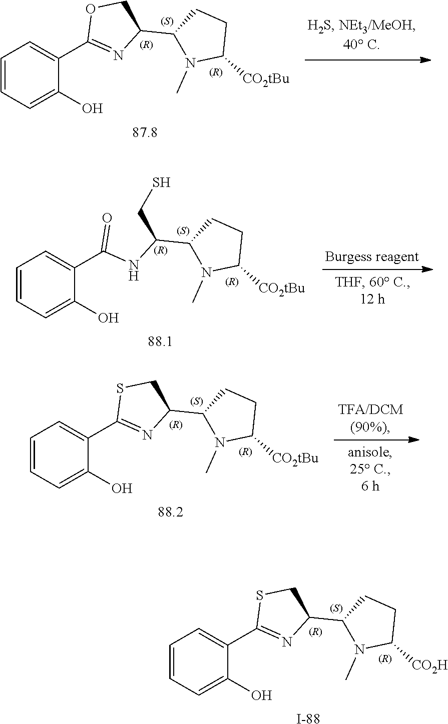

Synthesis of 88.1. A solution of (2R,5S)-tert-butyl 5-((R)-2-(2-hydroxyphenyl)-4,5-dihydrooxazol-4-yl)-1-methylpyrrolidine-2-carboxylate (87.8, 150 mg, 433.00 μmol, 1 eq) was dissolved in MeOH (2.5 mL) and TEA (2.5 mL) at 25° C. in a pressure flask. H₂S gas was then bubbled through the stirring solution for 20 min, after which the pressure flask was closed and warmed to 40° C. The reaction was monitored periodically by LCMS and stirred at 40° C. for 168 h. The reaction was then purged by bubbling a stream of Ar through the solution for 15 min. Concentration and drying under vacuum gave 165 mg of 88.1 (crude) as a yellow solid, which was used directly in the subsequent cyclodehydration. (ES, m/z): [M+1]⁺381.2.

Synthesis of 88.2. To (2R,5S)-tert-butyl 5-((R)-1-(2-hydroxybenzamido)-2-mercaptoethyl)-1-methylpyrrolidine-2-carboxylate (88.1, 165 mg, 433.64 μmol, 1 eq) in THF (6 mL) was added Burgess Reagent (155.01 mg, 650.46 μmol, 1.5 eq). The reaction mixture was stirred 12 h at 60° C. The reaction mixture was concentrated under reduced pressure to give a residue. The residue was purified by prep-TLC (SiO₂, petroleum ether/ethyl acetate=3/1) to give 66 mg of 88.2 (39%, de value: 100%) as a light yellow oil. (ES, m/z): [M+1]⁺363.2.

Synthesis of I-88. The (2R,5S)-tert-butyl 5-((R)-2-(2-hydroxyphenyl)-4,5-dihydrothiazol-4-yl)-1-methylpyrrolidine-2-carboxylate (88.2, 60 mg, 165.52 μmol, 1 eq) in TFA (1.35 mL) and DCM (0.15 mL) was added anisole (179.00 mg, 1.66 mmol, 179.90 μL, 10 eq). The reaction mixture was stirred 6 h at 25° C. The reaction mixture was concentrated under reduced pressure to give a residue. The residue was purified by prep-HPLC (column: Phenomenex C18 80*40 mm*3 um; mobile phase: [water (NH₄HCO₃)-ACN]; B %: 5%-35%, 8 min) to give 30.66 mg of I-88 (60%, de value: 100%) as a light green solid. (ES, m/z): [M+1]⁺307.1. ¹H NMR (400 MHz, methanol-d₄) δ=7.47 (br d, J=7.9 Hz, 1H), 7.40 (br t, J=7.8 Hz, 1H), 7.00-6.89 (m, 2H), 5.09 (q, J=8.4 Hz, 1H), 3.69 (br s, 1H), 3.63-3.44 (m, 2H), 3.27 (s, 1H), 2.88 (s, 3H), 2.39-2.27 (m, 1H), 2.24-2.12 (m, 2H), 1.84 (qd, J=8.8, 13.3 Hz, 1H)

### Example 23: TLR7/8 Stimulated Interferon Response Element and NFκB Dual Reporter Gene Assay in THP-1 Cells

Compounds described herein are added to the human leukemic monocyte-like cell line THP-1 for bioactivity assessment. THP1-Dual cells (InvivoGen, San Diego, CA) feature the Lucia gene, a secreted luciferase reporter gene, under the control of an ISG54 minimal promoter in conjunction with five IFN-stimulated response elements. THP1-Dual cells also express a secreted embryonic alkaline phosphatase (SEAP) reporter gene driven by an IFN-β minimal promoter fused to five copies of the NF-κB consensus transcriptional response element and three copies of the c-Rel binding site. As a result, THP1-Dual cells allow the simultaneous study of the NF-κB pathway, by monitoring the activity of SEAP, and the IRF pathway, by assessing the activity of Lucia luciferase.

THP-1 dual reporter cells were grown in RPMI 1640 culture medium with 2 mM L-glutamine, 25 mM HEPES, 10% heat-inactivated fetal bovine serum, 100 μg/ml Normocin antibiotic (InvivoGen), Pen-Strep antibiotic (100 U/ml-100 μg/ml, InvivoGen). On the day of the assay, cells were seeded in a 384 flat-bottom well plate at 25,000 cells/well and incubated for 2 hours. Cells were then incubated with 10 μM of the compounds described herein or with 1 μM of IKK-16 (Selleck), a potent inhibitor of IκB kinases (IKKs) or with DMSO (vehicle control) for 2 hours, followed by the addition of the synthetic TLR7/8 agonist R848 at 1 μM and incubation for 16 hours. After incubation was concluded, the cultures supernatants were collected and IRF related activity was assessed by measurement of the Lucia luciferase, using Quanti-Luc assay (InvivoGen). NF-κB pathway activity was assessed by quantifying SEAP using Quanti-Blue assay (InvivoGen).

Compounds described herein, when tested at 10 μM, show a range of inhibition of both the interferon response element reporter (Table 1) and the NFκB reporter (Table 2) stimulated by R848. Compounds I-3, I-4, I-7 and I-8 inhibit the IRF reporter ≥50% at 10 μM. Compounds I-4 and I-8 inhibit the NFκB reporter ≥50% at 10 μM.

TABLE 1

| | R848-driven IRF reporter |
|---|---|
| Compound | gene assay (Percent |
| Number | inhibition at 10 μM) |
| I-1 | 20 |
| I-2 | 31 |
| I-3 | 64 |
| I-4 | 65 |
| I-5 | 17 |
| I-6 | #N/A |
| I-7 | 56 |
| I-8 | 70 |
| I-9 | #N/A |
| I-10 | 3 |
| I-11 | 5 |
| I-12 | #N/A |
| I-13 | #N/A |
| I-14 | #N/A |
| I-15 | #N/A |
| I-16 | #N/A |
| I-17 | #N/A |
| I-18 | #N/A |

TABLE 2

| | R848-driven NFκB reporter |
|---|---|
| Compound | gene assay (Percent |
| Number | inhibition at 10 μM) |
| I-1 | 10 |
| I-2 | 5 |
| I-3 | 40 |
| I-4 | 57 |
| I-5 | 27 |
| I-6 | #N/A |
| I-7 | 28 |
| I-8 | 50 |
| I-9 | #N/A |
| I-10 | 14 |
| I-11 | 6 |
| I-12 | #N/A |
| I-13 | #N/A |
| I-14 | #N/A |
| I-15 | #N/A |
| I-16 | #N/A |
| I-17 | #N/A |
| I-18 | #N/A |

### Example 24: TLR4 Stimulated Pro-Inflammatory Cytokine Release in Primary Human Monocyte-Derived Induced Dendritic Cells (DC)

Monocyte derived DC (MoDC) were differentiated from healthy human donor derived CD14+ monocytes. Commercially available freshly prepared Leukopaks were obtained from healthy blood donor and kept refrigerated on ice before spin centrifuged and the pelleted cells are rinsed and resuspended with MACS buffer (Miltenyi Biotec). Next, CD14+ monocytes were purified using MultiMACS™ Cell24 Separator-Plus instrumentation and reagents according to the manufacturer (Miltenyi Biotec) instructions. The resulting purified CD14+ cell fraction was washed, enumerated, and subsequently cryopreserved for later use. At the experiment first day cryopreserved CD14+ cells were thawed, rinsed with culture media, and then resuspended in culture media principally composed of RPMI (Invitrogen) supplemented with 10% v/v heat inactivated FBS. For generation of MoDC, these CD14+ cells were enumerated and seeded into 75 cm² tissue culture flasks in 30 mL of culture media supplemented with GM-CSF (40-100 ng/ml) and IL-4 (25 ng/ml). Every other day, half of the culture media was replaced with fresh media (containing the same cytokine supplementation) and after 6-7 days of culture, fully differentiated and mature MoDC were obtained as confirmed by conventional Immuno-phenotype markers (levels of expression or absence of surface CD80, CD83, CD86, CD209, HLA-DR, CD14 & CD64) that were verified by flow-cytometry. Fully differentiated (mature) MoDCs, were seeded into 96 flat-well plates and incubated with various concentrations of the compounds described herein, or with control solution (DMSO), or with the pharmacologic inhibitors Cyclosporin-A (CsA) or BAY 61-3606 (which is a known inhibitor of Syk kinase) for 6 hours. Next, the cells were stimulated with Toll-like receptor agonists R848 (TLR7/8) or LPS (TLR4), and further incubated for 18-20 hours. After incubation concluded, culture supernatants were collected, and the levels of inflammatory cytokines and chemokines were measured in a triplicate setting for each condition by Luminex® multiplex assay.

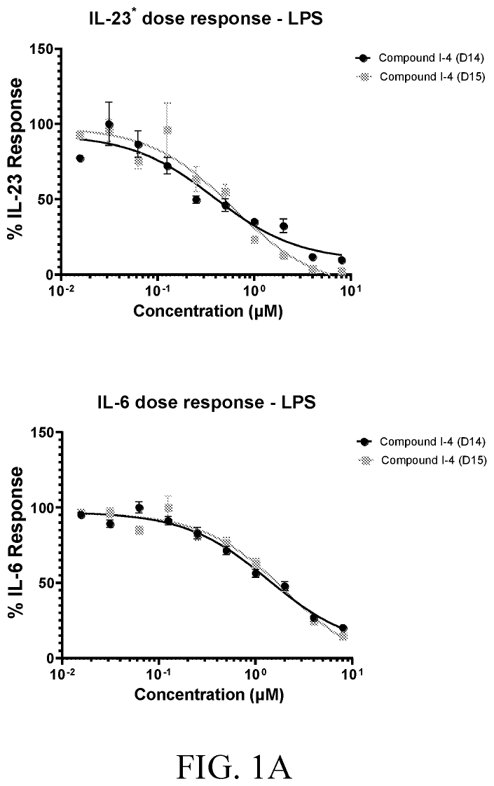

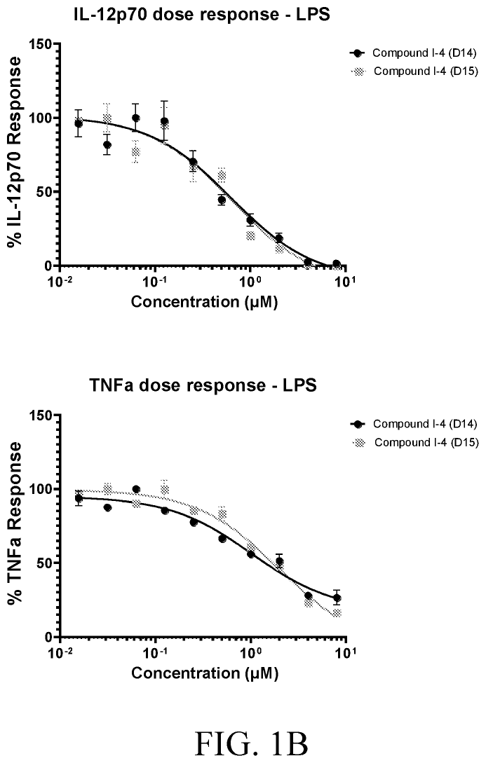

Compound I-4 inhibits several pro-inflammatory cytokines in LPS-stimulated MoDC from healthy human donors 14 (D14) and 15 (D15). Compound I-4 causes a statistically significant decrease in IL-23, IL-12p70, IL-6 and TNFα (FIGS. 1A and 1B).

### Example 25: LPS Induced Septic Shock Model in Mice In Vivo

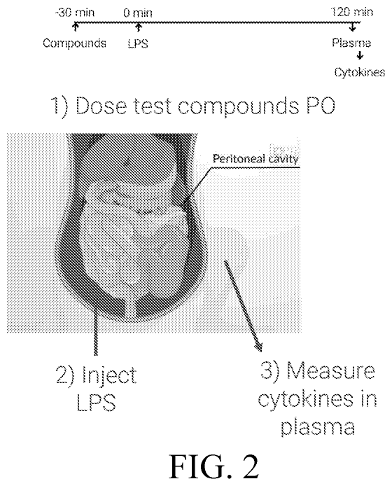

In the LPS induced septic shock model in mice, the TLR4 agonist LPS is injected into the peritoneal cavity of mice, inducing production of pro-inflammatory cytokines that can be measured in blood two hours later (FIG. 2).

All the procedures related to animal handling, care and the treatment in the study were performed according to the guidelines approved by the Institutional Animal Care and Use Committee (IACUC) of WuXi AppTec following the guidance of the Association for Assessment and Accreditation of Laboratory Animal Care (AAALAC).

Female C57/B6L mice, 5 mice in each group, were injected with LPS (1 mg/kg) to induce septic shock 30 minutes after oral administration of compounds described herein (formulated in Phosal 50) or the controls dexamethasone (formulated in saline), a corticosteroid, and MCC950 (formulated in 0.5% methylcellulose), an inflammasome inhibitor. The mice were euthanized 2 hours after LPS stimulation, and the whole blood was drawn, processed to plasma, and snap frozen. Cytokine levels in plasma were determined by cytometric bead array assay.

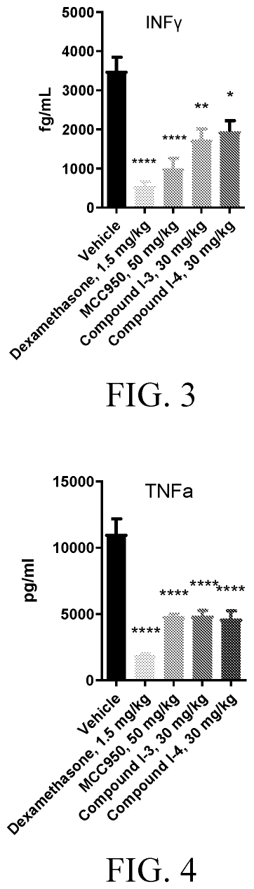

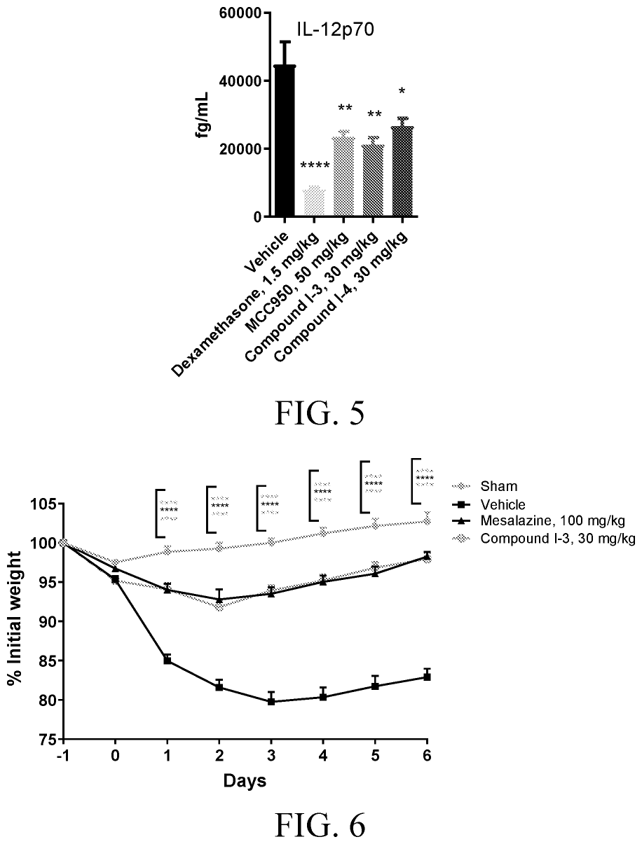

Compound I-3 (30 mg/kg PO, formulated in Phosal 50) and Compound I-4 (30 mg/kg PO, formulated in Phosal 50) caused statistically significant decreases in IFNγ (FIG. 3), TNFα (FIG. 4), and IL-12p70 (FIG. 5) comparable to the positive control inflammasome inhibitor MCC950 (50 mg/kg PO, formulated in 0.5% methylcellulose) when compared to levels seen with vehicle (Phosal 50) treated animals. The positive control dexamethasone (1.5 mg/kg PO, formulated in saline) was also active as expected.

### Example 26: TNBS-Induced Model of Colitis in Mice In Vivo with Dosing from Day-1, “Prophylactic Mode”

All the procedures related to animal handling, care and the treatment in the study were performed according to the guidelines approved by the Institutional Animal Care and Use Committee (IACUC) of WuXi AppTec following the guidance of the Association for Assessment and Accreditation of Laboratory Animal Care (AAALAC).

Female Balb/c mice aged 8 weeks and weighing 18-20 g were housed under standard conditions. The mice were divided into groups with 8-10 mice in each group by body weight. Mice in sham group received intracolonic injection with 50% ethanol (0.1 mL) at day 0, whereas mice in other Trinitrobenzene sulfonic acid (TNBS) colitis model groups received intracolonic injection of 2% TNBS solution in ethanol (0.1 mL) at day 0. The reference compound Mesalazine (Selleck) and test articles described here were dosed from day-1 to day 6. Day 6 was the endpoint, and samples were immediately harvested for the last endpoint. Mesalazine was dissolved in distilled water (pH 7.0) and test articles were dissolved in Phosal 50 PG for delivery by oral gavage.

As noted, dosing of the compounds described herein, as well as the positive control mesalamine, began one day before TNBS administration. Animals in this scenario have not yet developed colitis symptoms before administration of test articles. For this reason, we refer to these conditions as “prophylactic mode”.

Body weight and Disease Activity Index (DAI) scores were recorded daily to assess the colitis. DAI score was the sum of the weight loss score, stool score and bleeding sub-scores. A blinded scoring system was employed to assess the colitis. The DAI scorer blinded to the group information and animal ID was responsible for the stool consistency and bleeding evaluations. If there was no blood visible with naked eyes, the Fecal occult blood (FOB) test was performed with a fecal occult blood test strip (the improved pyramidon method).

Colon tissue morphology, damage and healing were assessed by histopathology. The colons were harvested, mesentery and adipose tissue were carefully removed, and internal contents were removed by rinsing with cold PBS. The colons were Swiss-rolled and fixed in neutralized paraformaldehyde followed by H&E staining. Clinical pathologists reviewed the H&E staining and scored blinded with animal ID. The pathological scoring standards were: crypt architecture (normal, 0-severe crypt distortion with loss of entire crypts, 3), degree of inflammatory cell infiltration (normal, 0-dense inflammatory infiltrate, 3), muscle thickening (base of crypt sits on the muscularis mucosae, 0-marked muscle thickening present, 3), goblet cell depletion (absent, 0-present, 1) and crypt abscess (absent, 0-present, 1).

The plasma was collected at Day-1 for PK and cytokine (IL-1ß and IL-6) analysis by mandibular venipuncture.

The data was compared by ANOVA with post-hoc Dunnett's multiple comparisons test using Graph Pad Prism 7.0 software (San Diego, CA, USA). P-value<0.05 was considered as statistically significant difference. For pairwise comparison of histology scores and cytokines, unpaired t-test was used to calculate statistical significance. Data was expressed as mean±standard error of mean. In figures, statistical significance is denoted by asterisks: *p<0.05 **p<0.01 ***p<0.005, ****p<0.0001.

The study assessed whether Compound I-3 and Compound I-4 could promote the recovery of mice in TNBS induced acute colitis model and compared the data of test groups with that of the mice in Vehicle group.

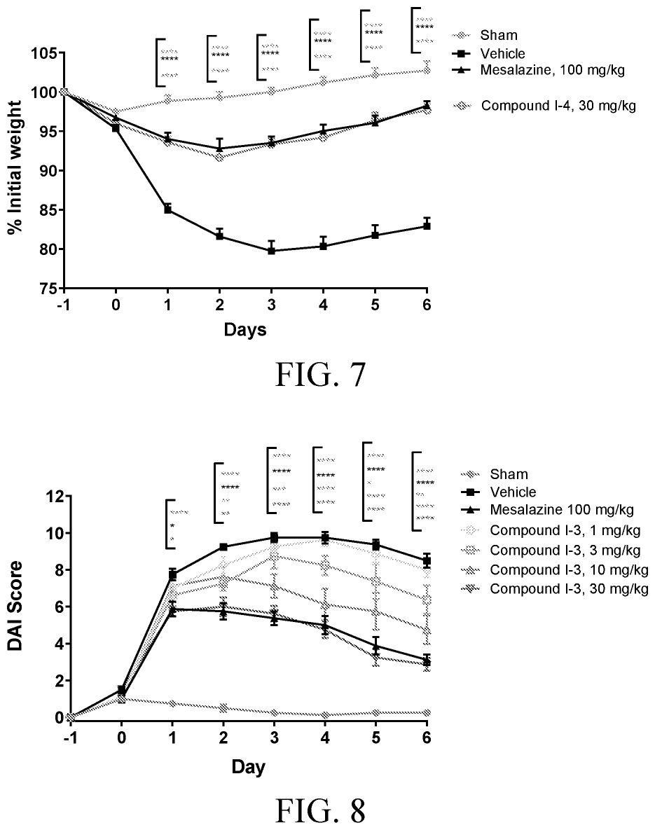

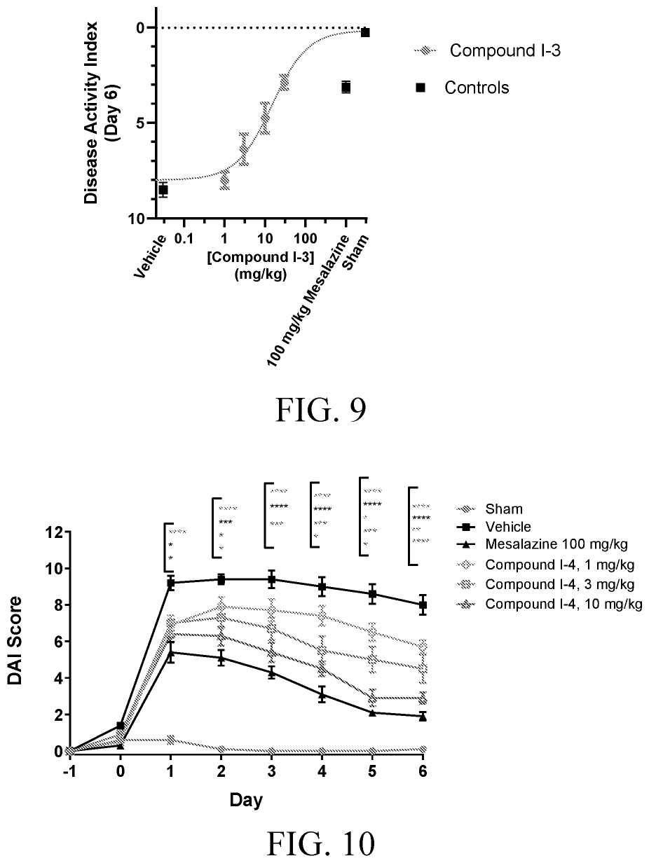

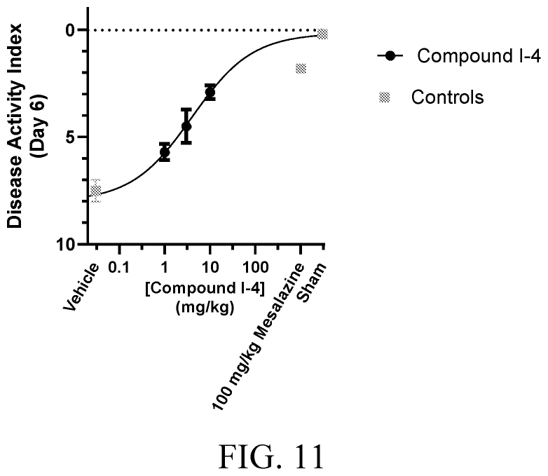

Mice in the Mesalazine (100 mg/kg, QD) group, Compound I-3 (30 mg/kg, PO, QD) (FIG. 6) and Compound I-4 (30 mg/kg, PO, QD) (FIG. 7) groups exhibited a faster recovery rate, showing a markedly less weight loss and more rapid weight gain. Mice treated with Compound I-3 at 1, 3, 10 and 30 mg/kg PO, QD showed a dose-dependent improvement in Disease Activity Index (DAI) scores, less diarrhea, and less bleeding in the late phase of colitis model after TNBS induction (FIG. 8). The dose for half maximal effect or ED50 is 15 mg/kg for Compound I-3 (FIG. 9). Mice treated with Compound I-4 at 1, 3 and 10 mg/kg PO, QD showed a dose-dependent improvement in Disease Activity Index (DAI) scores, less diarrhea, and less bleeding in the late phase of colitis model after TNBS induction (FIG. 10). The dose for half maximal effect or ED50 is 15 mg/kg for Compound I-4 (FIG. 11).

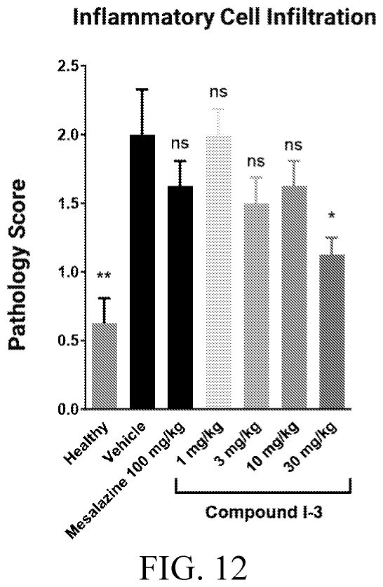

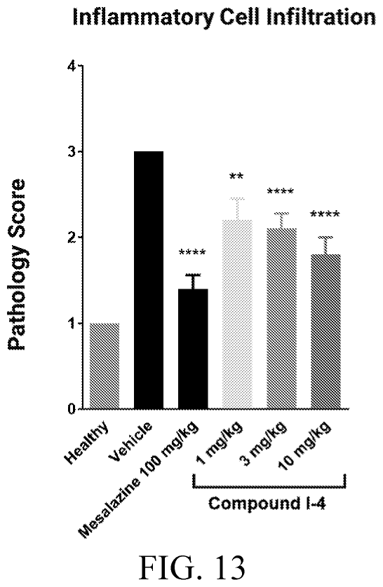

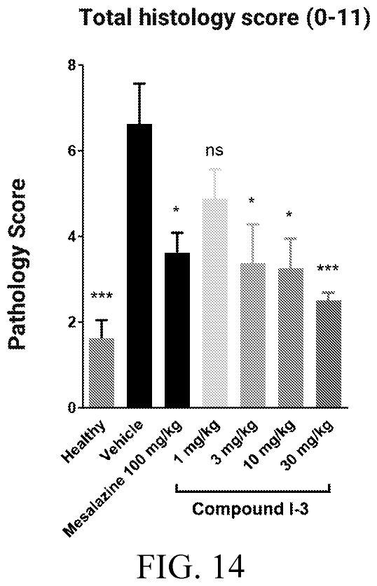

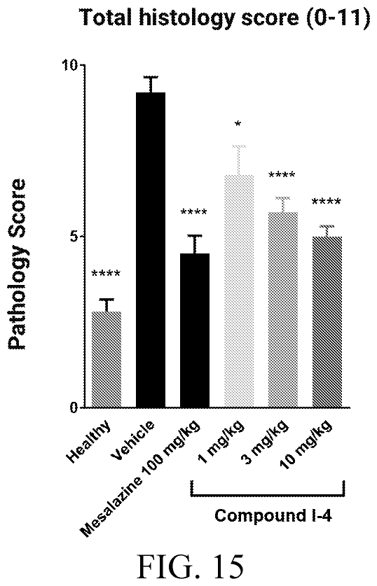

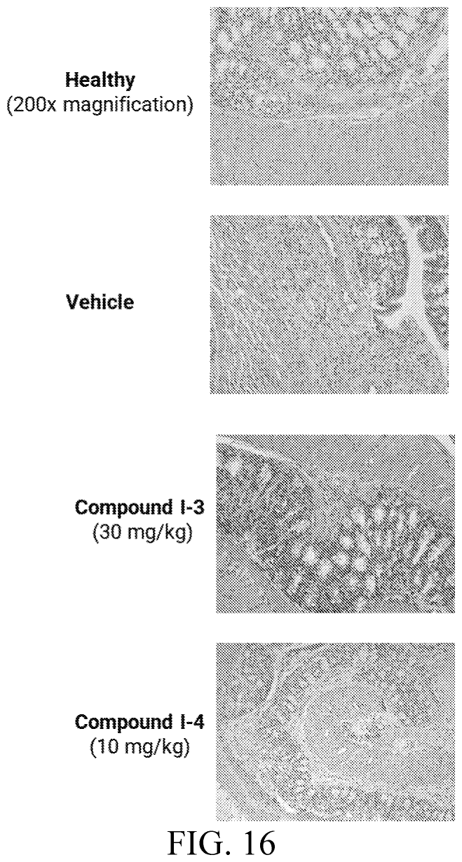

Consistent with the in-life data, mice in the Mesalazine (100 mg/kg PO, QD) group, Compound I-3 (1, 3, 10 and 30 mg/kg PO, QD) group and Compound I-4 (1, 3 and 10 mg/kg PO, QD) group had lower pathological score, which represents less crypt structure failure and less inflammatory cell infiltration (FIGS. 12 and 13) in H&E staining slides, resulting in lower total histology scores given by pathologists (FIGS. 14 and 15). Representative micrographs of H&E-stained colon tissue sections from mice treated with Compound I-3 and Compound I-4 (FIG. 16) show overall tissue architecture, particularly colonic crypt structure, is restored, looking much more like the healthy mice than the vehicle-treated animals, which have large changes in morphology and no visible crypts. The data demonstrate that Compounds I-3 and I-4 promoted the colon recovery and mucosal healing of mice in TNBS-induced colitis model in a dose-dependent manner.

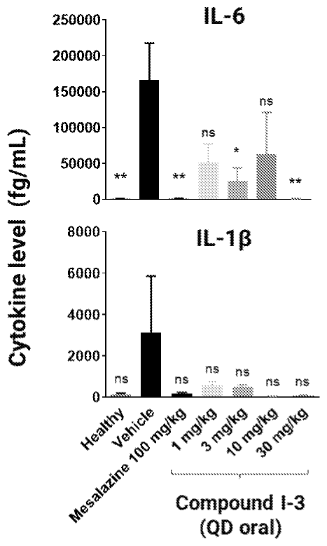

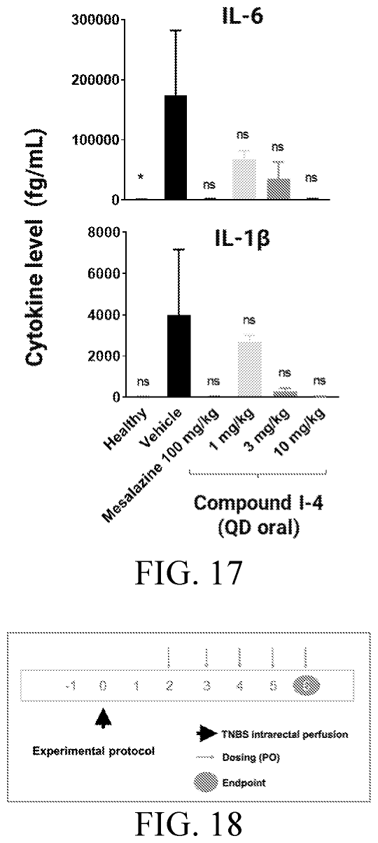

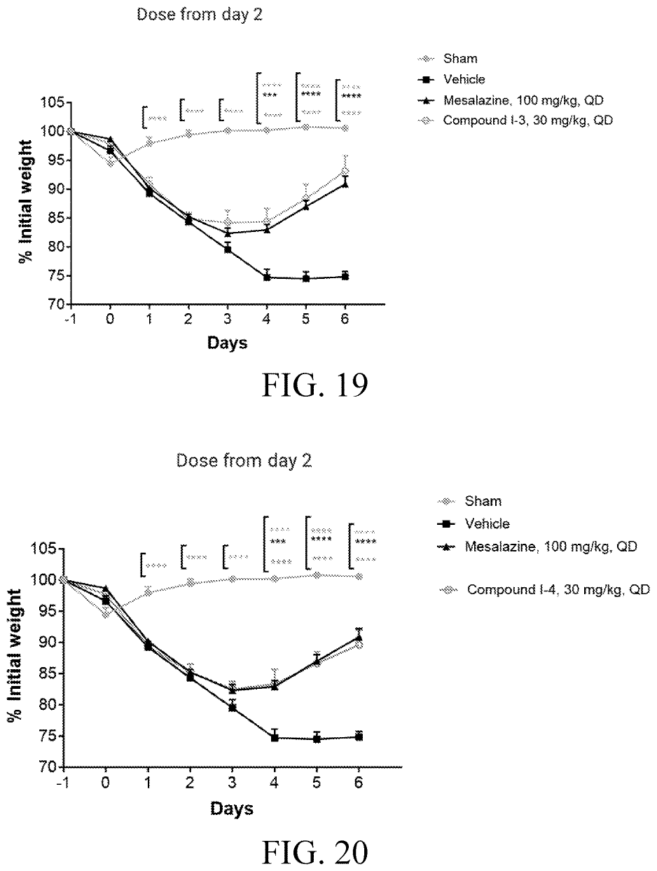

The study further analyzed inflammatory cytokine release in plasma. Mesalazine (100 mg/kg, QD), Compound I-3 (1, 3, 10 and 30 mg/kg PO, QD, FIG. 17) and Compound I-4 (1, 3 and 10 mg/kg PO, QD, FIG. 18) administration decreased IL-1ß and IL-6 in plasma of mice in TNBS-induced colitis model compared with the Vehicle group.

### Example 27: TNBS-Induced Model of Colitis in Mice In Vivo with Dosing from Day 2, “Therapeutic Mode”

Procedures were identical to those in Example 26, except that dosing of the compounds described herein, as well as the positive control mesalamine, began on the second day after TNBS administration, as diagrammed in FIG. 18. Animals in this scenario have developed colitis symptoms before administration of test articles. This is more similar to the clinical scenario with patients with inflammatory bowel disorder presenting with symptoms. For this reason, we refer to these conditions as “therapeutic mode”.

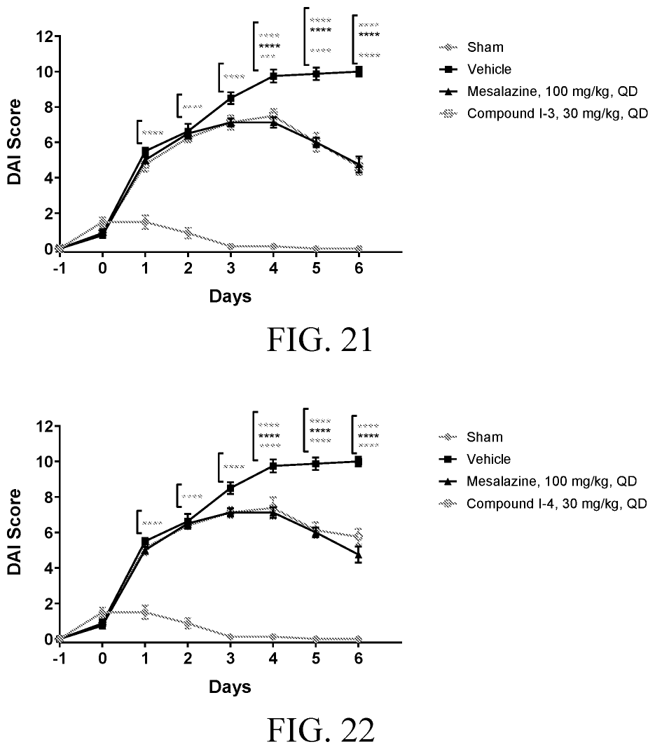

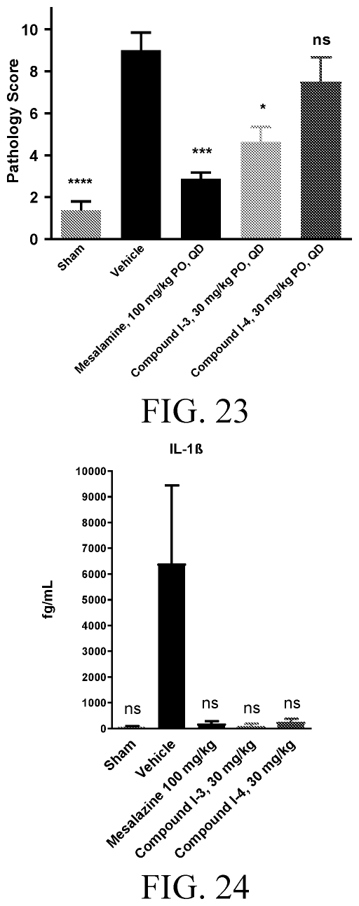

Mice in the Mesalazine (100 mg/kg, QD) group, Compound I-3 (30 mg/kg, PO, QD) (FIG. 20) and Compound I-4 (30 mg/kg, PO, QD) (FIG. 21) groups exhibited a faster recovery rate, showing a markedly less weight loss and rapid weight gain. Mice treated with Compound I-3 at 30 mg/kg PO, QD (FIG. 22) and Compound I-4 at 30 mg/kg PO, QD (FIG. 23) showed improvement in Disease Activity Index (DAI) scores, less diarrhea, and less bleeding in the late phase of colitis model after TNBS induction.

Consistent with the in-life data, mice in the Mesalazine (100 mg/kg PO, QD) group, Compound I-3 (30 mg/kg PO, QD) group and Compound I-4 (30 mg/kg PO, QD) group had lower total histology scores as assessed by pathologist review (FIG. 24). The data demonstrate that Compounds I-3 and I-4 promoted the colon recovery and mucosal healing of mice in TNBS-induced colitis model in “therapeutic mode”.

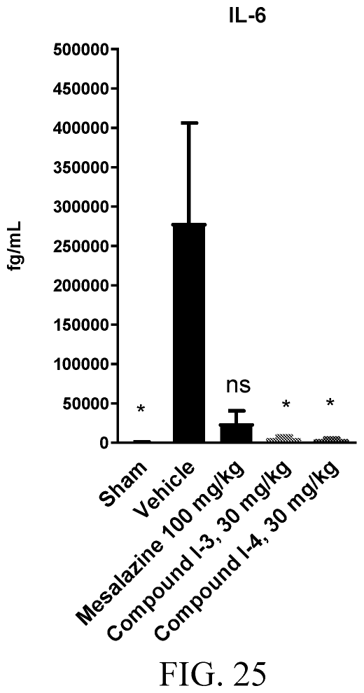

The study further analyzed inflammatory cytokine release in plasma. Mesalazine (100 mg/kg, QD), Compound I-3 (30 mg/kg PO, QD) and Compound I-4 (30 mg/kg PO, QD) administration decreased IL-1B (FIG. 24) and IL-6 (FIG. 25) in plasma of mice in TNBS-induced colitis model compared with the Vehicle group.

### Example 28: Biological Assays

Compounds were evaluated for activity in a TNBS-induced acute colitis model of Crohn's disease in mice. Mice used were 8-week-old (18-20 g) females from the Balb/c inbred strain.

To induce disease, mice in sham group received intracolonic injections of 50% ethanol (0.1 mL) at day 0 while mice in other groups received intracolonic injections of 2% Trinitrobenzene sulfonic acid (TNBS) solution (0.1 mL) at day 0.

The formulation for oral delivery of compounds was prepared as follows. Mesalamine (active agent, positive control) was dissolved in distilled water (pH 7.0), vortexed and sonicated to obtain a suspension. Test articles were dissolved in Phosal 50 PG, vortexed and sonicated to obtain a suspension.

Mice were dosed orally every day, at the concentration and rate indicated in figures. Samples were collected as follows: Blood was drawn 30 min after the first dose on day-1 for pharmacokinetic (PK) analysis. Blood was drawn 30 min after the final dose on day 6 for PK and cytokine analysis. Animals were sacrificed immediately after the blood draw on day 6, with indicated tissue collected for PK analysis.

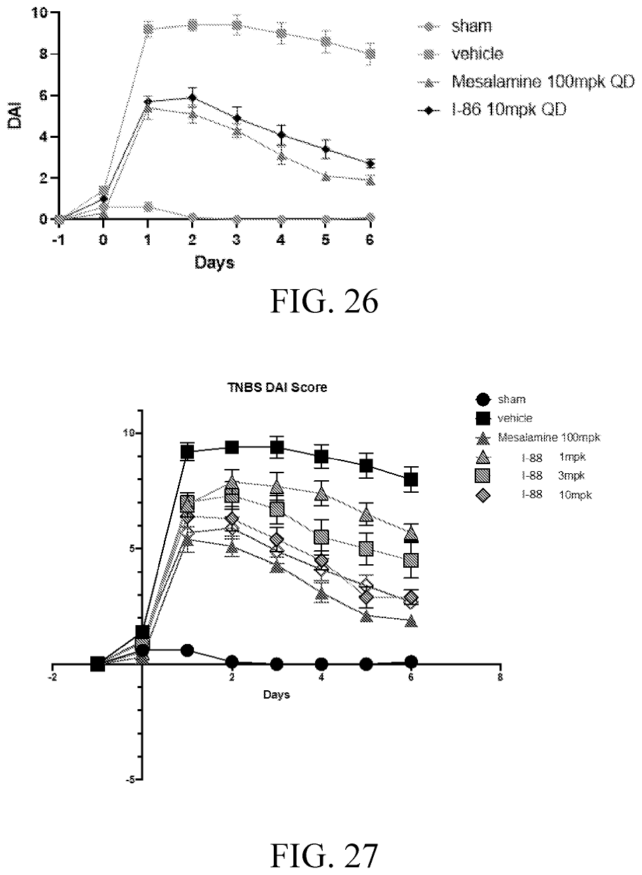

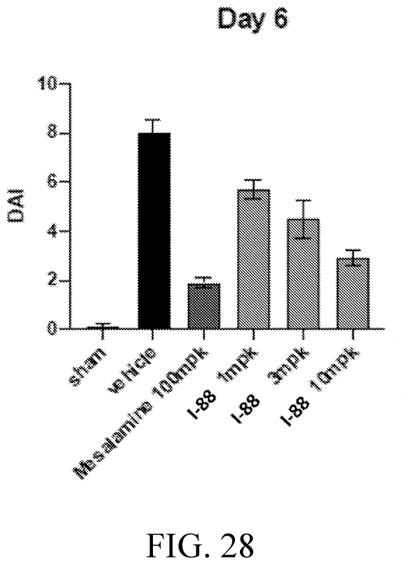

The following observations were made. Disease activity index (DAI) scoring was recorded daily to assess the colitis. See, Table 3 and FIGS. 26-28. Colons from sacrificed animals were fixed and stained with H&E before assessing for pathology. See, Table 4.

TABLE 3
Disease Activity Index (DAI) scoring system (0-12)

| Score | Weight Loss | Stool Consistency | Bleeding |
|---|---|---|---|
| 0 | No loss | Normal | Occult blood positive |
| 1 | 1-5% | Soft yet still formed | Occult blood weak positive |
| 2 | 6-10% | Very soft | Occult blood positive |
| 3 | 11-20 | Diarrhea | Bleeding |
| 4 | ?20% | Watery diarrhea | Bleeding heavily |

DAI score is the sum of the weight loss score, stool score and bleeding sub-scores

TABLE 4
Pathological scoring standards (0-11)

| Items | Score |
|---|---|
| Crypt | 0 - normal |
| architecture | 1 - a few crypts are distorted and a few are lost |
| | 2 - more crypt distortion, more crypt loss |
| | 3 - severe crypt distortion with loss of entire crypts, |
| | more than a third of the total area |
| Inflamma- | 0 - normal |
| tory cell | 1 - only a few inflammatory cells infiltrated in the mucosa |
| infiltration | 2 - multiple areas of inflammatory cells infiltrate the |
| | submucosa |
| | 3 - dense inflammatory infiltrate, more than a third of |
| | the total area |
| Muscle | 0 - base of crypt sits on the muscularis mucosae |
| thickening | 1 - thickening of the crypt is slightly detached from the |
| | mucous muscularis |
| | 2 - thickening of the muscle layer due to moderate |
| | inflammation |
| | 3 - the muscular layer is thickening with severe |
| | inflammation |
| Goblet cell | 0 - absent |
| depletion | 1 - present |
| Crypt | 0 - absent |
| abscess | 1 - present |

### Method 2: Cytokine Production from Human Primary Monocyte-Derived Dendritic Cells

Human primary CD14+ monocytes were isolated from fresh leukopak by CD14 affinity using magnetic beads conjugated to anti-CD14 antibodies and sorted by magnetic-assisted cell sorting (MACS). CD14+ monocytes were cryopreserved in liquid nitrogen until use.

Monocyte-derived dendritic cells were produced by thawing CD14+ monocytes and culturing for 6 days in GM-CSF plus IL-4. Cells were then seeded into 96 well plates for treatment. Test compounds were incubated with cells at 1.0 μM for two or six hours, then cells were stimulated with the TLR agonists LPS or R848 to stimulate with innate immune response. Pro-inflammatory cytokines produced by the cells and present in the supernatant media from the treated cells were quantified using multiplex antibody-based detection.

It is to be understood that while the invention has been described in conjunction with the preferred specific embodiments thereof, that the foregoing description and the examples that follow are intended to illustrate and not limit the scope of the invention. It will be understood by those skilled in the art that various changes may be made and equivalents may be substituted without departing from the scope of the invention, and further that other aspects, advantages and modifications will be apparent to those skilled in the art to which the invention pertains. In addition to the embodiments described herein, the present disclosure contemplates and claims those inventions resulting from the combination of features of the invention cited herein and those of the cited prior art references which complement the features of the present invention. Similarly, it will be appreciated that any described material, feature, or article may be used in combination with any other material, feature, or article, and such combinations are considered within the scope of this invention.

The disclosures of each patent, patent application, and publication cited or described in this document are hereby incorporated herein by reference, each in its entirety, for all purposes.

1. A compound of Formula (IA): 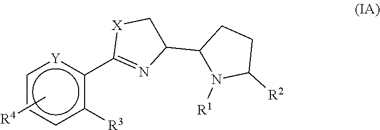
wherein:
R¹ is C₁₋₆alkyl or C₂₋₆alkenyl;
R² is H, C(O)OH, or a masked carboxylic acid;
R³ is H, OH, or OSO₂Z, wherein Z is halogen;
X is O or S;
Y is N or CH;
R⁴ is H or O—(C₁₋₆alkyl-O) n-C₁₋₆alkyl or —(C₁₋₆alkyl-O) n-H; and
n is 1-4;
or a pharmaceutically acceptable salt thereof;
with the proviso that the compound is not (2S,5S)-5-((S)-2-(2-hydroxyphenyl)-4,5-dihydrothiazol-4-yl)-1-methylpyrrolidine-2-carboxylic acid or (2S,5S)-5-((S)-2-(2-hydroxyphenyl)-4,5-dihydrooxazol-4-yl)-1-methylpyrrolidine-2-carboxylic acid.

2. The compound of claim 1, wherein R¹ is C₁₋₆alkyl.

3. The compound of claim 1, wherein R¹ is C₂₋₆alkenyl.

4. The compound of claim 1, wherein R² is C(O)OH.

5. The compound of claim 1, wherein R² is a masked carboxylic acid.

6. The compound of claim 1, wherein R² is a biotinylated masked carboxylic acid.

7. (canceled)

8. The compound of claim 1, wherein R², R³ and/or R⁴ is H.

9. (canceled)

10. The compound of claim 1, wherein R³ is OH.

11. The compound of claim 1, wherein R³ is OSO₂F.

12. The compound of claim 1, wherein X is O.

13. The compound of claim 1, wherein X is S.

14. The compound of claim 1, wherein Y is N.

15. The compound of claim 1, wherein Y is CH.

16. (canceled)

17. The compound of claim 1, wherein R⁴ is —O—(CH₂CH₂) n-C₁₋₆alkyl, such as —O—(CH₂CH₂)—O—(CH₂CH₂)—O—CH₃.

18. (canceled)

19. The compound of claim 1, that is of formula (II), (III), (IV), (V), (VI), (VII), or (I):

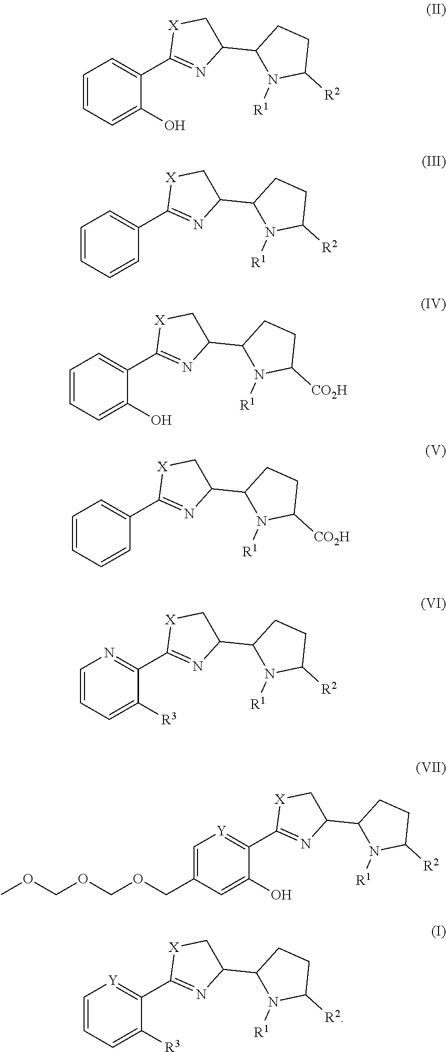

20-25. (canceled)

26. The compound of claim 1, that is Compound A, Compound I-1, Compound I-2, Compound B, Compound I-3, Compound I-4, or Compound 12: 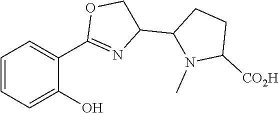 [SMILES: CN1C(C(=O)O)CCC1C1COC(c2ccccc2O)=N1]
Compound A (5-(2-(2-hydroxyphenyl)-4,5-dihydrooxazol-4-yl)-1-methylpyrrolidine-2-carboxylic acid); 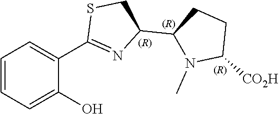
Compound I-1 ((2R,5R)-5-((R)-2-(2-hydroxyphenyl)-4,5-dihydrooxazol-4-yl)-1-methylpyrrolidine-2-carboxylic acid); 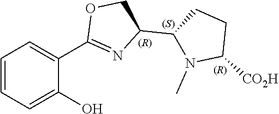
Compound I-2 ((2R,5S)-5-((R)-2-(2-hydroxyphenyl)-4,5-dihydrooxazol-4-yl)-1-methylpyrrolidine-2-carboxylic acid); 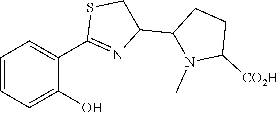 [SMILES: CN1C(C(=O)O)CCC1C1CSC(c2ccccc2O)=N1]
Compound B (5-(2-(2-hydroxyphenyl)-4,5-dihydrothiazol-4-yl)-1-methylpyrrolidine-2-carboxylic acid); 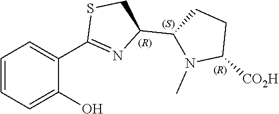
Compound I-4 ((2R,5S)-5-((R)-2-(2-hydroxyphenyl)-4,5-dihydrothiazol-4-yl)-1-methylpyrrolidine-2-carboxylic acid); 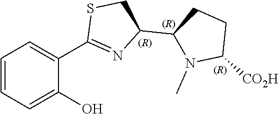
Compound I-3 ((2R,5R)-5-((R)-2-(2-hydroxyphenyl)-4,5-dihydrothiazol-4-yl)-1-methylpyrrolidine-2-carboxylic acid); 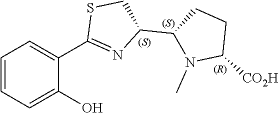
Compound I-12 ((2R,5S)-5-((S)-2-(2-hydroxyphenyl)-4,5-dihydrothiazol-4-yl)-1-methylpyrrolidine-2-carboxylic acid).

27. (canceled)

28. The compound of claim 1, that is Compound C, Compound D, Compound E, Compound F, Compound G, Compound H, Compound J, Compound K, Compound L, Compound I-5, Compound I-6, Compound I-7, Compound I-8, Compound I-9, Compound I-10, Compound I-11, Compound I-13, Compound I-14, Compound I-15, Compound I-16, Compound I-17, or Compound I-18:

| Compound | |
|---|---|
| # | Structure |
| C | 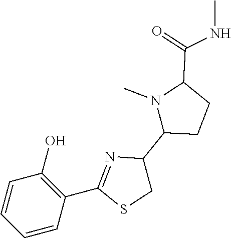 [SMILES: CNC(=O)C1CCC(C2CSC(c3ccccc3O)=N2)N1C] |
| D | 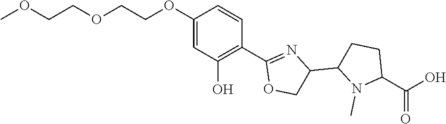 [SMILES: COCCOCCOc1ccc(C2=NC(C3CCC(C(=O)O)N3C)CO2)c(O)c1] |
| E | 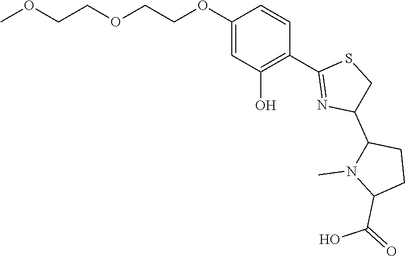 [SMILES: COCCOCCOc1ccc(C2=NC(C3CCC(C(=O)O)N3C)CS2)c(O)c1] |
| F | 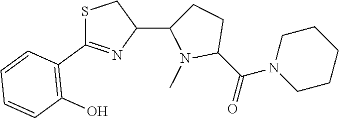 [SMILES: CN1C(C(=O)N2CCCCC2)CCC1C1CSC(c2ccccc2O)=N1] |
| G | 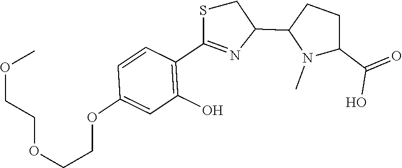 [SMILES: COCCOCCOc1ccc(C2=NC(C3CCC(C(=O)O)N3C)CS2)c(O)c1] |
| H | 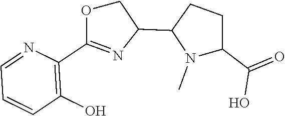 [SMILES: CN1C(C(=O)O)CCC1C1COC(c2ncccc2O)=N1] |
| J | 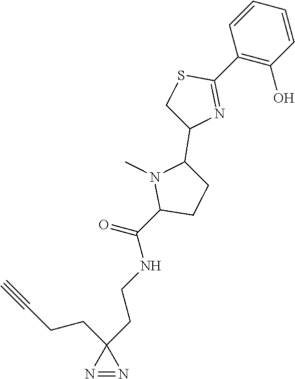 [SMILES: C#CCCC1(CCNC(=O)C2CCC(C3CSC(c4ccccc4O)=N3)N2C)N=N1] |
| K | 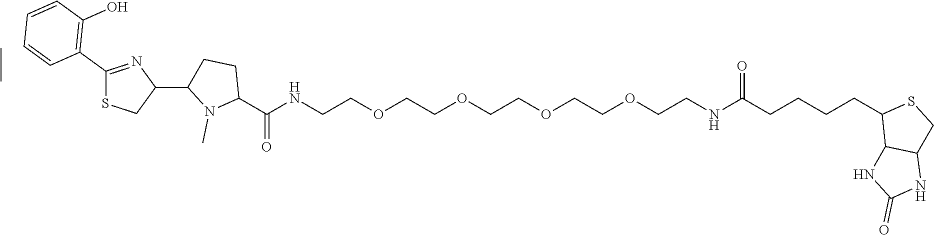 |
| L | 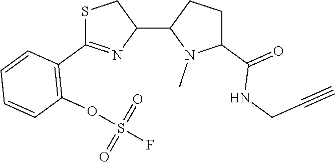 [SMILES: C#CCNC(=O)C1CCC(C2CSC(c3ccccc3OS(=O)(=O)F)=N2)N1C] |
| I-5 | 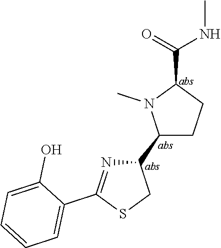 [SMILES: CNC(=O)[C@H]1CC[C@@H]([C@@H]2CSC(c3ccccc3O)=N2)N1C] |
| I-6 | 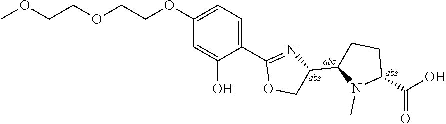 [SMILES: COCCOCCOc1ccc(C2=N[C@H]([C@H]3CC[C@H](C(=O)O)N3C)CO2)c(O)c1] |
| I-7 | 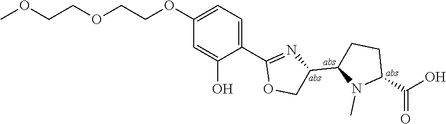 [SMILES: COCCOCCOc1ccc(C2=N[C@H]([C@H]3CC[C@H](C(=O)O)N3C)CO2)c(O)c1] |
| I-8 | 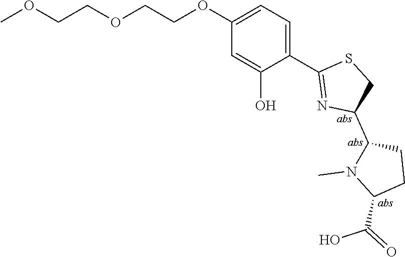 [SMILES: COCCOCCOc1ccc(C2=N[C@H]([C@@H]3CC[C@H](C(=O)O)N3C)CS2)c(O)c1] |
| I-9 | 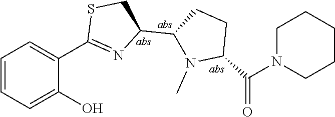 [SMILES: CN1[C@@H](C(=O)N2CCCCC2)CC[C@H]1[C@@H]1CSC(c2ccccc2O)=N1] |
| I-10 | 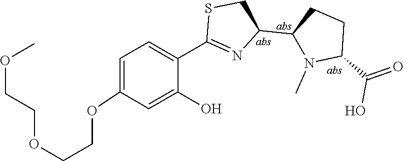 [SMILES: COCCOCCOc1ccc(C2=N[C@H]([C@H]3CC[C@H](C(=O)O)N3C)CS2)c(O)c1] |
| I-11 | 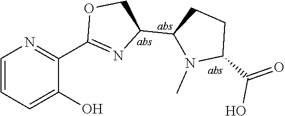 [SMILES: CN1[C@@H](C(=O)O)CC[C@@H]1[C@@H]1COC(c2ncccc2O)=N1] |
| I-13 | 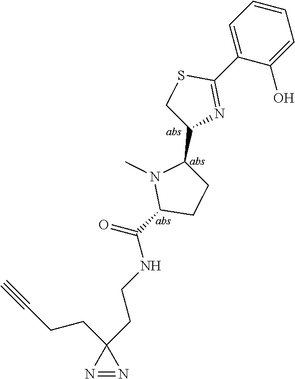 [SMILES: C#CCCC1(CCNC(=O)[C@H]2CC[C@H]([C@@H]3CSC(c4ccccc4O)=N3)N2C)N=N1] |
| I-14 | 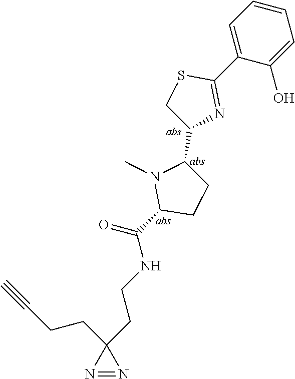 [SMILES: C#CCCC1(CCNC(=O)[C@H]2CC[C@@H]([C@@H]3CSC(c4ccccc4O)=N3)N2C)N=N1] |
| I-15 | 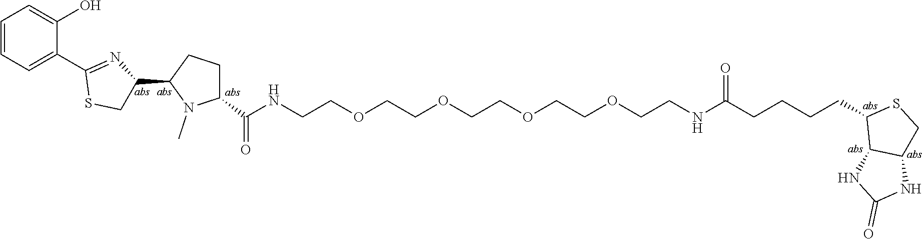 [SMILES: CN1[C@@H](C(=O)NCCOCCOCCOCCOCCNC(=O)CCCC[C@@H]2SC[C@@H]3NC(=O)N[C@@H]32)CC[C@@H]1[C@@H]1CSC(c2ccccc2O)=N1] |
| I-16 | 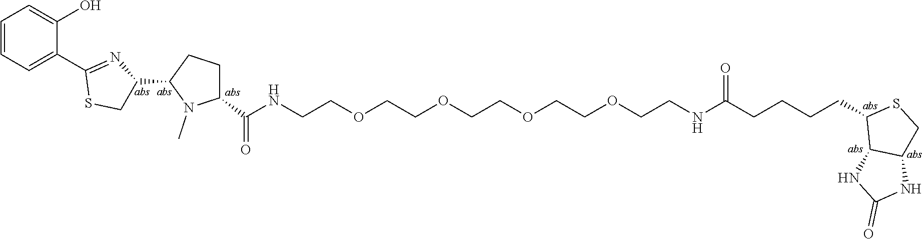 [SMILES: CN1[C@@H](C(=O)NCCOCCOCCOCCOCCNC(=O)CCCC[C@@H]2SC[C@@H]3NC(=O)N[C@@H]32)CC[C@H]1[C@@H]1CSC(c2ccccc2O)=N1] |
| I-17 | 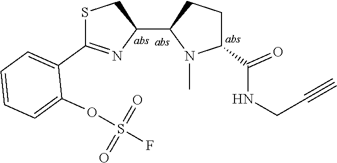 [SMILES: C#CCNC(=O)[C@H]1CC[C@H]([C@@H]2CSC(c3ccccc3OS(=O)(=O)F)=N2)N1C] |
| I-18 | 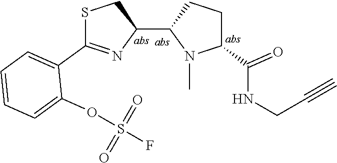 [SMILES: C#CCNC(=O)[C@H]1CC[C@@H]([C@@H]2CSC(c3ccccc3OS(=O)(=O)F)=N2)N1C] |

29. (canceled)

30. A pharmaceutical composition comprising a pharmaceutically acceptable excipient and (i) a compound of claim 1, (ii) (2S,5S)-5-((S)-2-(2-hydroxyphenyl)-4,5-dihydrothiazol-4-yl)-1-methylpyrrolidine-2-carboxylic acid, or (iii) (2S,5S)-5-((S)-2-(2-hydroxyphenyl)-4,5-dihydrooxazol-4-yl)-1-methylpyrrolidine-2-carboxylic acid.

31. A dosage form comprising the compound of claim 1.

32-34. (canceled)

35. A method of (i) treating inflammation, (ii) decreasing inflammation, (iii) decreasing an inflammatory marker, (iv) treating inflammatory bowel disease, or (v) treating sepsis in a subject in need thereof, comprising administering the compound of claim 1 to the subject in need thereof.

36-40. (canceled)

41. A probe comprising one or more compounds of claim 1.
